SEQUENCE LISTING (303 sequences)

SEQ ID NO: 1 - DNA, 2368 bp, Homo sapiens
gactgttctcgcgttcgcggacggctgtggtgttttggcgcatgggcggagccgtagttacggtcgactggggcgtcgtccctagcccgggagccgggtctctggagtcgcggcccggggttcacgatgtccgacgaggaagcgaggcagagcggaggctcctcgcaggccggcgtcgtgactgtcagcgacgtccaggagctgatgcggcgcaaggaggagatagaagcgcagatcaaggccaactatgacgtgctggaaagccaaaaaggcattgggatgaacgagccgctggtggactgtgagggctacccccggtcagacgtggacctgtaccaagtccgcaccgccaggcacaacatcatatgcctgcagaatgatcacaaggcagtgatgaagcaggtggaggaggccctgcaccagctgcacgctcgcgacaaggagaagcaggcccgggacatggctgaggcccacaaagaggccatgagccgcaaactgggtcagagtgagagccagggccctccacgggccttcgccaaagtgaacagcatcagccccggctccccagccagcatcgcgggtctgcaagtggatgatgagattgtggagttcggctctgtgaacacccagaacttccagtcactgcataacattggcagtgtggtgcagcacagtgaggggaagcccctgaatgtgacagtgatccgcaggggggaaaaacaccagcttagacttgttccaacacgctgggcaggaaaaggactgctgggctgcaacattattcctctgcaaagatgattgtccctggggaacagtaacaggaaagcatcttcccttgccctggacttgggtctagggatttccaacttgtcttctctccctgaagcataaggatctggaagaggcttgtaacctgaacttctgtgtggtggcagtactgtggcccaccagtgtaatctccctggattaaggcattcttaaaaacttaggcttggcctctttcacaaattaggccacggccctaaataggaattccctggattgtgggcaagtgggcggaagttattctggcaggtactggtgtgattattattattatttttaataaagagttttacagtgctgatatgaccctgttgtcaccccagctgaatttcttatgaccctcccaaaccaaagctcagatggggtcagaagagcttcatagaaagttgggcaaaacaggctagcaattgcaaagtcaggctttgaccaacatatttctttgcactgaggccttgctgctgtggatacggaaatggttaagtactgtgcttcctcagcagctgggctgtcagggccatagtagctccctttggagaacagggaaagcctggaggcttcccaggtggcccagcgtggtgtcctgtcagcttcctctttaggaacccaccagagggcagcaagctcctttcacttcgctagtaagaacccctccgtttttgtgtgtttttgtttttgttttctggagacaaggtcttgctttgtcacccaggctggagtgcagtgtcgtgatcaaggttcactgaagccttgacgctgtgggcactgcctcagccgcccaagtatctgggaccacaggcgtgcaccaccatgcatagctaatttattttttgtagagacagggtctccctgtgttgaccaggttggtctcgaactcctgggctcaagcagtcctcctgccttggcctcctaaagtgctgggatcacaggcgtgagccactgcgcccagcccactgctagtttgactttttataattgaacctcctggctatgccctgagatcagcgctattttgtaaaccgctgaggtatggataggaacgagtagatcagacctcttgaaaatgcttattcttcctcccttttattttttgtctcttttaagatggtaaaatggttctcagggattcctgccaatactttgaattattttttcctctccatggtatcagtgttcatttccccagttcttgcacaccgctttctgttttggcagttctgccaggcaagccctgtgttccttgggactggttttgctgtggttggatacagataccagcttgccttgatgggattggtattgctgtgtgcttccagccacaggttctcacactcaattccaaagccttcctattgggcgaattccctcaaactctatttgacctgacagccatacgtattcccctctggtagccacagacatgctgtgtttaccaatgtttgctgtttaaattgcatgttctaattccacgtattttccagtctcttttataaagtctcagactataataaacacagcttgcccagtttaaaaaaaaaaaaaaaaa

SEQ ID NO: 2 - protein, 223 aa, Homo sapiens
MSDEEARQSGGSSQAGVVTVSDVQELMRRKEEIEAQIKANYDVLESQKGIGMNEPLVDCEGYPRSDVDLYQVRTARHNIICLQNDHKAVMKQVEEALHQLHARDKEKQARDMAEAHKEAMSRKLGQSESQGPPRAFAKVNSISPGSPASIAGLQVDDEIVEFGSVNTQNFQSLHNIGSVVQHSEGKPLNVTVIRRGEKHQLRLVPTRWAGKGLLGCNIIPLQR

SEQ ID NO: 3 - DNA, 2610 bp, Mus musculus
atctgtttccgtacttgcgcgttggctgagctgttttggccccggggaggacccgtagcctcagcctcggctggcgtccgtagccgggaaacccagaactctagcttgaggtccgcgatgtcgggtgaagacgttccgcaccgggcagagtcctccgaggcccgtgcggccgcggtcagcgacatccaggatctgatgcgacgcaaggaggagatcgaggcggagatcaaggctaattacgacgtcctggagagccaaaaaggaattggcatgaacgaaccgctggtggactgtgagggctatccccgggcggatgtggacttgtaccaggtccgaacagcaaggcacaacatcatctgtctccagaatgatcacaaggcactgatgaagcaagtggaggaggccctgcaccagctgcacgctcgggacaaagagaagcaggctcgggacatggctgaagcccgagaagaggccatgaaccgcaggctggcctccaacagccccgtcctgccccaggcctttgccagagtgaacagtatcagccccggttccccagccagtattgcgggcctgcaagtggatgacgaaattgtggagttcggctccgtgaacacccaaaacttccagtcagtgcagaacgtgggcactgtggtgcagcatagcgaggggaagcccctgaatgtgacggtgatccgcagaggagagaagcaccagctcagactgattccaacccgctgggcaggaaaaggactgctgggctgcaacattattcctctccagagatgactgtttcctgggatctgcctgcaggaagctgcctcagctggccccgtgcttgggcctggaggcgtttcctcgttctctaggctcccttaagtgtaaggatctggagaagaatggtcgaagcctgggcatcgaggtggaagagacgctttggctgcctgatgtaatctctctgggttgaggcattattaaaagtgtgatttgtgcctagctacgtcttgtgcaaattaggccatagcctgtctgggaattctctagattatgagccagtgagtggggatgactctggcgagtaatgatttgatgtcattttccttttggagacggactcagtgtgtaatcctgactatcctggaattccctgtgtagaccaggctggccttgagctgacagagatccccctgcctctgcctcattagtattgggatgagaggcatatactgacatggctggctagactctgaggaatatgtattatacatatataatttatacttatttttgagaatttcgtacatgcacatagtacatttctatatttcctgtcatctcaagtccctctcagacctcccctcgtacccacttcccaggttcttgccctcttttgttttttttctttttaaataaataacctactaagtgccatttgtgctgtcagtatattcatggtgtgggatcttccactggaggaatgtggtcaaccttccagagaccacacccctaacctccacaggacttgagtacccctactttttcttggaagcagggtctcatgtagcccagtccagcctcagatttgccttgaagctgaagatgttctcttctgctgtctctccctccaatgtccaactgcagtatcacccaccgcctaggggtgggggcttgctggagaccggatctcacaagttcagactggcctggagctcagtgtacccgcactggccttacccttgggatcttgctgccctggtctcctggtattatagtgtatgccaccaaaccttgttacaggttagagaaactgtcagataaagccagtcttgtagctgtgtgattgtgaccactgtcagtcccaccaggatgccctgtgagctcacagccaagcccaggaggagttgggaaggaagtcctgcgaagcaaacgagttcctgggccgcagtccggttgattcttgggtgggaaactgaaagccggagctcagactctaggcaagtgtccctccgtgggctgagcagctggcagtctctaccctggagcagtgctttgagagcctgatgcatcctctgaggctgaggctgtggagtcaccgtagccccattaagaaatcagcaaagaccaggtgtggtggctcaaacgttttaatcccagcactcaggaggcaggggcaggcggatttctgagttaaggccagcctggtctacagagtgagctccaggacagccaaggaaggctacacagaaaaattctgtctcaaaacaaaatttgcaaaggtcacaggtcacgaggccttgcaggttacccagtatgatattggcttcctcttcagtaacccactggaaaccagcaagcttcctttacttctacagtaaaagaccccgcagatgggcctgatttggcttttatagttgagcctccccaccgggcggtggtggtgtacacctttaatcccagcactcaggaggcagagagaggcaggcagatctcagagtttgaggctagcctggtctacagagtgagttccaggacagccagggctacacagagaaaacctgtatggaaaacaaaaataaaacaaatttaaaaaacaaaaaaaaaaaaaaaaaaaaa

SEQ ID NO: 4 - protein, 222 aa, Mus musculus
MSGEDVPHRAESSEARAAAVSDIQDLMRRKEEIEAEIKANYDVLESQKGIGMNEPLVDCEGYPRADVDLYQVRTARHNIICLQNDHKALMKQVEEALHQLHARDKEKQARDMAEAREEAMNRRLASNSPVLPQAFARVNSISPGSPASIAGLQVDDEIVEFGSVNTQNFQSVQNVGTVVQHSEGKPLNVTVIRRGEKHQLRLIPTRWAGKGLLGCNIIPLQR

SEQ ID NO: 5 - DNA, 669 bp, Mus musculus
atgtcgggtgaagacgttccgcaccgggcagagtcctccgaggcccgtgcggccgcggtcagcgacatccaggatctgatgcgacgcaaggaggagatcgaggcggagatcaaggctaattacgacgtcctggagagccaaaaaggaattggcatgaacgaaccgctggtggactgtgagggctatccccgggcggatgtggacttgtaccaggtccgaacagcaaggcacaacatcatctgtctccagaatgatcacaaggcactgatgaagcaagtggaggaggccctgcaccagctgcacgctcgggacaaagagaagcaggctcgggacatggctgaagcccgagaagaggccatgaaccgcaggctggcctccaacagccccgtcctgccccaggcctttgccagagtgaacagtatcagccccggttccccagccagtattgcgggcctgcaagtggatgacgaaattgtggagttcggctccgtgaacacccaaaacttccagtcagtgcagaacgtgggcactgtggtgcagcatagcgaggggaagcccctgaatgtgacggtgatccgcagaggagagaagcaccagctcagactgattccaacccgctgggcaggaaaaggactgctgggctgcaacattattcctctccagagatga

SEQ ID NO: 6 - DNA, 16 bp, Artificial Sequence
  Antisense oligonucleotide
ctctatgggtgccagc

SEQ ID NO: 7 - DNA, 16 bp, Artificial Sequence
  Antisense oligonucleotide
ctctatctgagcacac

SEQ ID NO: 8 - DNA, 16 bp, Artificial Sequence
  Antisense oligonucleotide
gtatttttagccagac

SEQ ID NO: 9 - DNA, 16 bp, Artificial Sequence
  Antisense oligonucleotide
ggccaatacgccgtca

SEQ ID NO: 10 - DNA, 2610 bp, Mus musculus; identical to SEQ ID NO: 3

SEQ ID NO: 11 - DNA, 28000 bp, Mus musculus
gtgtagcccctggctgtcctggaactcactctgtagaccaggctggccttgaactcagggatcagcctgcctctgtgtccctagtgctgggagtaaaggcatgtgccaccatggtcctcttaaatggttttcttagctgtcaaatatgggcaccaatacatatggtttctttgggttgttgaactatcacatataggcatgtagaactgtatttgagattctctgtctcattcaggttttacattatatcttcatgtgcctcaccgctgagtaaaactctcattccttattactattttttttaaaaacatgcttttttaaaaaacaaaataaagagaaaccctgtcttgaaaaatcaaaaccaaccaaccaaccaaccagaaaaacccaaagaacttactttatacttcctgtgcatcagtgttttgcctgagtgtgtgtctgtgtgacggtgtcagagggtggagttgcagacagttgtgagccgccatgcagatgttgagagttgaacctgggaagagcagtcagtgcccttaactgctgagccatctctccagcctccttattactatttaaaaatttttatttttatcatatgtatgtgagttggctagttttatgtcaacttgatgcaacctagagttgtctaaaggaagatggaaactcaattgaaaaaatgcctccttaagttccacctgtagggcattgccttaattagttattgatgggagtgtgttctacccattttgggctggtggtcctgagttctataagaaaataggctgagtaagacacaaggagcaagtcagtaagcagcacacctccattggctcctgcctccaggctcctgcactgtttgaattcttgtcctgatttccttaaatgatgaacagttatataaggaaacgtaagccagatgaagcttttccttcctacattgctttggcctttgtactggctggttttgtgtcaccttgacccgggctggatttatcaccgagaaaggagcttcagctggagaagtgccgccatgagatccagctgtaaggcattttctcaattagtgatcaaggggaaaggccccttgtgggtggtgccagctctgggctggtagtcttagttctataagaaagcaagctgagcaagccaggggaagcaagccagtaaagaacatccctgcatggcctctgcatcagctcctgcttcctgacctgcttgaggtccagtcctgactttggtgatgaacattgttgtggaagtgtaagctaaataaaccctttcctccccactttgcttgttggtcatgatgtttgcgcaggaatagaaaccttgactaacacagccatgctattttatcacagcaatagaaccttaactaagacagtgtgcaagtgttttgcctgcatatacgtatgtgtcctaagtgcgtgctggatggttgtggaggtcaggtgagggcattggagaccctggagctagagtaacagacagttgtaagcttccatgtaagtactgggaacccaggccctctgcaagagctttaagtatgttcttaacctctgaaccatctctctaccccacaccccagtttctatgtttacctacatttatagaggcctgacacagtttcatattctttaccacttcagtcttgtggggaattcatgagaggagactgctcagatagagttttaatctgatagtctttattagtgagccagtacaacactaagtgtttgggaccccagtggagcactgagcctttttcagggtaagctttttgagcacaaaaaccgggtcctggggtgatataccttagttcacaagagcaattagccaggagcacaaccactgaagccaaaaagcaaggttcgtgcatggagagactttcccacagctgtggacttggatggcttccggctttgttttagttttggcaggtggtgctgtctgtgtgctgtgttttccagcctgagtggtactttctttatggagtcggttgtactaaggtctgggggcctgttacaccagcacctcgtgacagaggtgaaagtcaataaatacttggacacccttaagagacacgttgctagggtgtgtgtactctgatcaggatccaaagggaataaattagtaaataaaaatgaaaaaaaagagcgagagatattactgttagtgtcacacaataaggtcagagagcagaaggtgaaattatttaaggtcacatggaatatatatagtcggtgttaatggtccaactttgaacataggttgtaccatgaggatccccagcccgaaagtggctggggatatagctgagttggtagagtgttcctgatccccaatatttcataaaccatacctggtggtacatgcctgtaatccctgcctgtggagggagcagatggagtatcagatgctcaaaggcactgggcacactaggttataaagaaggaaaaaaaaaaaggtcttgaagttttgatgaggataggttgagggaggtgcgaaatggatagaatgggagtggggggttagagggtgtctgtgatcatgacaataagaaatagaatattctaaaaacttggggagagcttcaaggtcgccttcatctacacaatgagttagaggcctgcctgagatgcagtaaatcttgtctggaaaaagaaaaagaaaagaaacagaaaacaaaaaacaaatcgcttgaaagcctctttcaaacactcaggggcttcgtagatgttaagaactgaagtgtcaaggttggcaagactggatcctcttacatttatacagctggtatccagtttactcagccttcagatgagtgaatgaaaaaaagtgtaagccagatgtggtggctttaatcccagcacttgggagcagaggcaggaggatttctgagtttgaggccagcctggtctacagagtgagttccaggacagccagggatacacagagaaaccctgtcttgaaaaacggggtggggtgggggggtggggaaacggacggatggagggacggggacagaatgaaaaagaaaggaatgaacaagaaaagtggaaatgagtcaggataaagtgggagtaaatacctatgtcctctatgggcctatttatctctaccgggtcatctgaaaaccacgtggtctatctagccggtgaccccgcccacttccaagctccacccttcatctgtttccgtacttgcgcgttggctgagctgttttggccccggggaggacccgtagcctcagcctcggctggcgtccgtagccgggaaacccagaactctagcttgaggtccgcgatgtcgggtgaagacgttccgcaccgggcagagtcctccgaggcccgtgcggccgcggtcagcgacatccaggatctgatgcgacgcaaggaggagatcgaggcggagatcaaggctaattacgacgtcctggagagcgtgagtgtggcggggaggcctgcggacgaggctcggggtcgctgccaagcagtgtcctagagggggtctttgctgcgtgccacacgcccctttaagcattttttctttacgtgggattaattttcattttcggaagcattaactttgcattttaaagttttttgtttgtttgtttttaacgcagcccctaagctggcttcgaactcgtgatcctcctgcttcatctttctgagtgcgggaatcgcgggcctgtgcaaccatgatataggccttagtgaaactgcctcggtcgcaaggatagactatttgctgaggcatagtgataatcgctactgtcactttctgtaggtgacattaccaggtttggcctcgccacccgcttttctttaggactttataaatgtcaactgggagaattaattgggcatcttgttaaaatgcacgttgtagccaggggaggcatccctatttaagcccagcgtttagagagtggaggcagggggatcaggacttgagggttattcttgcctacatagtaagctcgaggctgtcctgggttatattagaccttgtctcaaaaaaaaaccaaaaaaaaaaaaaaaaaaaaaaaacaaaaaagaaaagtaaaaatgaaatgcagactctaattgggatagggaaaagactctggattttaagaaaaccctcacctctgctaattgctcttttaagtggagccagattaagcacattgtctttactgtaaaaggggaaacagggccagagtggagccaatgcttgcccagttaggaagggggaatggttggctctgtgatctgtttacctcctaagcagatatatttttcataaaaccccaagtcagtgtatagaggaaggggaggctaaatcacactcattgctgggcacaatgacatctgggtgatgggtgccagatgacggtgtcatatagaggttctacaggccaggtaggtgctgaccatgtgcctgaaaggtactatcttgtatgtgtgctgtgccaggaaactgtcacagcagtattgttaacttgtgtccctgaagctcaaaggggcggagtcaggggctggagagatggctcagtggtttaaaggcactggccatcataaggactggagttgagatccagaatttactactgtgtaagttcacacgcccgtggagacgtttaaacagacacaaatctctttaaagaaaaaaaaacacaaaaacccgtgtgtgtgtgtgtgtgtgtgtgtgtgtgtacacatatatctgtgtatgtatggatacacatatatgtgtatatatgtatgtgtatatgtatgtatgtatatgtgtgtgtgtatacacatatatctgtgtgtgtgtatgaaaggagaagtcacttgtttaaagtcacaggcctccggcttggtaaagcttaaaacagataaaaagcaattttttgttttgttttgtcttgtttttcgaatgagagtttctttttgtagctctggctgtcctgaactcagagatccaccagcctctgcctctcaagtgctaggatttaaggcgtgcactaccatcggctggcaaaaagcaatttcttaaactgcagtaacttgcctaactccagtagaatcagacatctcacgtattgtcttttggtattggcctaccctttactatttagctgggtgtggtgttgcatacctgcagcccgaagcaagcaaaagcatggagattgccacacgttcagggccagtctgttttacacagtgagctctaggactatatagtggaacagcaagaccctgtctgaacaaaccagcgtttggggaggctaggagtatagctcggtttgtcggatgcataccgcagagacctcaagacctgagtttggtccctagaactcaaggctgttactggtccatgtctgggatcccaactggacaggcagagacagaagatgcccgggtttatgggcagccagtccagcctgtttgatgagctgtaggctggtgagacagcatgtctcaaaggaggtagatagtgttcctgagatgcccccttgagattatcctctgactggcacatgtgtggcatctgcatacacacaaacctattagaaaccaaatcaaaagaggaaaaactataactattttctcttaaatataaggtaagagtgtatgttaattgcctctggctaattttttttttttttggttgctgagggcttgaatcttaaaatttgtctgctctgatttatttcatattattattattattattattattttagggacaggctcccagctgggtggtgatgtctcatttctttattctaagcacttgggaggcagaggcaggtggatctcttgagtttgtggtcagcctggtctacagagtgagttccaggacaatcagggctacacagagaaaccctgtctcaaaaaacaaaacagaaaaaaaaaaagacaggctctcagtatgcagcttagactacatatgtgaaactctgtccctcccccacataaaagtaaaaacagattataaatgataaattagatatcactattaaaactcctatttcataatacgtagtcaagactataagaagagaagccacaggatggaaggaagtggttgcacatcctctcataagctggtgtctgggatatagaaaacggagcgataaaacacagtcataaatacacagatagtgtactgagaacagggcagagggtctaagtgaatggtccttaaattaaatacatatgattgatagagaagctggtgaacacattcaggacatcggttaaaagctccacgtgctcttgcagaggacctgggttcagttcccagcactcacacatgcgtggtaactgcacttccagggtacccgatgccgcttctgacctccgcaggcaccgcacacacatggtgcacatctatccgtagatccaaaacacttgcacacatagaggaactcttaaaggcccagcatgctgaacagccacagtgtagttaaaatcacagtgccatactccctcatactcaccaggatggggataacgagaatatgggtaatatcttaaggagactatggagaaattggaaataccatacaactgtaagatggtgtaactgctgtccaaaaacagttcggcagtttctcaaaaaggtaaacatagagttaacatagaactcagaaaatccattcctcatttgatattatgattgttgcaaatatatcatccaaaagttcatacatagctgtagtgagagctttggtttctttggaaatgcttgggtttttttttttttttttttaagatttatttatttattatatgtaagtacactgtagctgtctccagacgctccagaagagggcatcagatctcattatggatggttgtgagcctgggatttgaactcaggactttttggaagagcaggcagtgctcttaaccgctgagccagtctccagcccggaaatgcttgtttttgttataatcccaagtgcccaccacagcagctgattgtgatttgccttgaactccagcaggagcctggttctgcaagctgtagctcgatcctgtgcttgggtagcgtggaattgtgggggctttttagaggatatataaatgttagcgctctgagccgtggagttgttgtcggttgttgggtggttgttgttggttgctgttggtctagctggttaagtagtcctatgcaaagaagaagaaattagatatcctgatcaaacttgccccaaggaactcagatgttcctaatgagcaggaagtagtctaacaatgacatcgccccctttcccctctaccctttcttccctcctgtcaagtcttagggggttgaaagggtggaaaaaaaaaaaaaagggtggagaagggtggaagaaagaacacaaaatagccaaagttgggctacacacactgtttaaaatagctagaaaacaggaagcaactcatattctctagctaacagataagtgaaacatagtcaacccatacagtggggtttgaaggtaacaaggagttgtgagttatatataaatacttgttcatgctattgggtaaataacccttgaaagcattgtgataaagaagatacaaaacaccttgtatcgtgtgtgttatctctctcatcttgctatgtacttctggctatctcccagtacttactatatagcccaggctagccttgaattcttggcagtttgttgcagcctcctgggtgctgagattataggtgggcaccactacacctagcttccttttttgacacacgtctggctaaaaatacaagctggccttgaactcactctgtagttctagctggcctccaatttataattttcctgtcttggcttgcctcaatgttgggattataagtgggtgtcagtgtgcttggcattgcaaggtttcatgtatatgaaatgtcctgttctgagtctcagatgttccctgaaggctcccatattaaaagttgggtgcccacagtgatgctcttgatacatggtggaacccttaagaggtgcagctctgtaggtcattggaagcatgcccttggtggggtggttactgtaggttgtagttggcatggccttgctagggtagctgctataggtcattggagggctttttctttttgtcttttccttcctcctaggatcatgatggaagtgacttccatgataatgttctgcctctccacagagctaaaaacaaagagtcccaaataaacctaccgacagacatcaaacaacgcatctgccaagttctgttctgaaactccaaaactatgagctccaataagcctcttctgtctatgttggttataatactcagacgtagtctcagagtttcagagggacagtgtgggagcctgcctcacatacatagatagttgctaaagctggccctaaagcagaggtccctctagttccagtgactcaggaggctcctcaggaggctgaggcaggaggattggttggacccaggaaacagcctgggtaacatagtgaggctaagtataaagtgttaaaatgaatgcactgttttgaataaggcatagatttccagtgttcagatccagaacctgctggtggatatcaaatacaaaggcttccatccttccctaactcccagccactcggtttctttccctggcggcagccggtgccatccagtgatgggaggaatcttggtatgccttgtaggtctttatttttagaagagtccaaaccctggtcctagctgcattctctggagtttacgtcctgttgggagggaggcgttgcagaggtcaggtgagggatggagctgagagagaaggctctgaaggcccttggggtgttgtgttgttgctttaattaattggttttggagagattatctcatgtacccaaggctaatctggagctcactgtgtaaccaaggatgaccttgaactttgatcctcctgcctctacatcctgtaccaccaggtctagcttgggggtggggggctggggggataagtccaatgttttatgtatgctaggcaagcactctagcaaccgagctacatcctatatccctagccctacttattactgtgtgtgtgtgtgtgtgtgtgtgtgtgtgtgtgtgtgtgtactgtagagaatcaaatccaagattggcatatgccagtgctctaccactgaccatccagccttgctttgtgtacactattctcatgcattgcttgcattattttttaaagacttcattttatgaatgtgtatatagtgtggcccggggcattgatgaggagtgcatatggaggtcagaggacagctttgtgcagtttgtgtgcctttttcatcttcatgtggaccccaggaattgaactcaggttgttggtctttaattcactgatccatctctatgacctcgttgctttcattattaatacttaattaaaactggtacccactgcagagggtagctataaggacacagttacagagcctgatgtgtgctaaagacatcactatgtgctaaagacatcactgtgtggtcctcgtttctttcatcatgagttagaagtacaaaggtctagggaattgcatcttggtgttttcacctgtgagatttttttttaaccacatctctttcctccagcaaaaaggaattggcatgaacgaaccgctggtggactgtgagggctatccccgggcggatgtggacttgtaccaggtccgaacagcaaggcacaacatcatctgtgagtgaccctcgctggggctgtcttggcttgtggtggggttgctggagacacagggggtgattagcattgagcactgcaaagcttttctgctttgacacaattaccttgtcaaagtaaggaactgagaagcaagcggacagggtggatggcagagttgcatgtcgatggctttattattatttttttttattttaaatactttgcttcaaagctaaatgtggtggcacacgcctttgatcccagcattcaggaggcagaggtagatggatctctgtgaggttgagtccagcctggtctacagagtgagtcccaggccagacagctgaaaaaaaaccctgtctcaaaacaaaacaaaacaaaaacaaacaacaaaaatccatgaacaaacaaacaaaaaaacttggcttcaagctgggcatggtggcacacagctctaaggctagctgtttgaggccagtctgggctgcatacaaaagtacagaacggggctggggagatggctcagtggttaagagcactcactgactgctattccagaggtcctgagttcaaatcccagcaacaatatggtggctcacaaccatctgtaacacaaccacctcttctggtgtgtctgaagaaagcaacagggtactcacatacataaaggatttttttaaaaaaagtaaaaaaacaaggaatgaaagctttactttgttatgggacttcattgaattaacaatggaagcccaaatcagtcactccccagccccatggagtccatgaagtaggttcccactgtgcaaatagcaaagacccaggctggctctcagactgaagtatcagtataaaggccagcagaaccaggttctgcctgtcttcaggcccatggctgtgttcaggcaggagacaggcgacaggaggaacggtcctggcctcccaccctgagtgtgccattggttagcattttcctgccaggaaggctgggcaagcctgtcccgctgcaggcccatcactctcctgagggagtcttacacatggtctttgtcttaatgaagtacctgttagagaatggcacctgcagaggacactgggtttgtgtttctgttataacaagccacaagactgtattgtgtctgacagttctggaggtgagacagccagaaggtctgctgacagaatccaggtgtcagtggggctggctccccctaggggctggggctggtggtgtatcctttgtctttgcagaggccacctgtgtttcctggctcttggccaggatcctctgtctttagaagtagtgctcacatctttctagcctttgcagctgttatggaatcttgtcctctccccctcctgcctccctctttagcttgttaagacccgggccctcccagaaaatccaggctcgtcccttctcccagggtcctgaggtcacatgtgcactggcttcgggatgagggtgtgaacatcttttacttggcactgtagataatcagccctccatcccagctgtactagggggtcagtcacccatggtttgcatttcatgatttccactgagtatgccggactccggctttatccacagtgttttggtaaaggagctgcctttataagcagggtgcagagtaagatgcacgctcccttaaagccacagtgtgggagagagcacctcagaacatcctttttcccctgaagattctgatgttaaaaaggttcagtcttacaggaaataaactgaggagatgcggagaatagggaaactatgtgaagactgcaaatgtgagggaaattctgagcttaaccccaaacccctgagcccccagggatggggctcaccccttctgcagaagcagggccggggaagtttgccgaatgctgattttcagtgctgattgacttcgtgtcccaacagcattctttccgctgacttgaactgcagcagtgttatattctctgtgtttgtgtgtgggtgtgtgtgggcgcacatgccctgctgggagcggaacctggggcttcgtgctagagcttactgtaccacttaggcttttcagctatgcttagcctatttctggacgctttacactgttctactccatgttaattttatatcatcttaatgactttattatgtgttaataatatgattgaacaattcccccttattagtattcttttgaaagacaattcccacaagtttaatgtattcttaaacatggatttttcttagggaacaatcctaggcttacattgaaattgggaaagtaatacagaaaggtctcatatgctgagtccttagtcgctaatcaccagcaagtgctagcatggtacatttgtggcagcttaaacttaattttgaaataattgctgcagaagtgtttacccccaggcaatgaaaaccttttgaattctgatatggaattgtaatatatcagcaactcagtcattggtcctctcacccacagcagtgtggagaatacagattgtgtctaagacagtgactctactgtaagcctccctggcctggggtctggtttttccccctcacatctgcctctgtggactgagtacccaagtcccctaatgcctgcgggccctacctttgctcacagtgacaggcaggctagatgaccacacagccacacagccccacagtcacggctcctgcagagttggagagtctgtgacccaagggtgccacatgtggattggctatcagggcgatggcagcaggcggccttggagactgcggagagttctattgatccagtggcctgggttgaataaggactgggatccaaagaagaaggaaaatatttttttttagttttaaaaggacattaaatgccggctgcttgcaggaccagtctccagacctgctggacgaccacggtgattcattttgttttcacttttatttacatgtataggtgttttgcctgcatacggtatgtgtgccaagtgcctgcaaaggccagaagaggacattagatcccctgggactagagttacagacagttgtgagcctctatgtgttgggaattgaacctgggtcctctccagtgctcttaactgctgatccatctctctagcccctgattcaatattataaatcagcttttagcactaaaacttggaggcagtctccttctcaaagtcctcagtttttagcctttctaggaaaatcaaagatttggcagcattaaatctgcctctcacgcctcatcagttgcaagagcccagtagtaggtgcctgactgacactcagcatcccacacgtggctcactaggctcttgtgtcttatccacctcctctatttaagagaaaaaaaaaaagacaggcttgtatgtatttgaggctaaccctaaacaaaatacttagctgagcatggccttgaactttttttttcttttaaagatttatttaatattatacataaatacactgtagctgtcttcagacgcaccagaagaggatgtcagatctcattacgggtggttgtgagccaccatgtggttgctgggatttgaactcaggacttttgggagagcagtcagtattctcacccgctgagccctctcgccagcccagccttgaacttttgatccttctgcttctgcctcttgagtgctgggattacaggcaggcaccaccacgtgagttttatgtggtgctggggatcaaacacctcttgtggggactcagcaagctctgccaacagagctacactgagctccagtgaaatccttccagcagacattcttcctcagccttgttttgtggtgtcttttccttgagagtcttaagtacctaacactggtctcctgatctcctcctgcctcccctgatctcctcctgcctccccctgatctcctcctgatcctccccctgtctcccaggtgttgtgattgcagatgtgcagcatcctgcccagccacttggcttttgtgtgacttctgtggttgtcccttatttgtatttgaggtcagggcatagttgcagctggatggaacctgatttctactcacctttcaggtctcagcaggggctgtgctgcatttgaggggccttcttgctgttctgaattatccggtcatccgatgtgattatattatagtcacctgttctgtagctgccttgctcactagacttcctcactagtgaggtaggccctgtgtctggctcacagacttctctgttcctcagactctggctttgtgtctagcacctaataagcacttcttaaatattctgatgtgttagttcctttttggttgctgtgacaaaatacctgataaaagaaacttaaggccgggcatggtggcgcatgcctttaatcccagcacttgggagacagaggcaggcagatttctgagttcaaggacagcctggtctacagagtgagttccaggacagccagggctacacagagaaatcctgtctcgaaaaacaaaaaacaaaacaaaaatatatatatatatataaagaaaaagaaaagaaacttaagaaggaagtttggctcacaggggcagaacaaacttcccactggagaagacgtgatggtaggagcctcaggtagctggttagagagagggtcactgcagcaggaggcgaagagttgaatgttggtcctgtctcgtgggatggtgccgcccgcattcagggaggactgttctccttcagttcagcatctctggaaatgtccttccataagcttccaggggctgggcttggacagttctgaatctcatgaggttgacaacatacaccattgcagcgagtgaattcagctgtcccagctcctctgtcctgtgtcccgcgctagcttcctgtgccctgacatggtgttctctctccaggtctccagaatgatcacaaggcactgatgaagcaagtggaggaggccctgcaccagctgcacgctcgggacaaagagaagcaggctcgggacatggctgaagcccgagaagaggccatgaaccgcaggctggcctccaacagccccgtcctgccccaggcctttgccagagtgaacagtatcagccccggttccccagccagtattgcggtaatcttgctggctctcccaaatccactcctaacagtactgagagttaggtagccctgggcttcggaggcctgtcagaaggcaaacccgtcctctcattctgtggctctgaattcctttattcctttaacacatgctggccagagcttggtgtgcataggggcagcccacggactcagggagagacctatggtgggaccatctgggacacagtgtagaaagtgatccatgccccaaagaagaaaagaggtgttcttcttggagccattttcctggacacggaaagaaaaaaaaatgacaagagcttaaagcgatgtcagtgttctttgatagatcctagagcggcattactgtgatgggctgctcctgccatgtgtgacccaccaaaggacacagaggagtcacagagctgcacatttagcaccgcagccaatttcacagtatttttattggtgctgaaaagaagccaggcaggacttgttgagcaccagtaattttgtctgcacagatctgcctgttctgtgtctttgatgtgaagacagctgtatgctgtgtgctctctcactcggtctgttcccagggttcactcagggccgggtcacagatgaagccttcattacgttttatggctgagcaatatccattatgtgataggcatgttcttgttggtcagttaatggatgtctggatctcttctgctttggggctattatacgtaatgcggcagtgaatattcatgtagaagtttgtatgtggctgtgcatggatattgaataccgtggctgctgtgggtggcaccactgctctccgtgtgtgaccctcgaggacaagccacacatggctcctgggaagcacaaatggattcttttctttctttttttctttagtattttgaaacagggtttctctgtgtagccctggctgtcctggaactccatctgtagaccaggctggcctcagactcacagacatccacctgcctctgcctcccccattaaagacatgtgtcaccagacagaactggattctgaattcggatcctacaaatcatcccaagactgggggataaagggatggctcagttgttaagagcactgactgctcttccgaaggtcctgagttcaaatcccagcaaccacatggtggctcacaaccatctttaactagttttaggggatccagttctctcttctggcctccatgggcaactgacatacaagttcacagacactcaagcaggcaaaatgcccaaacacattagataataaaataaaataattaaaaaaaataatcacaagcttaaaaacggtcacaaggccactgggtatggtggtattttttcataatcccaactactgaggcagctgagtcaggacttacccaagtttgggcaacttaggaaaattgtgttttgaaatacgaaagactgaggatgtagctgttgggtagagctcttgcctgggttggattcctactgctgtgaacaatttttaaaaagcaaaataaagtcaagacataggactacagactgcacgtcaactctagtcagagctggattcgcaacccccagcgcctgactagtcggcctctggtgggtgtttgttaaagagaccagtaagtagtgtctgtaatcttgggatctgaaataaaaggaccacactggttctggctggccttaggcgtatggctgctgcttagggactcggaggaccaatgtttgtcttctcaaaaaaaaaaaaaaaaaaaaaaaaaaccaggcctaggctaaactgcctccttgccttgaacctgcctcagaagctgcagaacacagctcatgtcctcaaggtgtccactgggtacacctggacaccatttgctaggtctgtcctagaaggaacctccagaatgaggcacctgcctactccctgatcctactcgaggagttgaaatctttgacctcgttacttagagcctgccattctttccctgtaggaaacaagctcttggtcttgccacagagttgtagattaagcggtacaaatgaccagaaaatgcatccaacctctcgggttggctttacatttttctcctgtctttagggcctgcaagtggatgacgaaattgtggagttcggctccgtgaacacccaaaacttccagtcagtgcagaacgtgggcactgtggtgcagcatagcgagggggtaagtcggggtgccctggcatccgggtgtcccacaagccacagaggcctgctgctcgcacagatcaggggatagtgtggaagtccaggagcagggggccacagattcaggctctggtccaaggatgacacagtttgcttaagggctggatcactgactccatcaccagggctccatccccataaattgcacccctctaaagcccagtctcctactccatcacaccgaggggccagtcagccagcctttcagtatgagagctttccagagggaagcaggagttcagcccatagcgctgggtatctgtctgtctgtctgggattttccggcccagaagctgttggggggatcctcactgctgacagcagtggtggtgcctctctctctccctggagagctttttcttccatttgccacatttgtcttacacagctgagtcttttacacagttgatggccctcctggtgctcttgtctaaatccctcctctgtgtcatccagtaacagtaactaagctagaagctgccttattttggcacatgccctgttcccctgcagctgttattggggtccagttctccatcactgccttctctagaccttgtgatgtttccagcttcacccagggtggccgaccacgtacgtgtgcacgtggtcacatgcttttctaggtggcacctgtaaagaagggtggccggctctgagagatcgtgtatacttgaggcgtcttattctgtctcaggctggcctggatctcactgtgtggcctaggatgatcttgagccttctggttgtccttcctcccgtgcactggaagtacagtgtgcctggccatgcctgtttttatgtaatacggagctatctctctagcccgtggatataggttcttctttttctgacacatatatttcccaggaatttattctataattagtttagtgtaattagacacacattaccaggccacttttccccccaaatggatagtagttgtcccggtaccatttatcattatgaatactttacccactagtttacactgtcacctctgttaatatcagatatatgggtggtgttctctttgttctgaccctttttggagttgcttagaagaatccatttgtccctgtctgctttcctttaacaccttgctatagagtgagtccaaggaccatttggaaagttggtaggccaagaccacagatgcttgtctgtctggtacagtgggctctcaaccttcctaacgctgccaccctttaatacagttcctcatgttgtggtggccccaaccataaaattacttttcttgctacctcatgactgtactttggctactgttagaaacagtaatataaacatctgtgttttcagatggtctcaggggagccctgtgaagtgggtgtctcaccccaagaaggttacaacccacaggttgagaaccactggtctcgttggacagctcagccatccctgctggctctgctggttctggcccagaggcttctgtgagggccagccaggacacctgcagacaccatggccacccaaagggtccagcgaggctgtacatccacttccaccccatggtcggcccgagccttcagttactcatcctgcggcttcatacaccacttgagtgcactacctcaggcagcgagagcagtgagggggtcgcatctcagaagatgcacagttatgtctttcttacctcttagtgacttgctaggtccagcccacgcctggggagaagggaattggacctcctgaagggaggcatgtgagaggacttggggcctatcattaaactgccctccgagggctccagcctctctactcgccctgccctcacttgtccagtttgtggaagctttccttgtgttagcggtccctcatctctgtcatgtcttcccagtgtttgtgtctgtggctgtccagccttgctagcctgctgttcttcatcatctcagtgacatcatctctgtgacagtgcgctgccacgcccagtgtgcgtatttgcagatgtgtttttaaagatgtcctgttacattctgaattcagacccatggaaaacagaaccaaagcacaaaattggcacaaagtcttcaggttcgtttcctggcttccttttagctttttttcaaaagacttactttggggagtcccaatgtgcccgctgtctaaaagccaagtggccatggtaatttgaaagccacaccgctgatctcagtcctgaccctcagggccctgcttgagagtcaggagtctgggttctcatcgtctcttctgtgacttaaacaaaacaaaaccccaggtctgtctgtgctgttagcatttcggaagtttccaattttattacgattctgcttttgtagagcaaactgttttcatcagctttccttccaaaagtatagtgtttggatcaaacgcgtatattctcaaagcgcaagcaatcttgatgggaaaaaaatttactgtcttgaccctaagggtgtgtggttcagtggcaggtcccttttcaggtgcccagtcatgggtttggtcaacaaaagtgttgttgttgttgttgttgttgttgttgttttgcaacaacaatttctgtctgccctggctgtcctagagcacactatgtagacagactggcctcacagagatccaattacctctgcctactgagtgctgggattaaaggggtgcaccaccatgcccagcacactgttttttgggttttggtttttatttatttattgtttgtttgtttgtttgtttgtttgttatagccctggctgtcctggaactcactttgttgaccaggctgcccttgaactcagaaatctgcctgcctctgtctcccgagtgccgggattaaaggcgtgtgccaccacacccagcttttggtttttttaaatacatgtgtgtgcacactcgcatgcacctttataataaaaacaatgcatatttagtttagccacaggaatggagtaaattctccattacatattacaacatgagtattgaaaacataatataaagtgaaagagaccaaacaggaggccatacaattcctcaacgaagttgtcctaaaaagcagatctagaaagctagaaaggagattggctggtggttgctgggcgggagggtgagggtgaggaccatgtgctgatggtcttttggatgatgcagacagaccctctgcagttaggaggtggtgatgggtctgagagcaggagacccactgagccctgctttactgtatataaattgtgtctcagtaagaagcctactgcatatttatgtcacatacaaattcacattgcaaaaatgtttaaagtgaaaatcaaagcactgtgtctgtctctctctgtaactccctcccctgccctcccctgccctcccctgccctcccctgccctcccctgccctcccctcccctcccctgccctcccctcccctgccctcccctccccatgtgtgcttacttctggactgagggctggggtccagcattgaacaaaaatcaaagagagggaccttgctttgagagcagacatgagttcagtagttccggacgaccaattttatatcctgaccagagttgacaggagaagccttcgcaggagagctggaggagagcagtggggtcctggtgtagtttggggtagaaggagtgttcctaagtactgaccctcggagtaaggactgacccttggtgtgtatagaaactgatctgtggagtggccactagagagcgcccttcagctggttctcaggccttggaggcaggaggatcaaagcagccagattgttgattggtgctcagaaccatgaatcagggaggtgaccagggccaggccatgcagggccagcctcggagtcccttacaaagaaaatgatgacttttttttcctggggagcagcagggagctgctgtaccacccaaagctggggagagtttgtctcagagattgcatggtgctagtgagagaacaggtttgggggtgtgtccagcatgtgggtctgtgtcctcatagggtctcaagtaggaatcaagtgtcagatgccggaacttaaggagacagttctcattcaagtcgacatagttcctaccatgcttttaccctcccaccatttttaatttcttcatggttcattaatgctggggcccaaagtggtcaatttcatggtcagaaaatggtatctccacatactgagtcctcttgagtggaaggaagcagaggctggaatgaccttctttgcctttcagaagcccctgaatgtgacggtgatccgcagaggagagaagcaccagctcagactgattccaacccgctgggcaggaaaaggactgctggggtaatgctgggctctgcttcccatgctctcacaccctggcacccacagagagtgcgctcagatagagctggggatgctggtacccatagagagtgtgctcagatagagctggggatgctggtacccatagagagtgtgctcagatagagctgaggatgctggtacccatagagagtgtgctcagatagagctggggatgctggcacccatagagagtgtgctcagatagagctgaggatgctggcacccatagagagtgtgctcagatagagctggggatgctggtacccatagagagtgtgctcagatagagctggggatgctggcacccatagagagtgtgctcagatagagctggggatgctggcacccatagagagtgtgctcagatagagctgaggatgctggtacccatagagagtgtgctcagatagagctggggatgctggtacccatagagagtgtgctcagatagagctggggatgctggcacccatagagagtgtgctcagatagagctgggaatgctggtacccatagagagtgtgctcagatagagctgaggatgctggtacccatagagagtgtgctcagatagagctggggatgctggtacccatagagagtgtgctcagatagagctggggatgctggtacccatagagagtgtgctcagatagagctgaggatgctggtacccatagagagtgtgctcagatagagctggggatgctggcacccatagagagtgtgctcagatagagctgaggatgctggcacccatagagagtgtgctcagatagagctggggatgctggtacccatagagagtgtgctcagatagagctggggatgctggcacccatagagagtgtgctcagatagagctggggatgctggcacccatagagagtgtgctcagatagagctgaggatgctggtacccatagagagtgtgctcagatagagctggggatgctggtacccatagagagtgtgctcagatagagctggggatgctggcacccatagagagtgtgctcagatagagctgggaatgctggtacccatagagagtgtgctcagatagagctgaggatgctggtacccatagagagtgtgctcagatagagctggggatgctggtacccatagagagtgtgctcagatagagctggggatgctggcacccatagagagtgtgctcagatagagctgggaatgctggtacccatagagagtgtgctcagatagagctggggatgctggtacccatagagagtgtgctcagatagagctggggatgctggcacccatagagagtgtgctcagatagagctgggaatgctggtacccatagagagtgtgctcagatagagctggggatgctggtacccatagagagtgtgctcagatagaacactcaggtccactaaaggatagccatccccagagtgcagaaactggtggttaataaccgtttttatgaatgaggctacaggcaagagtcctcaggtcacctgagagtctcacttctcaatccctgggcattgtctttaagagaaagaaaagtgaccagtgctcagcaggtaaaggtctttctgcaaatctctgacctgagtcagatcccaggactcacttggtggaaggagagaactgactcctgcaggctctgcgtgccacacacccagactctcttatatacacaatgaaacactgtaaaatatacattaaaaaatacaattctagccgggcagtggtggcgcacgcctttaatcccagcactcgggaggcagaggcaggtggatttctgagttcgaggccagcctggtctacaaagtgagttccaggacagccagggctacacagagaaaccctgtctcgaaaaaacaaaaaccaaaccaaaccaaaaccaaaaccaaacagtctttaaaagagaagaagtgttcacctgcaggtttcctaggctggagctgtcaggcttgagccggtctctgttttctcttacagctgcaacattattcctctccagagatgactgtttcctgggatctgcctgcaggaagctgcctcagctggccccgtgcttgggcctggaggcgtttcctcgttctctaggctcccttaagtgtaaggatctggagaagaatggtcgaagcctgggcatcgaggtggaagagacgctttggctgcctgatgtaatctctctgggttgaggcattattaaaagtgtgatttgtgcctagctacgtcttgtgcaaattaggccatagcctgtctgggaattctctagattatgagccagtgagtggggatgactctggcgagtaatgatttgatgtcattttccttttggagacggactcagtgtgtaatcctgactatcctggaattccctgtgtagaccaggctggccttgagctgacagagatccccctgcctctgcctcattagtattgggatgagaggcatatactgacatggctggctagactctgaggaatatgtattatacatatataatttatacttatttttgagaatttcgtacatgcacatagtacatttctatatttcctgtcatctcaagtccctctcagacctcccctcgtacccacttcccaggttcttgccctcttttgttttttttctttttaaataaataacctactaagtgccatttgtgctgtcagtatattcatggtgtgggatcttccactggaggaatgtggtcaaccttccagagaccacacccctaacctccacaggacttgagtacccctactttttcttggaagcagggtctcatgtagcccagtccagcctcagatttgccttgaagctgaagatgttctcttctgctgtctctccctccaatgtccaactgcagtatcacccaccgcctaggggtgggggcttgctggagaccggatctcacaagttcagactggcctggagctcagtgtacccgcactggccttacccttgggatcttgctgccctggtctcctggtattatagtgtatgccaccaaaccttgttacaggttagagaaactgtcagataaagccagtcttgtagctgtgtgattgtgaccactgtcagtcccaccaggatgccctgtgagctcacagccaagcccaggaggagttgggaaggaagtcctgcgaagcaaacgagttcctgggccgcagtccggttgattcttgggtgggaaactgaaagccggagctcagactctaggcaagtgtccctccgtgggctgagcagctggcagtctctaccctggagcagtgctttgagagcctgatgcatcctctgaggctgaggctgtggagtcaccgtagccccattaagaaatcagcaaagaccaggtgtggtggctcaaacgttttaatcccagcactcaggaggcaggggcaggcggatttctgagttaaggccagcctggtctacagagtgagctccaggacagccaaggaaggctacacagaaaaattctgtctcaaaacaaaatttgcaaaggtcacaggtcacgaggccttgcaggttacccagtatgatattggcttcctcttcagtaacccactggaaaccagcaagcttcctttacttctacagtaaaagaccccgcagatgggcctgatttggcttttatagttgagcctccccaccgggcggtggtggtgtacacctttaatcccagcactcaggaggcagagagaggcaggcagatctcagagtttgaggctagcctggtctacagagtgagttccaggacagccagggctacacagagaaaacctgtatggaaaacaaaaataaaacaaatttaaaaaacaaacaaaattgagcctcttgggatcagcatgggaagctaaggagtgcttgcctgttgctgaggtgactgcattgatggatgctgctttgctgcttcctttcatattctgtcttttgagatggtaaaattggtgtgggtgactctgtttttcttcctttctccatgtgccccagttatctccccagttgcctcccgtttctgttttggatgtctggtcctttctacaaggaagcttgcgtccttgcattggatttccgcgggatggaggcagagaccagctttgtctgatggcagtcagtactgacatgtgcttcctgctttgagtccgcacacacaactcaaaagcctcccctaagctgcttcatccagacagcctcacgccacccctctcccgtagccagactggctctgtttactagggtttgctgtttcagttgtgggttataaatccaagtattttccagactctttataagtctcacactgtaataaaacacagcttgtaccattcaagagataggcatgtgatcaccagcactggggcgtggtgtggtggtggacttgtccatagtcattcatatcctctctaagaggctgcaataggccttgcactgctcagaaagccacctttgggctcccctatgacttgcttcaacaggaataacgctaatggatttttttttttttttttttaaagatttatttattattatatgtaagtacactgtagctgtcttcagacactccagaagagggagtcagatcttgttacggatggttgtgagccaccatgtggttgctgggatttgaactctgaaccttcggaagagcagtcgggtgctcttacccactgagccatctcaccagccccaacgctaatggatttaaccaagctgggcttgcgtggtaacagcaagatggtgcactctccacataacttcaatcccagtttactaaacttctctgcatgctgtgtcctggtgagaaggtgggcagttgtgtgtaacacctgtgaggccaaaaaaattgggttttagcactgggagaattttattaactgcttcataccacttcttgcctggcttcctggagaggcatgagcacacatctgttcaccttgcactgatggcaaactgaaggaacccacccaaatcagccctgtgagcccgtgagcctcctggggttgcttagaggctctctcggcactatggatatacaactcgcaggcagctagctccactgaaatctcctttcacacagtaaagtcttgtgtagtccaggctgactgaacagacccttctggcttcatctgcctagttctggatttgtaggtgggacatcatgcctggcccaacgtctagttttttgttttgttttgtttttaagatttatttatttatttattacatgcaagtacactgtagctgtcttcagacgccccagaagagggagtcacatctcattacggatgattgtgagccaccatgtggttgctgggatttgaactcgggaccttcggaagagcaatgagtgatcttaaccactgagccatctatccagcccccaacgtctagtttttatttatgtatttatttattcattcaatcatccgttcattcatttatttattttactgcttttatgacctccggaagggcttcctgacacttgagggaaccccaccacccaacccccaccgagttttttgagcaggtctccgtttcccctcccctacgaatgcccaatctggagaaaataactgcaaacactactttcgttacatcttgtccaactttaggaaagtcagaatcgccagtgtaggggagtagctcttgtggaagtgtcctttaggaaaaagctgtggcgtgagctgctagcccggcagggggcaggctggtccgcctcgaggccgctttctgggcacgtgattaccatcctgcttccttgctttcctgcgactagccgttgccatagggacggagcttggttggcaaccgtcactagggagtatggacaggagcccctggaggtctcttgggatttcccgaggctggacgacccaaggcttttgagaaaagaagtgggagcgtccctgtaagtgcgaagtggagggtgcggctcgggttggggtccgggccaggcagcggcaagggtgtgggacctgggaggtccccaggaaaagaaactggtggctcctggacctcgtggtaggatagacttgcgcacctgtggcattgcccccgcctgggtgtggaaaatgtcaaagtggcatttttttcaaacaaaagaactaaattctagctctgtcgtaagtcgtgcctcccagggccacaaactttttcatttccttttccttttccttttccttttccttttccttttccttttccttttccttttccttttccttttccttttccttttccttttcctttccttccttccttccttccttctttctttctttctttttttagaaagattgtgctggcctaagaactcgagtgatcctcctgtctctacctcctgaatcgcagtattacagacatgggcctagccccctgctcccaaactcttgcttcatgtaggtgcaaaatgagagaatgtatcagaggaattaaatttttttaaaaaatgattttatgtgtgagactgttttgtctgcatgtatgtatgtgtaccacctatgaagttcccgcagaatacagaagagggtgtcagatcccctgaaactcaagttacttatggttctgagcgatcgtgtcatggctgggagtcaaacctgggtcttctgaaagggcagccagagtgctcttaatcactgagccatctctccagcccagaattaactttctttctttctttctttctttcttt

SEQ ID NO: 12 - DNA, 19 bp, Artificial Sequence
  Primer
tgatccgcagaggagagaa

SEQ ID NO: 13 - DNA, 22 bp, Artificial Sequence
  Primer
gatcccaggaaacagtcatctc

SEQ ID NO: 14 - DNA, 24 bp, Artificial Sequence
  Probe
aggactgctgggctgcaacattat

SEQ ID NO: 15 - DNA, 16 bp, Artificial Sequence
  ASO against PSMD9
gcaagtacggaaacag

SEQ ID NO: 16 - DNA, 16 bp, Artificial Sequence
  ASO against PSMD9
ggctacgggtcctccc

SEQ ID NO: 17 - DNA, 16 bp, Artificial Sequence
  ASO against PSMD9
tcggaggactctgccc

SEQ ID NO: 18 - DNA, 16 bp, Artificial Sequence
  ASO against PSMD9
gctgaccgcggccgca

SEQ ID NO: 19 - DNA, 16 bp, Artificial Sequence
  ASO against PSMD9
cgtaattagccttgat

SEQ ID NO: 20 - DNA, 16 bp, Artificial Sequence
  ASO against PSMD9
gatgatgttgtgcctt

SEQ ID NO: 21 - DNA, 16 bp, Artificial Sequence
  ASO against PSMD9
agcctgcggttcatgg

SEQ ID NO: 22 - DNA, 16 bp, Artificial Sequence
  ASO against PSMD9
ggctgatactgttcac

SEQ ID NO: 23 - DNA, 16 bp, Artificial Sequence
  ASO against PSMD9
aagttttgggtgttca

SEQ ID NO: 24 - DNA, 16 bp, Artificial Sequence
  ASO against PSMD9
tggaatcagtctgagc

SEQ ID NO: 25 - DNA, 16 bp, Artificial Sequence
  ASO against PSMD9
cacttaagggagccta

SEQ ID NO: 26 - DNA, 16 bp, Artificial Sequence
  ASO against PSMD9
gcccaggcttcgacca

SEQ ID NO: 27 - DNA, 16 bp, Artificial Sequence
  ASO against PSMD9
gagattacatcaggca

SEQ ID NO: 28 - DNA, 16 bp, Artificial Sequence
  ASO against PSMD9
ggcacaaatcacactt

SEQ ID NO: 29 - DNA, 16 bp, Artificial Sequence
  ASO against PSMD9
cctaatttgcacaaga

SEQ ID NO: 30 - DNA, 16 bp, Artificial Sequence
  ASO against PSMD9
atctagagaattccca

SEQ ID NO: 31 - DNA, 16 bp, Artificial Sequence
  ASO against PSMD9
tcattactcgccagag

SEQ ID NO: 32 - DNA, 16 bp, Artificial Sequence
  ASO against PSMD9
catcaaatcattactc

SEQ ID NO: 33 - DNA, 16 bp, Artificial Sequence
  ASO against PSMD9
atactaatgaggcaga

SEQ ID NO: 34 - DNA, 16 bp, Artificial Sequence
  ASO against PSMD9
agtatatgcctctcat

SEQ ID NO: 35 - DNA, 16 bp, Artificial Sequence
  ASO against PSMD9
tagtaggttatttatt

SEQ ID NO: 36 - DNA, 16 bp, Artificial Sequence
  ASO against PSMD9
aatatactgacagcac

SEQ ID NO: 37 - DNA, 16 bp, Artificial Sequence
  ASO against PSMD9
tggaagatcccacacc

SEQ ID NO: 38 - DNA, 16 bp, Artificial Sequence
  ASO against PSMD9
gggtactcaagtcctg

SEQ ID NO: 39 - DNA, 16 bp, Artificial Sequence
  ASO against PSMD9
cccctaggcggtgggt

SEQ ID NO: 40 - DNA, 16 bp, Artificial Sequence
  ASO against PSMD9
ggtaaggccagtgcgg

SEQ ID NO: 41 - DNA, 16 bp, Artificial Sequence
  ASO against PSMD9
tggcatacactataat

SEQ ID NO: 42 - DNA, 16 bp, Artificial Sequence
  ASO against PSMD9
agctacaagactggct

SEQ ID NO: 43 - DNA, 16 bp, Artificial Sequence
  ASO against PSMD9
atcaaccggactgcgg

SEQ ID NO: 44 - DNA, 16 bp, Artificial Sequence
  ASO against PSMD9
gctcagcccacggagg

SEQ ID NO: 45 - DNA, 16 bp, Artificial Sequence
  ASO against PSMD9
gggtaacctgcaaggc

SEQ ID NO: 46 - DNA, 16 bp, Artificial Sequence
  ASO against PSMD9
caatatcatactgggt

SEQ ID NO: 47 - DNA, 16 bp, Artificial Sequence
  ASO against PSMD9
aagttaatgcttccga

SEQ ID NO: 48 - DNA, 16 bp, Artificial Sequence
  ASO against PSMD9
cgtcatctggcaccca

SEQ ID NO: 49 - DNA, 16 bp, Artificial Sequence
  ASO against PSMD9
gccgatggtagtgcac

SEQ ID NO: 50 - DNA, 16 bp, Artificial Sequence
  ASO against PSMD9
tgcatactgagagcct

SEQ ID NO: 51 - DNA, 16 bp, Artificial Sequence
  ASO against PSMD9
agttacaccatcttac

SEQ ID NO: 52 - DNA, 16 bp, Artificial Sequence
  ASO against PSMD9
ggcaagtttgatcagg

SEQ ID NO: 53 - DNA, 16 bp, Artificial Sequence
  ASO against PSMD9
gtatttttagccagac

SEQ ID NO: 54 - DNA, 16 bp, Artificial Sequence
  ASO against PSMD9
tgtttgatgtctgtcg

SEQ ID NO: 55 - DNA, 16 bp, Artificial Sequence
  ASO against PSMD9
tccagattagccttgg

SEQ ID NO: 56 - DNA, 16 bp, Artificial Sequence
  ASO against PSMD9
gtccttatagctaccc

SEQ ID NO: 57 - DNA, 16 bp, Artificial Sequence
  ASO against PSMD9
cgacatgcaactctgc

SEQ ID NO: 58 - DNA, 16 bp, Artificial Sequence
  ASO against PSMD9
gctatttgcacagtgg

SEQ ID NO: 59 - DNA, 16 bp, Artificial Sequence
  ASO against PSMD9
ttatctacagtgccaa

SEQ ID NO: 60 - DNA, 16 bp, Artificial Sequence
  ASO against PSMD9
agcgactaaggactca

SEQ ID NO: 61 - DNA, 16 bp, Artificial Sequence
  ASO against PSMD9
tgaatcaccgtggtcg

SEQ ID NO: 62 - DNA, 16 bp, Artificial Sequence
  ASO against PSMD9
ggctcctaccatcacg

SEQ ID NO: 63 - DNA, 16 bp, Artificial Sequence
  ASO against PSMD9
cacagtaatgccgctc

SEQ ID NO: 64 - DNA, 16 bp, Artificial Sequence
  ASO against PSMD9
tgaatattcactgccg

SEQ ID NO: 65 - DNA, 16 bp, Artificial Sequence
  ASO against PSMD9
gcgaatccagctctga

SEQ ID NO: 66 - DNA, 16 bp, Artificial Sequence
  ASO against PSMD9
gcaaactgtgtcatcc

SEQ ID NO: 67 - DNA, 16 bp, Artificial Sequence
  ASO against PSMD9
ggctcaagatcatcct

SEQ ID NO: 68 - DNA, 16 bp, Artificial Sequence
  ASO against PSMD9
tggatgtacagcctcg

SEQ ID NO: 69 - DNA, 16 bp, Artificial Sequence
  ASO against PSMD9
cacattgggactcccc

SEQ ID NO: 70 - DNA, 16 bp, Artificial Sequence
  ASO against PSMD9
aggaattgtatggcct

SEQ ID NO: 71 - DNA, 16 bp, Artificial Sequence
  ASO against PSMD9
gggtggtacagcagct

SEQ ID NO: 72 - DNA, 16 bp, Artificial Sequence
  ASO against PSMD9
cagctctatctgagcg

SEQ ID NO: 73 - DNA, 16 bp, Artificial Sequence
  ASO against PSMD9
gggtaccagcatcccc

SEQ ID NO: 74 - DNA, 16 bp, Artificial Sequence
  ASO against PSMD9
ctatgggtaccagcat

SEQ ID NO: 75 - DNA, 16 bp, Artificial Sequence
  ASO against PSMD9
cacactctctatgggt

SEQ ID NO: 76 - DNA, 16 bp, Artificial Sequence
  ASO against PSMD9
tgagcacactctctat

SEQ ID NO: 77 - DNA, 16 bp, Artificial Sequence
  ASO against PSMD9
gctctatctgagcaca

SEQ ID NO: 78 - DNA, 16 bp, Artificial Sequence
  ASO against PSMD9
gggtaccagcatcctc

SEQ ID NO: 79 - DNA, 16 bp, Artificial Sequence
  ASO against PSMD9
atgggtgccagcatcc

SEQ ID NO: 80 - DNA, 16 bp, Artificial Sequence
  ASO against PSMD9
ctctatgggtgccagc

SEQ ID NO: 81 - DNA, 16 bp, Artificial Sequence
  ASO against PSMD9
cactctctatgggtgc

SEQ ID NO: 82 - DNA, 16 bp, Artificial Sequence
  ASO against PSMD9
tgggtgccagcatcct

SEQ ID NO: 83 - DNA, 16 bp, Artificial Sequence
  ASO against PSMD9
gtaccagcattcccag

SEQ ID NO: 84 - DNA, 16 bp, Artificial Sequence
  ASO against PSMD9
atgggtaccagcattc

SEQ ID NO: 85 - DNA, 16 bp, Artificial Sequence
  ASO against PSMD9
gttattaaccaccagt

SEQ ID NO: 86 - DNA, 16 bp, Artificial Sequence
  ASO against PSMD9
acgcgcaagtacggaa

SEQ ID NO: 87 - DNA, 16 bp, Artificial Sequence
  ASO against PSMD9
tgaggctacgggtcct

SEQ ID NO: 88 - DNA, 16 bp, Artificial Sequence
  ASO against PSMD9
cctcaagctagagttc

SEQ ID NO: 89 - DNA, 16 bp, Artificial Sequence
  ASO against PSMD9
ggcctcggaggactct

SEQ ID NO: 90 - DNA, 16 bp, Artificial Sequence
  ASO against PSMD9
ctggatgtcgctgacc

SEQ ID NO: 91 - DNA, 16 bp, Artificial Sequence
  ASO against PSMD9
tctctttgtcccgagc

SEQ ID NO: 92 - DNA, 16 bp, Artificial Sequence
  ASO against PSMD9
ggccagcctgcggttc

SEQ ID NO: 93 - DNA, 16 bp, Artificial Sequence
  ASO against PSMD9
cgcaatactggctggg

SEQ ID NO: 94 - DNA, 16 bp, Artificial Sequence
  ASO against PSMD9
cacggagccgaactcc

SEQ ID NO: 95 - DNA, 16 bp, Artificial Sequence
  ASO against PSMD9
cgctatgctgcaccac

SEQ ID NO: 96 - DNA, 16 bp, Artificial Sequence
  ASO against PSMD9
ttggaatcagtctgag

SEQ ID NO: 97 - DNA, 16 bp, Artificial Sequence
  ASO against PSMD9
tacacttaagggagcc

SEQ ID NO: 98 - DNA, 16 bp, Artificial Sequence
  ASO against PSMD9
ttccacctcgatgccc

SEQ ID NO: 99 - DNA, 16 bp, Artificial Sequence
  ASO against PSMD9
agagagattacatcag

SEQ ID NO: 100 - DNA, 16 bp, Artificial Sequence
  ASO against PSMD9
cgtagctaggcacaaa

SEQ ID NO: 101 - DNA, 16 bp, Artificial Sequence
  ASO against PSMD9
ggcctaatttgcacaa

SEQ ID NO: 102 - DNA, 16 bp, Artificial Sequence
  ASO against PSMD9
ataatctagagaattc

SEQ ID NO: 103 - DNA, 16 bp, Artificial Sequence
  ASO against PSMD9
aaatcattactcgcca

SEQ ID NO: 104 - DNA, 16 bp, Artificial Sequence
  ASO against PSMD9
actgagtccgtctcca

SEQ ID NO: 105 - DNA, 16 bp, Artificial Sequence
  ASO against PSMD9
cagtatatgcctctca

SEQ ID NO: 106 - DNA, 16 bp, Artificial Sequence
  ASO against PSMD9
gggacttgagatgaca

SEQ ID NO: 107 - DNA, 16 bp, Artificial Sequence
  ASO against PSMD9
cacttagtaggttatt

SEQ ID NO: 108 - DNA, 16 bp, Artificial Sequence
  ASO against PSMD9
tgaatatactgacagc

SEQ ID NO: 109 - DNA, 16 bp, Artificial Sequence
  ASO against PSMD9
gtggaagatcccacac

SEQ ID NO: 110 - DNA, 16 bp, Artificial Sequence
  ASO against PSMD9
tgatactgcagttgga

SEQ ID NO: 111 - DNA, 16 bp, Artificial Sequence
  ASO against PSMD9
ctgaacttgtgagatc

SEQ ID NO: 112 - DNA, 16 bp, Artificial Sequence
  ASO against PSMD9
tcccaagggtaaggcc

SEQ ID NO: 113 - DNA, 16 bp, Artificial Sequence
  ASO against PSMD9
tgtaacaaggtttggt

SEQ ID NO: 114 - DNA, 16 bp, Artificial Sequence
  ASO against PSMD9
tcgcaggacttccttc

SEQ ID NO: 115 - DNA, 16 bp, Artificial Sequence
  ASO against PSMD9
cccaagaatcaaccgg

SEQ ID NO: 116 - DNA, 16 bp, Artificial Sequence
  ASO against PSMD9
catcaggctctcaaag

SEQ ID NO: 117 - DNA, 16 bp, Artificial Sequence
  ASO against PSMD9
catactgggtaacctg

SEQ ID NO: 118 - DNA, 16 bp, Artificial Sequence
  ASO against PSMD9
ccaatatcatactggg

SEQ ID NO: 119 - DNA, 16 bp, Artificial Sequence
  ASO against PSMD9
ttttactgtagaagta

SEQ ID NO: 120 - DNA, 16 bp, Artificial Sequence
  ASO against PSMD9
gcgattcccgcactca

SEQ ID NO: 121 - DNA, 16 bp, Artificial Sequence
  ASO against PSMD9
tatgatggccagtgcc

SEQ ID NO: 122 - DNA, 16 bp, Artificial Sequence
  ASO against PSMD9
ggtctctgcggtatgc

SEQ ID NO: 123 - DNA, 16 bp, Artificial Sequence
  ASO against PSMD9
ctatatcccagacacc

SEQ ID NO: 124 - DNA, 16 bp, Artificial Sequence
  ASO against PSMD9
gatatatttgcaacaa

SEQ ID NO: 125 - DNA, 16 bp, Artificial Sequence
  ASO against PSMD9
cacttatctgttagct

SEQ ID NO: 126 - DNA, 16 bp, Artificial Sequence
  ASO against PSMD9
taatatgggagccttc

SEQ ID NO: 127 - DNA, 16 bp, Artificial Sequence
  ASO against PSMD9
tgctttagggccagct

SEQ ID NO: 128 - DNA, 16 bp, Artificial Sequence
  ASO against PSMD9
ataggatgtagctcgg

SEQ ID NO: 129 - DNA, 16 bp, Artificial Sequence
  ASO against PSMD9
tgatgtctttagcaca

SEQ ID NO: 130 - DNA, 16 bp, Artificial Sequence
  ASO against PSMD9
ataataaagccatcga

SEQ ID NO: 131 - DNA, 16 bp, Artificial Sequence
  ASO against PSMD9
accaatggcacactca

SEQ ID NO: 132 - DNA, 16 bp, Artificial Sequence
  ASO against PSMD9
tgattatctacagtgc

SEQ ID NO: 133 - DNA, 16 bp, Artificial Sequence
  ASO against PSMD9
ggcttacagtagagtc

SEQ ID NO: 134 - DNA, 16 bp, Artificial Sequence
  ASO against PSMD9
ataatattgaatcagg

SEQ ID NO: 135 - DNA, 16 bp, Artificial Sequence
  ASO against PSMD9
tgcaactatgccctga

SEQ ID NO: 136 - DNA, 16 bp, Artificial Sequence
  ASO against PSMD9
gctagcgcgggacaca

SEQ ID NO: 137 - DNA, 16 bp, Artificial Sequence
  ASO against PSMD9
aaaattactggtgctc

SEQ ID NO: 138 - DNA, 16 bp, Artificial Sequence
  ASO against PSMD9
gtcacacacggagagc

SEQ ID NO: 139 - DNA, 16 bp, Artificial Sequence
  ASO against PSMD9
ggagtaggcaggtgcc

SEQ ID NO: 140 - DNA, 16 bp, Artificial Sequence
  ASO against PSMD9
gacagatacccagcgc

SEQ ID NO: 141 - DNA, 16 bp, Artificial Sequence
  ASO against PSMD9
acctatatccacgggc

SEQ ID NO: 142 - DNA, 16 bp, Artificial Sequence
  ASO against PSMD9
tgagatgcgaccccct

SEQ ID NO: 143 - DNA, 16 bp, Artificial Sequence
  ASO against PSMD9
caagattgcttgcgct

SEQ ID NO: 144 - DNA, 16 bp, Artificial Sequence
  ASO against PSMD9
ttcttactgagacaca

SEQ ID NO: 145 - DNA, 16 bp, Artificial Sequence
  ASO against PSMD9
tccttaagttccggca

SEQ ID NO: 146 - DNA, 16 bp, Artificial Sequence
  ASO against PSMD9
tgggtaccagcatccc

SEQ ID NO: 147 - DNA, 16 bp, Artificial Sequence
  ASO against PSMD9
actctctatgggtacc

SEQ ID NO: 148 - DNA, 16 bp, Artificial Sequence
  ASO against PSMD9
ctatctgagcacactc

SEQ ID NO: 149 - DNA, 16 bp, Artificial Sequence
  ASO against PSMD9
agctctatctgagcac

SEQ ID NO: 150 - DNA, 16 bp, Artificial Sequence
  ASO against PSMD9
tgggtaccagcatcct

SEQ ID NO: 151 - DNA, 16 bp, Artificial Sequence
  ASO against PSMD9
tatgggtgccagcatc

SEQ ID NO: 152 - DNA, 16 bp, Artificial Sequence
  ASO against PSMD9
tctctatgggtgccag

SEQ ID NO: 153 - DNA, 16 bp, Artificial Sequence
  ASO against PSMD9
acactctctatgggtg

SEQ ID NO: 154 - DNA, 16 bp, Artificial Sequence
  ASO against PSMD9
tcccagctctatctga

SEQ ID NO: 155 - DNA, 16 bp, Artificial Sequence
  ASO against PSMD9
ggtaccagcattccca

SEQ ID NO: 156 - DNA, 16 bp, Artificial Sequence
  ASO against PSMD9
tatgggtaccagcatt

SEQ ID NO: 157 - DNA, 16 bp, Artificial Sequence
  ASO against PSMD9
agggattgagaagtga

SEQ ID NO: 158 - DNA, 16 bp, Artificial Sequence
  ASO against PSMD9
agccaacgcgcaagta

SEQ ID NO: 159 - DNA, 16 bp, Artificial Sequence
  ASO against PSMD9
ggctgaggctacgggt

SEQ ID NO: 160 - DNA, 16 bp, Artificial Sequence
  ASO against PSMD9
cccgacatcgcggacc

SEQ ID NO: 161 - DNA, 16 bp, Artificial Sequence
  ASO against PSMD9
cgcacgggcctcggag

SEQ ID NO: 162 - DNA, 16 bp, Artificial Sequence
  ASO against PSMD9
tcgcatcagatcctgg

SEQ ID NO: 163 - DNA, 16 bp, Artificial Sequence
  ASO against PSMD9
ggtacaagtccacatc

SEQ ID NO: 164 - DNA, 16 bp, Artificial Sequence
  ASO against PSMD9
cccgagcctgcttctc

SEQ ID NO: 165 - DNA, 16 bp, Artificial Sequence
  ASO against PSMD9
tgatactgttcactct

SEQ ID NO: 166 - DNA, 16 bp, Artificial Sequence
  ASO against PSMD9
ccgcaatactggctgg

SEQ ID NO: 167 - DNA, 16 bp, Artificial Sequence
  ASO against PSMD9
gttcacggagccgaac

SEQ ID NO: 168 - DNA, 16 bp, Artificial Sequence
  ASO against PSMD9
caccgtcacattcagg

SEQ ID NO: 169 - DNA, 16 bp, Artificial Sequence
  ASO against PSMD9
gcccagcgggttggaa

SEQ ID NO: 170 - DNA, 16 bp, Artificial Sequence
  ASO against PSMD9
agagaacgaggaaacg

SEQ ID NO: 171 - DNA, 16 bp, Artificial Sequence
  ASO against PSMD9
ccttacacttaaggga

SEQ ID NO: 172 - DNA, 16 bp, Artificial Sequence
  ASO against PSMD9
attacatcaggcagcc

SEQ ID NO: 173 - DNA, 16 bp, Artificial Sequence
  ASO against PSMD9
ttaataatgcctcaac

SEQ ID NO: 174 - DNA, 16 bp, Artificial Sequence
  ASO against PSMD9
taatttgcacaagacg

SEQ ID NO: 175 - DNA, 16 bp, Artificial Sequence
  ASO against PSMD9
ggctatggcctaattt

SEQ ID NO: 176 - DNA, 16 bp, Artificial Sequence
  ASO against PSMD9
tcaaatcattactcgc

SEQ ID NO: 177 - DNA, 16 bp, Artificial Sequence
  ASO against PSMD9
cacactgagtccgtct

SEQ ID NO: 178 - DNA, 16 bp, Artificial Sequence
  ASO against PSMD9
ccaatactaatgaggc

SEQ ID NO: 179 - DNA, 16 bp, Artificial Sequence
  ASO against PSMD9
tcagtatatgcctctc

SEQ ID NO: 180 - DNA, 16 bp, Artificial Sequence
  ASO against PSMD9
gcatgtacgaaattct

SEQ ID NO: 181 - DNA, 16 bp, Artificial Sequence
  ASO against PSMD9
agggacttgagatgac

SEQ ID NO: 182 - DNA, 16 bp, Artificial Sequence
  ASO against PSMD9
ggcacttagtaggtta

SEQ ID NO: 183 - DNA, 16 bp, Artificial Sequence
  ASO against PSMD9
atgaatatactgacag

SEQ ID NO: 184 - DNA, 16 bp, Artificial Sequence
  ASO against PSMD9
ctccagtggaagatcc

SEQ ID NO: 185 - DNA, 16 bp, Artificial Sequence
  ASO against PSMD9
ggtgatactgcagttg

SEQ ID NO: 186 - DNA, 16 bp, Artificial Sequence
  ASO against PSMD9
tgcgggtacactgagc

SEQ ID NO: 187 - DNA, 16 bp, Artificial Sequence
  ASO against PSMD9
caagatcccaagggta

SEQ ID NO: 188 - DNA, 16 bp, Artificial Sequence
  ASO against PSMD9
ctgtaacaaggtttgg

SEQ ID NO: 189 - DNA, 16 bp, Artificial Sequence
  ASO against PSMD9
tgcttcgcaggacttc

SEQ ID NO: 190 - DNA, 16 bp, Artificial Sequence
  ASO against PSMD9
acggagggacacttgc

SEQ ID NO: 191 - DNA, 16 bp, Artificial Sequence
  ASO against PSMD9
tcagaggatgcatcag

SEQ ID NO: 192 - DNA, 16 bp, Artificial Sequence
  ASO against PSMD9
atcatactgggtaacc

SEQ ID NO: 193 - DNA, 16 bp, Artificial Sequence
  ASO against PSMD9
gaggaagccaatatca

SEQ ID NO: 194 - DNA, 16 bp, Artificial Sequence
  ASO against PSMD9
aatcaggcccatctgc

SEQ ID NO: 195 - DNA, 16 bp, Artificial Sequence
  ASO against PSMD9
gcaagaataaccctca

SEQ ID NO: 196 - DNA, 16 bp, Artificial Sequence
  ASO against PSMD9
agctttaccaagccgg

SEQ ID NO: 197 - DNA, 16 bp, Artificial Sequence
  ASO against PSMD9
ggtttctaataggttt

SEQ ID NO: 198 - DNA, 16 bp, Artificial Sequence
  ASO against PSMD9
gttaccacgcatgtgt

SEQ ID NO: 199 - DNA, 16 bp, Artificial Sequence
  ASO against PSMD9
agcatttccgggctgg

SEQ ID NO: 200 - DNA, 16 bp, Artificial Sequence
  ASO against PSMD9
actgtatgggttgact

SEQ ID NO: 201 - DNA, 16 bp, Artificial Sequence
  ASO against PSMD9
ctttatacttagcctc

SEQ ID NO: 202 - DNA, 16 bp, Artificial Sequence
  ASO against PSMD9
ccatatgcactcctca

SEQ ID NO: 203 - DNA, 16 bp, Artificial Sequence
  ASO against PSMD9
gtgatgtctttagcac

SEQ ID NO: 204 - DNA, 16 bp, Artificial Sequence
  ASO against PSMD9
tacttttgtatgcagc

SEQ ID NO: 205 - DNA, 16 bp, Artificial Sequence
  ASO against PSMD9
tctaacaggtacttca

SEQ ID NO: 206 - DNA, 16 bp, Artificial Sequence
  ASO against PSMD9
tcttactctgcaccct

SEQ ID NO: 207 - DNA, 16 bp, Artificial Sequence
  ASO against PSMD9
ggtcatctagcctgcc

SEQ ID NO: 208 - DNA, 16 bp, Artificial Sequence
  ASO against PSMD9
cctactactgggctct

SEQ ID NO: 209 - DNA, 16 bp, Artificial Sequence
  ASO against PSMD9
aatataatcacatcgg

SEQ ID NO: 210 - DNA, 16 bp, Artificial Sequence
  ASO against PSMD9
ggatttgggagagcca

SEQ ID NO: 211 - DNA, 16 bp, Artificial Sequence
  ASO against PSMD9
cttcatctgtgacccg

SEQ ID NO: 212 - DNA, 16 bp, Artificial Sequence
  ASO against PSMD9
tccgaattcagaatcc

SEQ ID NO: 213 - DNA, 16 bp, Artificial Sequence
  ASO against PSMD9
ggtcatttgtaccgct

SEQ ID NO: 214 - DNA, 16 bp, Artificial Sequence
  ASO against PSMD9
gtgtaaaagactcagc

SEQ ID NO: 215 - DNA, 16 bp, Artificial Sequence
  ASO against PSMD9
ctactatccatttggg

SEQ ID NO: 216 - DNA, 16 bp, Artificial Sequence
  ASO against PSMD9
tgagggaccgctaaca

SEQ ID NO: 217 - DNA, 16 bp, Artificial Sequence
  ASO against PSMD9
cagaaattgttgttgc

SEQ ID NO: 218 - DNA, 16 bp, Artificial Sequence
  ASO against PSMD9
cttactccgagggtca

SEQ ID NO: 219 - DNA, 16 bp, Artificial Sequence
  ASO against PSMD9
tctatctgagcgcact

SEQ ID NO: 220 - DNA, 16 bp, Artificial Sequence
  ASO against PSMD9
cccagctctatctgag

SEQ ID NO: 221 - DNA, 16 bp, Artificial Sequence
  ASO against PSMD9
atgggtaccagcatcc

SEQ ID NO: 222 - DNA, 16 bp, Artificial Sequence
  ASO against PSMD9
cactctctatgggtac

SEQ ID NO: 223 - DNA, 16 bp, Artificial Sequence
  ASO against PSMD9
agcacactctctatgg

SEQ ID NO: 224 - DNA, 16 bp, Artificial Sequence
  ASO against PSMD9
tctatctgagcacact

SEQ ID NO: 225 - DNA, 16 bp, Artificial Sequence
  ASO against PSMD9
gggtgccagcatcccc

SEQ ID NO: 226 - DNA, 16 bp, Artificial Sequence
  ASO against PSMD9
ctctctatgggtgcca

SEQ ID NO: 227 - DNA, 16 bp, Artificial Sequence
  ASO against PSMD9
ttcccagctctatctg

SEQ ID NO: 228 - DNA, 16 bp, Artificial Sequence
  ASO against PSMD9
gggtaccagcattccc

SEQ ID NO: 229 - DNA, 16 bp, Artificial Sequence
  ASO against PSMD9
ttctatctgagcacac

SEQ ID NO: 230 - DNA, 16 bp, Artificial Sequence
  ASO against PSMD9
tgtatataagagagtc

SEQ ID NO: 231 - DNA, 16 bp, Artificial Sequence
  ASO against PSMD9
gctcagccaacgcgca

SEQ ID NO: 232 - DNA, 16 bp, Artificial Sequence
  ASO against PSMD9
gggtttcccggctacg

SEQ ID NO: 233 - DNA, 16 bp, Artificial Sequence
  ASO against PSMD9
cttcacccgacatcgc

SEQ ID NO: 234 - DNA, 16 bp, Artificial Sequence
  ASO against PSMD9
ggccgcacgggcctcg

SEQ ID NO: 235 - DNA, 16 bp, Artificial Sequence
  ASO against PSMD9
aattagccttgatctc

SEQ ID NO: 236 - DNA, 16 bp, Artificial Sequence
  ASO against PSMD9
tcggacctggtacaag

SEQ ID NO: 237 - DNA, 16 bp, Artificial Sequence
  ASO against PSMD9
cttcagccatgtcccg

SEQ ID NO: 238 - DNA, 16 bp, Artificial Sequence
  ASO against PSMD9
ctgatactgttcactc

SEQ ID NO: 239 - DNA, 16 bp, Artificial Sequence
  ASO against PSMD9
cgtcatccacttgcag

SEQ ID NO: 240 - DNA, 16 bp, Artificial Sequence
  ASO against PSMD9
ggtgttcacggagccg

SEQ ID NO: 241 - DNA, 16 bp, Artificial Sequence
  ASO against PSMD9
ctgcggatcaccgtca

SEQ ID NO: 242 - DNA, 16 bp, Artificial Sequence
  ASO against PSMD9
gcctagagaacgagga

SEQ ID NO: 243 - DNA, 16 bp, Artificial Sequence
  ASO against PSMD9
tccttacacttaaggg

SEQ ID NO: 244 - DNA, 16 bp, Artificial Sequence
  ASO against PSMD9
gattacatcaggcagc

SEQ ID NO: 245 - DNA, 16 bp, Artificial Sequence
  ASO against PSMD9
ctaatttgcacaagac

SEQ ID NO: 246 - DNA, 16 bp, Artificial Sequence
  ASO against PSMD9
agacaggctatggcct

SEQ ID NO: 247 - DNA, 16 bp, Artificial Sequence
  ASO against PSMD9
cgccagagtcatcccc

SEQ ID NO: 248 - DNA, 16 bp, Artificial Sequence
  ASO against PSMD9
atcaaatcattactcg

SEQ ID NO: 249 - DNA, 16 bp, Artificial Sequence
  ASO against PSMD9
ttacacactgagtccg

SEQ ID NO: 250 - DNA, 16 bp, Artificial Sequence
  ASO against PSMD9
gtatatgcctctcatc

SEQ ID NO: 251 - DNA, 16 bp, Artificial Sequence
  ASO against PSMD9
aatacatattcctcag

SEQ ID NO: 252 - DNA, 16 bp, Artificial Sequence
  ASO against PSMD9
tgcatgtacgaaattc

SEQ ID NO: 253 - DNA, 16 bp, Artificial Sequence
  ASO against PSMD9
ggaagtgggtacgagg

SEQ ID NO: 254 - DNA, 16 bp, Artificial Sequence
  ASO against PSMD9
acaaatggcacttagt

SEQ ID NO: 255 - DNA, 16 bp, Artificial Sequence
  ASO against PSMD9
caccatgaatatactg

SEQ ID NO: 256 - DNA, 16 bp, Artificial Sequence
  ASO against PSMD9
tggaaggttgaccaca

SEQ ID NO: 257 - DNA, 16 bp, Artificial Sequence
  ASO against PSMD9
gggtgatactgcagtt

SEQ ID NO: 258 - DNA, 16 bp, Artificial Sequence
  ASO against PSMD9
ggccagtgcgggtaca

SEQ ID NO: 259 - DNA, 16 bp, Artificial Sequence
  ASO against PSMD9
actataataccaggag

SEQ ID NO: 260 - DNA, 16 bp, Artificial Sequence
  ASO against PSMD9
ctaacctgtaacaagg

SEQ ID NO: 261 - DNA, 16 bp, Artificial Sequence
  ASO against PSMD9
ccggactgcggcccag

SEQ ID NO: 262 - DNA, 16 bp, Artificial Sequence
  ASO against PSMD9
gcccacggagggacac

SEQ ID NO: 263 - DNA, 16 bp, Artificial Sequence
  ASO against PSMD9
ggctacggtgactcca

SEQ ID NO: 264 - DNA, 16 bp, Artificial Sequence
  ASO against PSMD9
tatcatactgggtaac

SEQ ID NO: 265 - DNA, 16 bp, Artificial Sequence
  ASO against PSMD9
ttccagtgggttactg

SEQ ID NO: 266 - DNA, 16 bp, Artificial Sequence
  ASO against PSMD9
gcttaatctggctcca

SEQ ID NO: 267 - DNA, 16 bp, Artificial Sequence
  ASO against PSMD9
gttttaagctttacca

SEQ ID NO: 268 - DNA, 16 bp, Artificial Sequence
  ASO against PSMD9
ggcctttaagagttcc

SEQ ID NO: 269 - DNA, 16 bp, Artificial Sequence
  ASO against PSMD9
cacaattccacgctac

SEQ ID NO: 270 - DNA, 16 bp, Artificial Sequence
  ASO against PSMD9
agtactgggagatagc

SEQ ID NO: 271 - DNA, 16 bp, Artificial Sequence
  ASO against PSMD9
accaagattcctccca

SEQ ID NO: 272 - DNA, 16 bp, Artificial Sequence
  ASO against PSMD9
tccatatgcactcctc

SEQ ID NO: 273 - DNA, 16 bp, Artificial Sequence
  ASO against PSMD9
agtgatgtctttagca

SEQ ID NO: 274 - DNA, 16 bp, Artificial Sequence
  ASO against PSMD9
ctatttgcacagtggg

SEQ ID NO: 275 - DNA, 16 bp, Artificial Sequence
  ASO against PSMD9
caaaggatacaccacc

SEQ ID NO: 276 - DNA, 16 bp, Artificial Sequence
  ASO against PSMD9
tggtacagtaagctct

SEQ ID NO: 277 - DNA, 16 bp, Artificial Sequence
  ASO against PSMD9
ccttattcaacccagg

SEQ ID NO: 278 - DNA, 16 bp, Artificial Sequence
  ASO against PSMD9
tgtttagggttagcct

SEQ ID NO: 279 - DNA, 16 bp, Artificial Sequence
  ASO against PSMD9
tgcttattaggtgcta

SEQ ID NO: 280 - DNA, 16 bp, Artificial Sequence
  ASO against PSMD9
accataggtctctccc

SEQ ID NO: 281 - DNA, 16 bp, Artificial Sequence
  ASO against PSMD9
cgtataatagccccaa

SEQ ID NO: 282 - DNA, 16 bp, Artificial Sequence
  ASO against PSMD9
ttgtatgtcagttgcc

SEQ ID NO: 283 - DNA, 16 bp, Artificial Sequence
  ASO against PSMD9
ccgacttaccccctcg

SEQ ID NO: 284 - DNA, 16 bp, Artificial Sequence
  ASO against PSMD9
cccaataacagctgca

SEQ ID NO: 285 - DNA, 16 bp, Artificial Sequence
  ASO against PSMD9
ctctatagcaaggtgt

SEQ ID NO: 286 - DNA, 16 bp, Artificial Sequence
  ASO against PSMD9
cgtggcagcgcactgt

SEQ ID NO: 287 - DNA, 16 bp, Artificial Sequence
  ASO against PSMD9
tcaatactcatgttgt

SEQ ID NO: 288 - DNA, 16 bp, Artificial Sequence
  ASO against PSMD9
ccaatcaacaatctgg

SEQ ID NO: 289 - DNA, 16 bp, Artificial Sequence
  ASO against PSMD9
agctctatctgagcgc

SEQ ID NO: 290 - DNA, 16 bp, Artificial Sequence
  ASO against PSMD9
ggtaccagcatcccca

SEQ ID NO: 291 - DNA, 16 bp, Artificial Sequence
  ASO against PSMD9
tatgggtaccagcatc

SEQ ID NO: 292 - DNA, 16 bp, Artificial Sequence
  ASO against PSMD9
acactctctatgggta

SEQ ID NO: 293 - DNA, 16 bp, Artificial Sequence
  ASO against PSMD9
gagcacactctctatg

SEQ ID NO: 294 - DNA, 16 bp, Artificial Sequence
  ASO against PSMD9
ctctatctgagcacac

SEQ ID NO: 295 - DNA, 16 bp, Artificial Sequence
  ASO against PSMD9
tcagctctatctgagc

SEQ ID NO: 296 - DNA, 16 bp, Artificial Sequence
  ASO against PSMD9
tgggtgccagcatccc

SEQ ID NO: 297 - DNA, 16 bp, Artificial Sequence
  ASO against PSMD9
tctatgggtgccagca

SEQ ID NO: 298 - DNA, 16 bp, Artificial Sequence
  ASO against PSMD9
actctctatgggtgcc

SEQ ID NO: 299 - DNA, 16 bp, Artificial Sequence
  ASO against PSMD9
gggtgccagcatcctc

SEQ ID NO: 300 - DNA, 16 bp, Artificial Sequence
  ASO against PSMD9
cattcccagctctatc

SEQ ID NO: 301 - DNA, 16 bp, Artificial Sequence
  ASO against PSMD9
tgggtaccagcattcc

SEQ ID NO: 302 - DNA, 16 bp, Artificial Sequence
  ASO against PSMD9
gttctatctgagcaca

SEQ ID NO: 303 - DNA, 16 bp, Artificial Sequence
  ASO against PSMD9
gtgaacacttcttctc

(19) United States
(12) Patent Application Publication
(10) Pub. No.: US 2022/0282257 A1
(43) Pub. Date: Sep. 8, 2022

(54) METHOD OF MODULATING ADIPOSITY

(71) Applicants: BAKER HEART AND DIABETES INSTITUTE, Melbourne (AU); THE REGENTS OF THE UNIVERSITY OF CALIFORNIA, Oakland, CA (US)

(72) Inventors: Anna Christine DREW, Cheltenham (AU); Brian Gary DREW, Cheltenham (AU); Thomas de Aguiar VALLIM, Los Angeles, CA (US)

(21) Appl. No.: 17/636,481

(22) PCT Filed: Aug. 19, 2020
(86) PCT No.: PCT/AU2020/050860

§ 371 Date: Feb. 18, 2022

Related U.S. Application Data
(60) Provisional application No. 62/888,914, filed on Aug. 19, 2019.

(51) Int. Cl. C12N 15/113; A61P 3/04

(52) U.S. Cl. CPC C12N 15/1137; A61P 3/04; C12N 2310/11; C12N 2320/30; C12N 2310/341

(57) ABSTRACT

A method of modulating adiposity using PSMD9 inhibitors

### FIELD

The present application relates generally to energy homeostasis in adipose tissue, modulatory agents, screening methods and methods of using modulatory agents to modulate adipocyte energy homeostasis, reduce adipose weight gain or promote adipose weight loss.

### DESCRIPTION OF THE ART

The reference in this specification to any prior publication, or to any matter which is known, is not, and should not be taken as an acknowledgment or admission or any form of suggestion that that prior publication or known matter forms part of the common general knowledge in the field of endeavour to which this specification relates.

Bibliographic details of documents referred to are listed at the end of the specification. Sequences provided in the accompanying sequence listing are described in Table 7 at the end of the specification.

Body weight disorders such as overweight and obesity occur where there is abnormal or excessive lipid accumulation that may impair health. Studies of the genetics of human obesity, and of animal models of obesity, demonstrate that obesity results from complex defective regulation of both food intake, food induced energy expenditure, and of the balance between lipid and lean body anabolism. Obesity may be due to genetic and/or environmental factors. A crude population measure of obesity is the body mass index (BMI), a person's weight (in kilograms) divided by the square of their height (in metres). A person with a BMI of about 30 kg/m² or more is generally considered obese. A person with a BMI of more than about 25 kg/m² is considered overweight, although this figure may vary. Overweight and obesity are major risk factors for a number of chronic diseases, including diabetes, cardiovascular diseases and cancer. Once considered a problem only in high income countries, overweight and obesity are now dramatically on the rise in low- and middle-income countries, particularly in urban settings. The World Health Organisation published information concerning the scale of the problem in 2018, stating that in 2018 more than 1.9 billion adults 18 years and older were overweight and of these, 650 million were obese. The same year, 41 million children under the age of 5 were overweight or obese, and 340 million children and adolescents aged 5 to 19 were overweight or obese.

Adipose tissue, body fat, or simply fat is a loose connective tissue composed mostly of adipocytes which are specialised lipid storage cells. There are different types of adipose tissue generally referred to as white adipose tissue (WAT) brown adipose tissue (BAT) and beige adipose tissue (BEAT). In addition to adipocytes, adipose tissue contains the stromal vascular fraction (SVF) of cells including pre-adipocytes, fibroblasts, vascular endothelial cells and a variety of immune cells such as adipose tissue macrophages. Adipose tissue and adipocytes are the body's primary lipid storage vehicle however obesity and overweight are associated with lipid accumulation in non-adipose tissue where it interferes with healthy tissue processes and leads to disease.

Several well-established obesity treatment modes ranging from non-pharmaceutical to pharmaceutical intervention are known. Non-pharmaceutical interventions include diet, exercise, psychiatric treatment, and surgical treatments to reduce food consumption or remove fat.

There is a clear on-going need for effective interventions for obesity and overweight, collectively referred to as adiposity.

### SUMMARY OF THE DISCLOSURE

There is provided a method of reducing adiposity or increasing energy expenditure in adipose tissue in a mammalian subject in need thereof.

Reference to “adiposity” encompasses obesity and overweight and refers herein to storage of fat in adipose tissue, such as and including white adipose tissue. Adipose tissue and adiposity may be selected from white adipose tissue, brown adipose tissue, beige adipose tissue, and from types of fat depot locations selected from, for example, visceral fat (e.g., mesenteric, perirenal, epididymal, epicardial), subcutaneous fat, intramuscular fat, cervical adipose tissue, etc.

In one embodiment, reduced or reducing adiposity is a reduction in adipose tissue mass such as by promoting adipose weight loss.

In one embodiment, reduced or reducing adiposity is a reduction in gain of adipose tissue mass, such as by inhibiting or reducing adipose weight gain.

A “inhibition”, “reduction”, “reduced”, or “reducing” and the like refers to a level or percentage or relative to a level or range of levels or percentages in a control, which can be a control population or an earlier or later data point or range of data points for an individual subject. The level or range of levels may be direct or indirect measures of adipose homeostasis, adiposity, lipid levels (DA, TG, FFA), fatty acid oxidation, weight loss, reduced weight gain, lipolysis, adipose weight loss, adipose energy expenditure etc indicative of an enhanced propensity to either not gain adipose tissue in the presence of excess calories or to lose excess adipose tissue. In one embodiment, a reduction may be at least a 2% to 50% reduction or at least a 1% to 100% reduction. For example, FIG. 10 shows PSMD9 reduction in adipose tissue produced a showed a 28% reduction in weight gain compared on a high fat diet and a 38% reduction in fat mass. These reductions were not seen when the PSMD9 inhibitor ASO was targeted to the liver. For example, there was a more than 50% reduction in the expression levels of genes associated with lipid synthesis and lipid storage in WAT.

An “increase” “increasing” “elevated” and the like refers to a level or percentage or relative to a level or range of levels or percentages in a control, which can be a control population or an earlier or later data point or range of data points for an individual subject. The level or range of levels may be direct or indirect measures of adipose homeostasis, metabolism, weight loss, adiposity, fatty acid oxidation, lipolysis, adipose energy expenditure etc indicative of an enhanced propensity to either not gain adipose tissue in the presence of excess calories or to lose excess adipose tissue. In one embodiment increased energy expenditure, lipolysis, fatty acid oxidation, rate of adipose weight loss, percent body weight gain, may be at least a 2% to 50%, or at least a 1% to 100% increase. For example, there was a 2 to 50 fold increase in the expression of enzymes associated with WAT browning and increased metabolic activity as described further in the Examples.

Parker et al, Nature 567(7747):187-193, 2019 disclose the role of PSMD9 in regulating hepatic and plasma lipid abundance in a strain dependent manner at least in part via reductions in hepatic de novo lipogenesis. PSMD9 silencing with ASO was not associated with changes in body weight or food consumption. The present application describes the unexpected use of PSMD9 inhibition to reduce adiposity or prevent gain in adiposity in a mammalian subject and to increase energy expenditure. This was unexpected because it was previously contemplated that pathogenic lipids depleted from the liver by PSMD9 inhibition would be mobilised to the adipose tissue. Thus the present invention relating to adiposity does not relate to any reduction in ectopic fat found associated with the liver, or plasma lipid levels. As determined herein, administration of PSMD9 inhibitors reduces one or more key genes involved with lipogenesis and storage in adipose tissue and increases the expression of one or more genes pivotal in lipolysis and lipid metabolism within adipose tissue. In one embodiment, administration of PSMD9 inhibitors modulates adipocyte energy homeostasis by increasing fatty acid oxidation and lipolysis in adipose tissue permitting the use of PSMD9 inhibitors to modulate adiposity, such as by reducing excess adipose weight gain and promote excess adipose weight loss.

Adiposity may be assessed directly or indirectly. The effect of reducing adiposity can be measured directly by, for example monitoring changes in size (e.g. waist circumference for central adiposity), body weight or body fat distribution or percentage (e.g. DEXA scan), fat mass (EcoMRI) or indirectly by any method such as monitoring changes in levels of lipids/fatty acids, acylglycerols, markers of lipolysis or fatty acid oxidation, glyceride hydrolysis, lipogenesis, lipid metabolism, energy expended, or expression of genes/polypeptides associated or correlated therewith or adiposity, in one or more subjects or populations, such as by (but not in any way limited to) the methods described herein.

Accordingly, in one embodiment the present application enables a method of reducing adiposity, reducing adipose weight gain or promoting adipose weight loss in a mammalian subject, comprising administering a PSMD9 inhibitor to the subject. In another aspect, the present application provides a method of treating or preventing obesity in a mammalian subject, comprising administering a PSMD9 inhibitor to the subject. In one embodiment, the PSMD9 inhibitor comprises an agent that inhibits PSMD9 expression or PSMD9 polypeptide activity.

In one embodiment, reducing adiposity, reducing adipose weight gain or promoting adipose weight loss may be at least a 5%, 10%, 15%, 20%, 25%, 30%, 35% 40%, 45%, 50%, 55%, 60%, 65%, 70%, 65%, 70%, 75%, 80%, 85%, 90%, or 99% reduction in adiposity by weight, % adiposity, or reduction in weight gain relative to a control.

In one embodiment, the PSMD9 inhibitor is or comprises a peptide, a peptidomimetic, a small molecule, a polynucleotide, or a polypeptide. In one embodiment, the peptide is a phosphopeptide or phosphomimetic.

In one embodiment, the polypeptide comprises an anti-PSMD9 antibody or an antigen binding fragment thereof.

In one embodiment, the PSMD9 inhibitor is a polynucleotide. In one embodiment, the polynucleotide is a modified oligonucleotide targeting PSMD9. In one embodiment, the compound is single-stranded. In one embodiment, the compound is double-stranded.

In one embodiment, the modified oligonucleotide targeting PSMD9 comprises at least one modification selected from at least one modified internucleoside linkage, at least one modified sugar moiety, and at least one modified nucleobase.

In one embodiment, the modified oligonucleotide targeting PSMD9 comprises:
A gap segment consisting of linked deoxynucleotides;
A 5′ wing segment consisting of linked nucleosides;
A 3′ wing segment consisting of linked nucleosides;

wherein the gap segment is positioned immediately adjacent to and between the 5′ wing segment and the 3′ wing segment and wherein each nucleoside of each wing segment comprises a modified sugar.

In one embodiment, the PSMD9 inhibitor is an iRNA, such as an shRNA, siRNA, miRNA.

In one embodiment, the PSMD9 inhibitor is a polynucleotide which is a vector for the expression of the PSMD9 inhibitor.

In one embodiment, the PSMD9 inhibitor is a polynucleotide wherein the vector is a viral vector known in the art. Viral vectors useful for targeting specific tissues are known in the art.

In one embodiment, the PSMD9 inhibitor is administered in an amount and over a time effective to reduce adipose tissue weight gain or promoting adipose tissue weight loss in the subject.

In one embodiment, the PSMD9 inhibitor is administered in an amount effective to increase at least one measure of lipolysis, fatty acid oxidation, lipid metabolism or decrease lipogenesis in adipose tissue in the subject.

Illustrative effective amounts include a dose of 0.5 mg/kg to 70 mg/kg, such as 15 mg/kg, 20 mg/kg, 25 mg/kg, 30 mg/kg, 35 mg/kg and 50 mg/kg depending upon the mode of administration.

In one embodiment, the adipose tissue is visceral or epididymal adipose tissue.

In one embodiment, the adipose tissue is WAT.

In one embodiment, the method further comprises measuring weight loss or reduced weight gain in the subject in conjunction with PSMD9 inhibitor administration over a period of time. Suitable measures include total mass, fat mass or distribution, central size or volume etc.

In another aspect the present application provides a PSMD9 inhibitor as described herein for use in reducing adiposity in a subject in need thereof.

In one embodiment, the inhibitor increases lipolysis or triglyceride hydrolysis or lipid metabolism or metabolism, or decreases lipogenesis in adipose tissue in the subject.

In one embodiment, the PSMD9 inhibitor is or comprises a peptide, a peptidomimetic, a small molecule, a polynucleotide, or a polypeptide. In one embodiment, the peptide is a phosphopeptide or phosphomimetic.

In one embodiment, the polypeptide comprises an anti-PSMD9 antibody or an antigen binding fragment thereof.

In one embodiment, the PSMD9 inhibitor is a polynucleotide. In one embodiment, wherein the polynucleotide is a modified oligonucleotide targeting PSMD9. In one embodiment, wherein the compound is single-stranded. In one embodiment, wherein the compound is double-stranded.

In one embodiment, the modified oligonucleotide targeting PSMD9 comprises at least one modification selected from at least one modified internucleoside linkage, at least one modified sugar moiety, and at least one modified nucleobase.

In one embodiment, the modified oligonucleotide targeting PSMD9 comprises:
A gap segment consisting of linked deoxynucleotides;
A 5′ wing segment consisting of linked nucleosides;
A 3′ wing segment consisting of linked nucleosides;

wherein the gap segment is positioned immediately adjacent to and between the 5′ wing segment and the 3′ wing segment and wherein each nucleoside of each wing segment comprises a modified sugar.

In one embodiment, the PSMD9 inhibitor is an iRNA, such as an shRNA, siRNA, miRNA.

In one embodiment, the PSMD9 inhibitor is a polynucleotide which is a vector for the expression of the PSMD9 inhibitor.

In one embodiment, the PSMD9 inhibitor is a polynucleotide wherein the vector is a viral vector known in the art. Viral vectors useful for targeting specific tissues are known in the art.

In one embodiment, the PSMD9 inhibitor agent or a conjugate or vehicle comprising same comprises or is associated with an adipose homing peptide to facilitate delivery to adipose tissue.

In another aspect the present application enables and describes a pharmaceutical composition comprising a PSMD9 inhibitor and a pharmaceutically acceptable carrier and/or diluent for use in reducing adiposity in a subject.

In one embodiment, the inhibitor is effective to increase lipolysis or fatty acid oxidation or decrease lipogenesis in adipose tissue in the subject.

In one embodiment, energy expenditure increases or increased lipolysis, or lipid metabolism in adipose tissue by administration of PSMD9 inhibitors are at least 5%, 10%, 15%, 20%, or 25% relative to a control.

In another aspect the present application enables and describes the use of a PSMD9 inhibitor in the manufacture of a medicament for use in the treatment or prevention of adiposity.

Each embodiment described herein is to be applied mutatis mutandis to every other embodiment unless expressly stated otherwise.

### BRIEF DESCRIPTION OF THE FIGURES

The patent or application file contains at least one drawing executed in colour. Copies of this patent or patent application publication with colour drawing(s) will be provided by the Patent Office upon request and payment of the necessary fee.

The accompanying drawings, which are incorporated into and form a part of the specification, illustrate several embodiments of the present invention and, together with the description, serve to explain the principles of the invention.

FIG. 1. (a) cartoon of protocol of administration of ASO; (b) Western blot of PSMD9 and loading control (β-actin) from epididymal fat of C57BL/6J and DBA/2J mice after twice weekly (biweekly) intraperitoneal injection of ASO and feeding of a western diet for 4 weeks (c) graphical representation of (b). ****p<0.0001 vs control ASO

FIG. 2. (a) body weight gain of C57BL/6J and DBA/2J mice over duration of study where d in the x-axis denotes day from commencement of injection of 3 different ASOs against PSMD9 (ASO3, 5 & 6) and one control ASO (scramble) provided concomitantly with Western diet administration; (b) AST and ALT plasma levels at study end.

FIGS. 3 (a) and (b): Lipids Levels in Adipose Tissue following PSMD9 Knock Down in (a) C57BL/6J and (b) DBA/2J Mice—Lipidomic analysis (ESI-MS/MS) of epididymal adipose tissue. Data demonstrated changes in lipid classes relative to control ASO treated mice. (TG=triglyceride, DG=diglyceride, FFA=free fatty acid, COH=cholesterol, CE=cholesterol ester, Cer=ceramide, dhCer=dihydroceramide. *=p<0.05

FIGS. 4 (a) and (b): Fatty Acid (FA) Levels in Adipose Tissue following PSMD9 Knock Down in (a) C57BL/6J and (b) DBA/2J Mice—Lipidomic analysis (ESI-MS/MS) of epididymal adipose tissue. Data demonstrated changes in fatty acid (FA) levels relative to control ASO treated mice.

FIG. 5 (a) to (e): Alteration in Protein Levels in Adipose Tissue of PSMD9 Knock Down in C57BL/6J—Western blot of PSMD9 in epididymal fat of C57BL/6J mice after 4 weeks of IP injection of ASO and feeding of a western diet. The observed activation of AMPK (increased pAMPKa1) is consistent with increased energy expenditure.

FIG. 6 (a) to (e): Alteration in Protein Levels in Adipose Tissue of PSMD9 Knock Down in DBA/2J—Western blot of PSMD9 in epididymal fat of DBA/2J mice after 4 weeks of IP injection of ASO and feeding of a western diet. The observed activation of AMPK (increased pAMPKa1) is consistent with increased energy expenditure.

FIG. 7 (a) to (d): Adipose tissue mRNA expression following D9 ASO—mRNA expression in epididymal fat of C57BL/6J and DBA/2J mice after 4 weeks of IP injection of ASO and feeding of a western diet. A reduction in genes linked to lipogenesis and storage, and an increase in genes linked to fat oxidation were observed. Furthermore, changes in molecules involved in adipocyte energy homeostasis were also observed. Both C57BL/6J and DBA/2J mice were studied to determine whether the effect of PSMD9 knockdown would persist in the context of different genetic backgrounds (as seen in humans). Furthermore, these strains differ in lipid metabolism, with DBA/2J mice exhibiting increased basal lipid levels.

FIG. 8: provides an illustration of the Study Design and oligonucleotides for trials described in Examples 3 to 6.

FIG. 9: shows reduced PSMD9 mRNA expression in white adipose tissue (WAT) in epididymal (reduced by 93%) and subcutaneous (reduced by 85%) WAT at end of study described in Example 3 in Native PSND9 ASO compared to the native control. Liver targeted ASO displayed a reduced effect of epididymal (reduced by 54%) and subcutaneous (reduced by 28%) WAT, compared to a scrambled control.

FIG. 10A to E: illustrates data showing the native ASO causing significant reduction in weight gain (28% reduction compared to control) and fat mass (a 38% reduction) (10A, B, C) over the trial period described in Example 3 but not the liver-targeted ASO.

FIG. 11: illustrates data showing native ASO reduces adipose tissue weights. Organ weights subcutaneous fat mass (reduced by 61% with native ASO), brown adipose tissue (32% reduction with native ASO), liver mass (no significant difference with either native or liver targeted ASO), epididymal fat, epididymal fat mass (reduced by 63% by native ASO) at the end of the study described in Example 3.

FIG. 12: illustrates data showing a significant improvement in fasting blood glucose (FIGS. 12 A and B—19% reduction observed) and glucose handling (FIG. 12A, C—a 16% reduction was observed) with the native ASO. Mice underwent an oral glucose tolerance test (2 mg/kg lean mass); AUC—area under the curve.

FIG. 13: illustrates data showing no evidence of significant toxicity with regard to bilirubin levels or albumin in the blood for each treatment group (FIG. 13 B, C). A small increase (×4) in plasma ALT was observed with native ASO (FIG. 13A).

FIG. 14: illustrates data showing high significant and multifaceted molecular changes in adipose tissue during treatment with native PSMD9 ASO not seen with the liver targeted ASO. mRNA expression level were determined in epididymal white adipose tissue (WAT) showing significant reductions in the expression of genes associated with lipid synthesis: ACACB—acetyl co-A carboxylase beta (68% reduction), FASN—fatty acid synthase (89% reduction), and SCD-1—stearoyl-CoA desaturase-1 (88% reduction), and storage namely DGAT2—diacylglycerol o-acyltransferase 2 (90% reduction). There were also reductions in Angptl4-LPL axis which is involved in the hydrolysis of circulating lipids, reductions in the hydrolysis of lipid stores (CGI-58, HSL), a reduction in CEBP/α (down 47%), which has been shown drive the formation of new fat cells, as well as and a reduction in PPARα (down 70%), which drives the utilisation of lipids for energy.

FIG. 15: illustrates data showing changes in protein activity/levels in adipose tissue. Western blots of epididymal WAT showed reductions in ACC, reductions in protein expression of AMPKα, AMPKβ2 and an increase in AMPKα phosphorylation (pAMPK), which indicates an upregulation of catabolism. There was also trend for a reduction in phosphorylation of HSL (pHSL), demonstrating altered lipolysis activity. ACC—acetyl co-A carboxylase; AMPK—protein kinase AMP-activated catalytic subunit; HSL—hormone sensitive lipase.

FIG. 16: illustrates data showing mRNA expression in subcutaneous white adipose tissue (WAT). There were significant reductions in the expression of genes associated with lipid synthesis (FASN, SCD1) and storage (DGAT2). Also, reductions in LPL and a trend for a reduction in CGI-58, involved in the hydrolysis of circulating lipids and lipid stores respectively. ACACB—acetyl co-A carboxylase beta; FASN—fatty acid synthase; SCD-1—stearoyl-CoA desaturase-1; DGAT2—diacylglycerol o-acyltransferase 2; Angptl—angiopoietin-like; CGI-58 (ABHD5)—abhydrolase domain containing 5; HSL (LIPE)—hormone sensitive lipase; LPL—lipoprotein lipase; FIG. 16 (Coned)—mRNA expression in subcutaneous white adipose tissue (WAT) CEBP—CCAAT enhancer binding protein; PPAR—peroxisome proliferator activated receptor; Cox7a1—cytochrome C oxidase subunit; UCP1—uncoupling protein 1; Elov13—fatty acid elongation 3

### DETAILED DESCRIPTION OF PARTICULAR EMBODIMENTS

Before describing the present disclosure in detail, it is to be understood that unless otherwise indicated, the subject disclosure is not limited to specific formulations of components, manufacturing methods, dosage or diagnostic regimes, or the like. It is also to be understood that the terminology used herein is for the purpose of describing particular embodiments only and is not intended to be limiting.

The present specification enables and supports silencing or reduction of PSMD9 in adipose tissue to accrue beneficial changes to whole body metabolism that lead to improvements in obesity and its complications. The specification enables decreasing PSMD9 in adipose tissue to drive reductions in weight gain, suppression of lipid synthesis and storage within adipose tissue per se as well as enhanced energy expenditure.

Reductions in PSMD9 in adipose tissue led to reduced weight gain on a diet high in fat, activated pathways that promote the burning of energy, and reduced blood glucose levels. These specific findings indicate that reduction of PSMD9 in adipose tissue can be used to prevent or reduce weight gain and the accumulation of fat tissue in the setting of excess caloric intake or other causes of obesity such as, for example, sedentary behaviour or genetic predisposition, reduction of excess weight and fat mass in the setting of pre-existing obesity induced excess caloric intake, sedentary behaviour or genetic predisposition, deliver improvements in blood glucose levels in individuals who are obese and that have glucose intolerance, insulin resistance or type 2 diabetes, promote conversion of white adipose tissue from a storage unit for fat, to a tissue that burns fat for energy, activate cellular pathways in adipose tissue that liberate fat from intracellular stores (lipolysis) for the purposes of energy production, and reduce the risk of other complications associated with obesity such as glucose intolerance, and insulin resistance, and fatty liver disease and cardiovascular disease.

There is growing evidence of the important roles of key enzymes in and their link to fatty liver diseases in man. Indeed, DNL assessments such as by stable label techniques or fatty acid profiling is recognised as providing an instrumental marker of drug efficacy for novel NAFLD drugs and response to nutraceutical agents. In a review article by Tacer and Rozman J. lipids 2011 783976, the authors highlight the importance of genes in the DNL pathway in NAFLD and illustrate how their intervention is associated with reduction in NAFLD. Other relevant publications include: Jiang et al., J Clin Invest. 2005 April; 115(4):1030-8. Epub 2005 Mar. 10 showing inhibition of Stearoyl-CoA desaturase-1 (SCD1) reduced adiposity in mice. See also Xing Xian Yu et al. Hepatology; 42:362-371, 2005 who demonstrate antisense inhibition of acyl-coenzyme A:diacylglycerol acyltransferase 2 (DGAT2) reduces hepatic tryglyceride (TG) content and steatosis in mice; and Singh et al PloS One 2016 0164133 who showed that fatty acid synthase inhibitor, Platensimysin reduced DNL in lean and type 2 diabetes (T2D) monkeys and lowered plasma glucose.

As described in WO 2019/140488, administration of down modulators of PSMD9 is effective to prevent or treat the accumulation of pathological lipids in the subject. Specifically, down modulation of PSMD9 expression prevented or reduced pathological lipid accumulation at least in the liver and plasma of a subject. Inhibition of PSMD9 with antisense oligonucleotides caused a significant reduction in key enzymes in the DNL pathway (including ACACA, ACACAB, FASN, SCD) in mice on a high fat (Western) diet and a significant reduction in key pathological lipids linked to fatty liver disease including diacylglycerols (DGs) and triacylglycerol (TGs). Reference to “pathological lipids” includes one or more lipid species from a lipid class selected from acyl glycerols, diacylglycerol (DG) and triacylglycerol (TG), phosphatidylcholine (PC), phosphatidylethanolamine (PE), cholesteryl ester (CE) and ceramide (Cer) or their variants (e.g., dihexosylceramide (DHC)).

As described in WO 2019/140488 inhibition of PSMD9 in mice exposed to a Western diet for four weeks caused a reduction in markers of fibrosis (Vimentin, Smad7, collagen), ER stress (CHOP) and blood glucose. As described in WO 2019/140488 PSMD9 is a key regulator of the liver lipidome whose modulation permits favourable in vivo lipid remodelling (i.e., reduction in pathological lipid accumulation). Useful in the treatment or prevention of metabolic syndrome, fatty liver, fatty liver disease, NASH T2D or insulin resistance. However, a reduction in these enzymes affected the liver and did not cause a reduction in adiposity which was furthermore not expected. Accordingly, the present invention is surprising. Furthermore, the present invention provides treatment or prevention of obesity/adiposity and one or more of metabolic syndrome, fatty liver, fatty liver disease, NASH T2D or insulin resistance. As determined herein PSMD9 downregulation provides independent effects on liver and adipose tissue. For example, PSMD9 reduction/inhibition acts directly on liver tissue and adipose tissue to (i) improve hepatic lipid dysregulation and (ii) reduce adiposity increase metabolism in non-hepatic tissue.

Unless defined otherwise, all technical and scientific terms used herein have the same meaning as commonly understood by those of ordinary skill in the art to which the invention belongs.

As used herein the singular forms “a”, “an” and “the” include plural aspects unless the context clearly dictates otherwise. Thus, for example, reference to “a lipid species” includes a single lipid species, as well as two or more lipid species, reference to “the disclosure” includes single and multiple aspects of the disclosure and so forth.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

The term “and/or”, e.g., “X and/or Y” shall be understood to mean either “X and Y” or “X or Y” and shall be taken to provide explicit support for both meanings or for either meaning.

The term inhibitor or antagonist includes an agent that effects complete silencing of PSMD9 at the mRNA or protein level and an agent that indices partial silencing of PSMD9 activity in adipose tissue.

The terms “agent”, “antagonist”, “inhibitor, “modulator”, “compound”, “pharmacologically active agent”, “medicament” and “active” may be used interchangeably herein to refer to a substance or a combination of two or more substances that induces a desired pharmacological and/or physiological effect. The terms also encompass pharmaceutically acceptable and pharmacologically active forms thereof, including but not limited to salts, esters, amides, prodrugs, active metabolites, analogs and the like.

The terms “effective amount” and “therapeutically effective amount” and “prophylactically effective amount” as used herein mean a sufficient amount of an agent which provides the desired therapeutic or physiological effect or outcome, such as increased energy expenditure by adipose tissue, reducing excess adipose weight gain or promoting excess adipose weight loss, reducing TG, DG or FFA levels in adipose tissue, etc Undesirable effects, e.g. side effects, are sometimes manifested along with the desired effect; hence, a practitioner balances the potential benefits against the potential risks in determining what is an appropriate “effective amount”. The exact amount of agent required will vary from subject to subject, depending on the species, age and general condition of the subject, mode of administration and the like. Thus, it may not be possible to specify an exact “effective amount”.

A “subject” as used herein refers to an animal, preferably a mammal and more preferably a human who can benefit from the pharmaceutical compositions and methods of the present disclosure. There is no limitation on the type of animal that could benefit from the presently described pharmaceutical compositions and methods. A subject regardless of whether a human or non-human animal may be referred to as an individual, patient, animal, host or recipient as well as subject. The compounds and methods of the present disclosure have applications in human medicine and veterinary medicine. A subject in need as referred to herein is a subject who is overweight or obese or who is at risk of overweight or obesity, using recognized criteria. In some embodiments, the subject is not afflicted with one or more of fatty liver, NAFLD, NASH or T2D although clearly these are commonly associated with obesity. In one embodiment, the subject is not afflicted T2D or their T2D or insulin is under control or being treated with a different agent that does not inhibit the expression or activity of PSMD9.

A reduction in lipids, lipid species and pathological lipid species may be 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% relative to a suitable control. In some embodiments, reduction is 20%, 30%, 40% 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, 97%, 98%, or 99% or 100% or more, relative to a suitable control.

### Administration

Administration of PSMD9 inhibitors is generally in the form of a pharmaceutically or physiologically acceptable composition or an acceptable salt or stereoisomer thereof.

In one embodiment, the PSMD9 inhibitor is administered systemically. As used herein “systemic administration” is a route of administration that is either enteral or parenteral.

As used herein “enteral” refers to a form of administration that involves any part of the gastrointestinal tract and includes oral administration of, for example, the antisense oligonucleotide in tablet, capsule or drop form; gastric feeding tube, duodenal feeding tube, or gastrostomy; and rectal administration of, for example, the antisense compound in suppository or enema form.

As used herein “parenteral” includes administration by injection or infusion. Examples include, intravenous (into a vein), intraarterial (into an artery), intramuscular (into a muscle), intracardiac (into the heart), subcutaneous (under the skin), intraosseous infusion (into the bone marrow), intradermal, (into the skin itself), intrathecal (into the spinal canal), intraperitoneal (infusion or injection into the peritoneum), intravesical (infusion into the urinary bladder), intraperitoneal. Transdermal (diffusion through the intact skin), transmucosal (diffusion through a mucous membrane), inhalational.

In one embodiment, administration is subcutaneous.

In one embodiment, administration is in an amount effective to promote increased energy expenditure in adipose tissue. In one embodiment, the adipose tissue is WAT.

Administration may be as a single dose or as repeated doses on a period basis, for example, daily, weekly or monthly, once every two days, three, four, five, six seven, eight, nine, ten, eleven, twelve, thirteen or fourteen days, once weekly, twice weekly, three times weekly, or every two weeks, or every three weeks, or every four weeks, every two to 12 months. In one embodiment, the inter-dosing interval is 4 to 8 or 6 to 8 or 5 to 12 months. Inter-dosing interval may vary over the course of treatment as known to those of skill in the art.

In one embodiment, administration is 1 to 10 times per week, or once every week, two weeks, three weeks, four weeks, or once every two months.

Illustrative doses are between about 10 to 200 mg or between about 100 mg to 500 mg inclusive. Illustrative doses include 5, 10, 20, 25, 50, 100, 150, 200, 250, 300, 400, 500, 1000, 1500, 2000, 2500 mg. Illustrative doses include 1.5 mg/kg (about 50 to 100 mg) and 3 mg/kg (100-200 mg) and 4.5 mg/kg (150-300 mg). Further illustrative doses include 5 mg/kg, 10 mg/kg, 15 mg/kg, 20 mg/kg, 25 mg/kg (13 to 2500 mg), 30 mg/kg, 35 mg/kg or 50 mg/kg daily, weekly, monthly, bi-weekly or bi-monthly, six monthly or yearly.

The terms “therapeutically effective amount” or “prophylactically effective amount” are used herein to refer to a dose of the PSMD9 inhibitor sufficient for example to improve one or more markers, signs or symptoms of adiposity.

Brown fat or browning of white or beige fat can be assessed using art recognised measures for adaptive thermogenesis. BAT activity may, for example, be monitored by measuring the supraclavicular skin temperature, or by imaging such as nuclear imaging using positron emission tomography (PET) or PET and computer tomography (CT) imaging using for example, 18F-fluorodeoxyglucose (FDG). BAT activity may be enhanced over the course of treatment or by the end of treatment by at least 10%, 12%, 15%, 17%, 20% or 25%, 30%, or at least 35%, relative to the BAT activity, in a subject or study group prior to administration

In one embodiment, energy expenditure is enhanced. In one embodiment, energy expenditure is enhanced over the course of treatment sufficiently to induce significant weight/adipose tissue loss. In one embodiment, for example, energy expenditure and fat oxidation may be measured be indirect calorimetry in respiration chambers on a fixed activity protocol. In one embodiment, markers of body adipose content such as those derived from DEXA scanning are employed.

### Pharmaceutical Forms

The pharmaceutical forms suitable for injectable use include sterile aqueous solutions (where water-soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the agents in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

For parenteral administration, the agent may dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like.

For transmucosal or transdermal administration, penetrants appropriate to the barrier to be permeated can be used for delivering the agent. Such penetrants are generally known in the art e.g. for transmucosal administration, bile salts and fusidic acid derivatives. In addition, detergents can be used to facilitate permeation. Transmucosal administration can be through nasal sprays or using suppositories e.g. Sayani and Chien, Crit Rev Ther Drug Carrier Syst 13:85-184, 1996. For topical, transdermal administration, the agents are formulated into ointments, creams, salves, powders and gels. Transdermal delivery systems can also include patches.

For inhalation, the agents of the disclosure can be delivered using any system known in the art, including dry powder aerosols, liquids delivery systems, air jet nebulizers, propellant systems, and the like, see, e.g., Patton, Nat Biotech 16:141-143, 1998; product and inhalation delivery systems for polypeptide macromolecules by, e.g., Dura Pharmaceuticals (San Diego, Calif.), Aradigm Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, for example, air jet nebulizers. The PSMD9 reduction agent can also be administered in sustained delivery or sustained release mechanisms, which can deliver the formulation internally. For example, biodegradable microspheres or capsules or other biodegradable polymer configurations capable of sustained delivery of agonists can be included in the formulations of the disclosure (e.g. Putney and Burke, Nat Biotech 16:153-157, 1998).

In preparing pharmaceuticals of the present disclosure, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the disclosure in vesicles composed of substances such as proteins, lipids (for example, liposomes, see below), carbohydrates, or synthetic polymers (discussed above). For a general discussion of pharmacokinetics, see, e.g., Remington's.

In one aspect, the pharmaceutical formulations comprising agents of the present disclosure are incorporated in lipid monolayers or bilayers such as liposomes, see, e.g., U.S. Pat. Nos. 6,110,490; 6,096,716; 5,283,185 and 5,279,833. The disclosure also provides formulations in which water-soluble modulatory agents of the disclosure have been attached to the surface of the monolayer or bilayer. For example, peptides can be attached to hydrazide-PEG-(distearoylphosphatidyl)ethanolamine-containing liposomes (e.g. Zalipsky et al., Bioconjug Chem 6:705-708, 1995). Liposomes or any form of lipid membrane, such as planar lipid membranes or the cell membrane of an intact cell e.g. a red blood cell, can be used. Liposomal formulations can be by any means, including administration intravenously, transdermally (Vutla et al., J Pharm Sci 85:5-8, 1996), transmucosally, or orally. The disclosure also provides pharmaceutical preparations in which the nucleic acid, peptides and/or polypeptides of the disclosure are incorporated within micelles and/or liposomes (Suntres and Shek, J Pharm Pharmacol 46:23-28, 1994; Woodle et al., Pharm Res 9:260-265, 1992). Liposomes and liposomal formulations can be prepared according to standard methods and are also well known in the art see, e.g., Remington's; Akimaru et al., Cytokines Mol. Ther. 1:197-210, 1995; Alving et al., Immunol Rev 145:5-31, 1995; Szoka and Papahadjopoulos, Ann Rev Biophys Bioeng 9:467-508, 1980, U.S. Pat. Nos. 4,235,871, 4,501,728 and 4,837,028.

The pharmaceutical compositions of the disclosure can be administered in a variety of unit dosage forms depending upon the method of administration. Dosages for typical pharmaceutical compositions are well known to those of skill in the art. Such dosages are typically advisorial in nature and are adjusted depending on the particular therapeutic context, patient tolerance, etc. The amount of agent adequate to accomplish this is defined as the “effective amount”. The dosage schedule and effective amounts for this use, i.e., the “dosing regimen” will depend upon a variety of factors, including the stage of the fatty liver, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., “Remington's”.

“PSMD9” is annotated as proteosome 26S subunit, non-ATPase 9. SEQ ID NOs 1 (human) and 3, 10, 11 (mouse) provide human and mouse nucleotide sequence for human and mouse PSMD9. Other variant sequences are known in the art and all variants are expressly contemplated herein for use in the production of suitable modulators using art recognized methods. The term “identity” or “identical” as used herein refers to the percentage number of amino acids that are identical or constitute conservative substitutions. Identity may be determined using sequence comparison programs such as GAP (Deveraux et al., 1984, Nucleic Acids Research 12, 387-395), which is incorporated herein by reference. In this way sequences of a similar or substantially different length to those cited herein could be compared by insertion of gaps into the alignment, such gaps being determined, for example, by the comparison algorithm used by GAP. Reference to “PSMD9” herein includes mammalian isoforms, mutants, variants, and homologs or orthologs from various species, including without limitation murine and human forms. Mouse and human protein PSMD9 sequences are highly homologous as determined by NCBI BLAST based on illustrative full length sequences.

As used herein, a subject “at risk” of developing adiposity may or may not have obvious signs of overweight or obesity, and may or may not have displayed overweight or obesity prior to the treatment according to the present disclosure. “At risk” denotes that a subject has one or more risk factors, which are measurable parameters that correlate with a risk of development of adiposity, as known in the art and/or described herein.

As used herein, the terms “treating”, “treat” or “treatment” is an approach for obtaining beneficial physical or physiological or desired clinical results in at least some subjects. These include administering an inhibitor as described herein to thereby reduce fatty acid levels in adipose tissue in the subject to reduce or eliminate obesity or overweight in at least a proportion of subjects in need thereof.

As used herein, the terms “preventing”, “prevent” or “prevention” include administering a PSMD9 modulator to reduce body weight gain, or hinder the development of overweight or obesity in at least a proportion of subjects in need thereof. As determined herein a reduced weight gain may be a lower proportion of the body weight gain displayed by control subjects not given the modulator. For example, the % body weight gain may be, for example, 5% to 50% less than the weight gain by control subject not given the modulator.

An “effective amount” of a PSMD9 inhibitor refers to at least an amount effective, at dosages and for periods of time necessary, to achieve the desired result. In one embodiment, the effective amount provides a particular desirable % inhibition of PSMD9 in adipose tissue. In one embodiment, the effective amount provides a particular desirable % inhibition of PSMD9 in adipose tissue for a particular desirable time frame. For example, the desired result may be a therapeutic or prophylactic result. An effective amount can be provided in one or more administrations. In one embodiment, the term “effective amount” is meant an amount necessary to effect treatment of overweight or obesity. In some examples of the present disclosure, the term “effective amount” is meant an amount necessary to effect a change in a factor associated with overweight or obesity, such as over expression of PSMD9, levels of adipose tissue (such as mass or composition), lipolysis or lipid oxidation below that found in healthy individuals without overweight or obesity. For example, the effective amount may be sufficient to effect a reduce the level of fatty acids in adipose tissue. Suitable percent reductions may be a matter of dose and the condition of the subject. The effective amount may vary according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number of binding proteins. The effective amount can be administered in a single dose or in a dose repeated once or several times over a treatment period. For proteins or peptides the effective amount includes from about l0 ug/kg to 20 mg/kg body weight of protein or peptide.

A “therapeutically effective amount” is at least the minimum concentration required to effect a measurable improvement in a subject or population of subject with or at risk of overweight or obesity. A therapeutically effective amount herein may vary according to factors such as the disease state, age, sex, and weight of the patient, and the ability of the modulator to elicit a desired response in the individual. A therapeutically effective amount is also one in which any toxic or detrimental effects of the PSMD9 inhibitor are outweighed by the therapeutically beneficial effects. In one example, a therapeutically effective amount shall be taken to mean a sufficient quantity of PSMD9 inhibitor to reduce or inhibit excess adiposity. As used herein, the term “prophylactically effective amount” shall be taken to mean a sufficient quantity of PSMD9 inhibitor to prevent or inhibit or adipose weight gain in the presence of excess calories.

It will be apparent that “inhibition” of PSMD9 includes partial inhibition such as, for example, by at least about 20% or 30% or 40% or 50% or 60% or 70% or 80% or 90% or 95% inhibition. In some embodiments the PSMD9 inhibitor completely suppresses PSMD9 activity expression in the subject or a cell of the subject for a time and under conditions sufficient to reduce fatty acids in adipose tissue, and/or increase fat break down and utilization such as by increased lipolysis and or lipid oxidation in adipose tissue.

### Peptides and Peptidomimetics

The term “peptide” refers to a sequence of two or more amino acids (e.g. as defined hereinabove) wherein the amino acids are sequentially joined together by amide (peptide) bonds. The sequence may be linear or cyclic. When the sequence is cyclic, the peptide may further comprise other bond types connecting the amino acids, such as an ester bond (a depsipeptide) or a disulfide bond. For example, a cyclic peptide can be prepared or may result from the formation of a disulfide bridge between two cysteine residues in a sequence. Peptide sequences specifically recited herein are written with the amino or N-terminus on the left and the carboxy or C-terminus on the right. A “peptide residue” refers to a sequence of amino acids, that is, amino acids connected by amide bonds, wherein the N-terminus and the C-terminus are not necessarily in free form but may be further linked to additional amino acids or to other radicals. Thus a single peptide may include a large set of possible peptide residues as defined herein. Optionally substituted amino acids and peptides include, although are not limited to phosphoamino acids, phosphopeptides, methylated amino acids, methylated peptides, glycoamino acids, glycopeptides, acylated amino acids, acylated peptides, isoprenylated amino acids, isoprenylated peptides, alkylated amino acids, alkylated peptides, sulfated amino acids, sulfated peptides, glycophosphatidylinositol (GPI anchor) amino acids, glycophosphatidylinositol peptides, ubiquitinated amino acids and ubiquitinated peptides.

Suitable peptides, such as foldamers or stapled peptides, can modulate the level of PSMD9 in a cell, tissue or subject to effect reduced adiposity. In one embodiment, the cell is adipocyte or adipose tissue.

Peptides include phosphopeptides and phosphomimetic peptides. Peptides may be prepared by various synthetic methods known in the art via condensation of one or more amino acids. Peptides may be prepared according to standard solid-phase methods such as may be performed on a peptide synthesizer. Liquid phase methods are also known in the art.

Phosphopeptides may be prepared for example by phosphate assisted peptide ligation. Phophomimetics retain at least one amide bond while others are replaced by an alternative linker, retain or even enhance the biological activity of a peptide for example by reducing enzymatic degradation in vivo leading to longer half-lives which can be advantageous in some embodiments. Peptides with for example phosphomimetic modifications may be readily synthesized by non-fermentation methods.

A peptidomimetic is typically characterised by retaining the polarity, three dimensional size and functionality (bioactivity) of its peptide equivalent but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub., provides a general discussion of prior art techniques for the design and synthesis of peptidomimetics. Suitable amide bond surrogates include the following groups: N-alkylation, retro-inverse amide, thioamide, thioester, phosphonate, ketomethylene, hydroxymethylene, fluorovinyl, vinyl, methyleneamino, methylenethio, alkane and sulfonamido.

Peptides and peptidomimetics will generally have a backbone of 4 to 20, or 7 to 16 amino acids in length. Molecules having backbones at the upper end of these ranges will generally comprise beta and/or gamma amino acids or their equivalents.

### Polypeptide or Polypeptide Fragment PSMD9 Modulators

In some embodiments a PSMD9 inhibitor is a polypeptide inhibitor or antagonist, which may modulate PSMD9 activity by one or more of a number of different mechanisms, for example by specifically binding to PSMD9 or a PSMD9 binding partner thereby reducing interaction of PSMD9 and the binding partner, or, alternatively, competing with PSMD9 for interaction with a binding partner.

In some embodiments a PSMD9 modulator is an antibody or PSMD9-binding fragment thereof that binds to PSMD9 and inhibits its activity. The antibody is generally an antibody modified to penetrate or be taken up (passively or actively) in mammalian cells including adipocytes.

The term “antibody” as used herein includes polyclonal antibodies, monoclonal antibodies, bispecific antibodies, fusion diabodies, triabodies, heteroconjugate antibodies, and chimeric antibodies. Also contemplated are antibody fragments that retain at least substantial (about 10%) antigen binding relative to the corresponding full length antibody. Antibody-based peptides such as linear, monocyclic, bicyclic, stapled or structurally constrained peptides known in the art or polypeptides that penetrate cells of the subject and particularly adipose tissue cells and inhibit PSMD9 expression or activity are expressly contemplated. Such antibody fragments are referred to herein as “antigen-binding fragments”. Antibodies include modifications in a variety of forms including, for example, but not limited to, domain antibodies including either the VH or VL domain, a dimer of the heavy chain variable region (VHH, as described for a camelid), a dimer of the light chain variable region (VLL), Fv fragments containing only the light (VL) and heavy chain (VH) variable regions which may be joined directly or through a linker, or Fd fragments containing the heavy chain variable region and the CHI domain.

A scFv consisting of the variable regions of the heavy and light chains linked together to form a single-chain antibody and oligomers of scFvs such as diabodies and triabodies are also encompassed by the term “antibody”. Also encompassed are fragments of antibodies such as Fab, (Fab′)2 and FabFc2 fragments which contain the variable regions and parts of the constant regions. Complementarity determining region (CDR)-grafted antibody fragments and oligomers of antibody fragments are also encompassed. The heavy and light chain components of an Fv may be derived from the same antibody or different antibodies thereby producing a chimeric Fv region. The antibody may be of animal (for example mouse, rabbit or rat) or human origin or may be chimeric or humanized.

As used herein the term “antibody” includes these various forms. Using the guidelines provided herein and those methods well known to those skilled in the art which are described in the references cited above and in such publications as Harlow & Lane Antibodies: a Laboratory Manual, Cold Spring Harbor Laboratory, (1988) the antibodies for use in the methods of the present invention can be readily made.

The antibodies may be Fv regions comprising a variable light (VL) and a variable heavy (VH) chain in which the light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide. In another embodiment, recombinantly produced single chain scFv antibody, preferably a humanized scFv, is used in the methods of the invention.

In one embodiment, the antibodies have the capacity for intracellular transmission. Antibodies which have the capacity for intracellular transmission include antibodies such as camelids and llama antibodies, shark antibodies (IgNARs), scFv antibodies, intrabodies or nanobodies, for example, scFv intrabodies and VHH intrabodies. Yeast SPLINT antibody libraries are available for testing for intrabodies which are able to disrupt protein-protein interactions. Such agents may comprise a cell-penetrating peptide sequence or nuclear-localizing peptide sequence such as those disclosed in Constantini et al. (2008). Also useful for in vivo delivery are Vectocell or Diato peptide vectors such as those disclosed in De Coupade et al. (2005).

In addition, the antibodies may be fused to a cell penetrating agent, for example a cell-penetrating peptide. Cell penetrating peptides include Tat peptides, Penetratin, short amphipathic peptides such as those from the Pep- and MPG-families, oligoarginine and oligolysine. In one example, the cell penetrating peptide is also conjugated to a lipid (C6-C 1 8 fatty acid) domain to improve intracellular delivery (Koppelhus et al., 2008). Examples of cell penetrating peptides are known in the art. Thus, the invention also provides the therapeutic use of antibodies fused via a covalent bond (e.g. a peptide bond), at optionally the N-terminus or the C-terminus, to a cell-penetrating peptide sequence.

Antibodies which specifically target mammalian PSMD9 are available from various commercial sources known to the skilled addressee.

In one embodiment the PSMD9 inhibitor is an antibody or an antibody fragment or an antibody mimic as known in the art, such as a bicycle, an Fv, scFv, di-scFv, diabody, triabody, tetrabody, Fab, F(ab′)2, bispecific antibodies, full length antibody, chimeric, human etc antibody. Non-Ig binding proteins include monobodies, anticalins, and Darpins, LoopDarbins affibodies (Jost et al. Current opinion in Structural Biology 2014 27:102-112). Synthetic antibody mimetics are also contemplated, as are ibodies (Adalta). A multitude of antibody fragments or derivatives comprising an antibody variable region able to bind to precise proteins are also known to the skilled addressee.

### Small Molecule PSMD9 Modulators

PDSM9 modulators may be small molecules. Small molecules are molecules having a molecular mass less than 2000 daltons. Small molecules may be in the form of pro-drugs or active metabolites. Small molecules may be used in the form of a salt wherein the counter ion is pharmaceutically or physiologically acceptable. Suitable salts are known in the art. The skilled person will understand the use of small molecules in the form of solvates such as hydrates. Small molecules may also be in amorphous or crystalline form.

Small molecules useful for the present application of down modulating PSMD9 can be identified using standard procedures, such as without limitation screening a library of candidate compounds for binding to PSMD9 and then determining whether any of the compounds which bind to PSMD9 also down modulate PSMD9 activity or binding. In silico modelling of compounds can also be useful as can high throughput chemical screening, functional based assays or structure activity relationships.

Small molecules, peptides etc and other agents can be screened by competitive fluorescence polarization binding assays and then progress to more selective quantitation of PSMD9 inhibition, binding and specificity. Activity studies may be conducted using dilutions of agents and in vitro or in vivo screens for their ability to modulate lipid metabolism. Such screens, identified herein or known in the art are applied in vivo and used to test and develop candidate agents and determine their stability and toxicity, bioavailability etc. Thus, the term “in the manufacture of a medicament” encompasses in vitro and in vivo screening and development. Natural products, combinatorial synthetic organic or inorganic compounds, fragment libraries, peptide/polypeptide/protein, nucleic acid molecules and libraries or phage or other display technology comprising these are all available to screen or test for suitable agents.

Natural products include those from coral, soil, plant, or the ocean or Antarctic environments. Libraries of small organic molecules can be generated and screened using high-throughput technologies known to those of skill in this art. See for example U.S. Pat. No. 5,763,623 and United States Application No. 20060167237. Combinatorial synthesis provides a very useful approach wherein a great many related compounds are synthesized having different substitutions of a common or subset of parent structures. Such compounds are usually non-oligomeric and may be similar in terms of their basic structure and function, for example, varying in chain length, ring size or number or substitutions. Virtual libraries are also contemplated and these may be constructed and compounds tested in silico (see for example, US Publication No. 20060040322) or by in vitro or in vivo assays known in the art. Libraries of small molecules suitable for testing are available in the art (see for example, Amezcua et al., Structure London), 10: 1349-1361, 2002). Yeast SPLINT antibody libraries are available for testing for intrabodies which are able to disrupt protein-protein interactions (see Visintin et al., supra). Examples of suitable methods for the synthesis of molecular libraries can be found in the art. Bicyclic peptides are recently described in Liskamp Nature Chemistry 6, 855-857 2014. Agents may be hydrocarbon-stapled peptides or miniature proteins which are alpha-helical and cell-penetrating, and are able to disrupt protein-protein interactions (see for example, Wilder et al., Chem Med Chem. 2(8): 1149-1151, 2007; & for a review see, Henchey et al., Curr. Opin. Chem. Biol., 2(6):692-697, 2008. See also U.S. Publication No. 2005/0250680.

Thus, agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is suited to peptide libraries, while the other four approaches are applicable to peptide, non-peptide oligomer or small molecule libraries of compounds. Libraries of compounds may be presented, for example, in solution, or on beads, chips, bacteria, spores and plasmids or phage as known in the art.

In one embodiment a small molecule PSMD9 modulator is a reversible or an irreversible inhibitor of PSMD9.

In one embodiment, a small molecule PSMD9 modulator is an inhibitor of the expression of PSMD9.

### Oligonucleotide PSMD9 Modulators/Inhibitors

In one embodiment the present disclosure enables the use of an antisense compound to PSMD9. Such antisense compounds are targeted to nucleic acids encoding the PSMD9. In one embodiment, the antisense compound is an oligonucleotide. However, other oligomeric antisense compounds, including but not limited to oligonucleotide mimetics are contemplated.

In certain embodiments, compounds described herein are antisense compounds. In certain embodiments, the antisense compound comprises or consists of an oligomeric compound. In certain embodiments, the oligomeric compound comprises a modified oligonucleotide. In certain embodiments, the modified oligonucleotide has a nucleobase sequence complementary to that of a target nucleic acid. In certain embodiments, a compound described herein comprises or consists of a modified oligonucleotide. In certain embodiments, the modified oligonucleotide has a nucleobase sequence complementary to that of a target nucleic acid.

Examples of single-stranded and double-stranded compounds include but are not limited to oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, a compound described herein has a nucleobase sequence that, when written in the 5′ to 3′ direction, comprises the reverse complement of the target segment of a target nucleic acid to which it is targeted.

Hybridization of an antisense compound with its target nucleic acid is generally referred to as “antisense”. Hybridization of the antisense compound with its target nucleic acid inhibits the function of the target nucleic acid. Such “antisense inhibition” is typically based upon hydrogen bonding-based hybridization of the antisense compound to the target nucleic acid such that the target nucleic acid is cleaved, degraded, or otherwise rendered inoperable. The functions of target DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA.

“Hybridization” as used herein means pairing of complementary bases of the oligonucleotide and target nucleic acid. Base pairing typically involves hydrogen bonding, which may be Watson-Crick, Hoogsteen or reversed Hoogsteen hydrogen bonding, between complementary nucleoside or nucleotide bases (nucleobases). Guanine (G) and cytosine (C) are examples of complementary nucleobases which pair through the formation of 3 hydrogen bonds. Adenine (A) and thymine (T) are examples of complementary nucleobases which pair through the formation of 2 hydrogen bonds. Hybridization can occur under varying circumstances.

A “nucleoside” is a base-sugar combination. The base portion of the nucleoside is normally a heterocyclic base. The two most common classes of such heterocyclic bases are the purines and the pyrimidines. “Nucleotides” are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar.

“Specifically hybridizable” and “complementary” are terms which are used to indicate a sufficient degree of complementarity such that stable and specific binding occurs between the antisense compound and target nucleic acid. It is understood that the antisense compound need not be 100% complementary to its target nucleic acid sequence to be specifically hybridizable. An antisense compound is specifically hybridizable when binding of the antisense compound to the target nucleic acid interferes with the normal function of the target molecule to cause a loss of activity, and there is a sufficient degree of complementarity to avoid non-specific binding of the antisense compound to non-target sequences under conditions in which specific binding is desired, for example, under physiological conditions in the case of therapeutic treatment.

“Complementary” as used herein, refers to the capacity for precise pairing between a nucleobase of the antisense compound and the target nucleic acid. For example, if a nucleobase at a certain position of the antisense compound is capable of hydrogen bonding with a nucleobase at a certain position of the target nucleic acid, then the position of hydrogen bonding between the antisense compound and the target nucleic acid is considered to be a complementary position. The antisense compound may hybridize over one or more segments, such that intervening or adjacent segments are not involved in the hybridization event (e.g., a loop structure or hairpin structure). In one embodiment, the antisense compound comprises at least 70% sequence complementarity to a target region within the target nucleic acid.

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to adenine (A) and thymine (T), adenine (A) and uracil (U), cytosine (C) and guanine (G), and 5-methyl cytosine (mC) and guanine (G) unless otherwise specified. Complementary oligonucleotides and/or nucleic acids need not have nucleobase complementarity at each nucleoside and may include one or more nucleobase mismatches. An oligonucleotide is fully complementary or 100% complementary when such oligonucleotides have nucleobase matches at each nucleoside without any nucleobase mismatches.

In certain embodiments, compounds described herein comprise or consist of modified oligonucleotides. In certain embodiments, compounds described herein are antisense compounds. In certain embodiments, compounds comprise oligomeric compounds. Non-complementary nucleobases between a compound and a PSMD9 nucleic acid may be tolerated provided that the compound remains able to specifically hybridize to a target nucleic acid. Moreover, a compound may hybridize over one or more segments of a PSMD9 nucleic acid such that intervening or adjacent segments are not involved in the hybridization event (e.g., a loop structure, mismatch or hairpin structure).

For example, an antisense compound in which 18 of 20 nucleobases are complementary to a target region within the target nucleic acid, and would therefore specifically hybridize, would represent 90% complementarity. In this example, the remaining noncomplementary nucleobases may be clustered or interspersed with complementary nucleobases and need not be contiguous to each other, or to complementary nucleobases. As such, an antisense compound which is 18 nucleobases in length having 4 non-complementary nucleobases which are flanked by 2 regions of complete complementarity with the target nucleic acid would have 77.8% overall complementarity with the target nucleic acid and would thus, fall within the scope of the present disclosure. Percent complementarity of an antisense compound with a region of a target nucleic acid can be determined routinely using BLAST programs (basic local alignment search tools) and PowerBLAST programs known in the art (Altschul et al., 1990; Zhang and Madden, 1997).

In certain embodiments, the compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a PSMD9 nucleic acid, a target region, target segment, or specified portion thereof.

In certain embodiments, compounds described herein, or specified portions thereof, are fully complementary (i.e. 100% complementary) to a target nucleic acid, or specified portion thereof. For example, a compound may be fully complementary to a PSMD9 nucleic acid, or a target region, or a target segment or target sequence thereof. As used herein, “fully complementary” means each nucleobase of a compound is capable of precise base pairing with the corresponding nucleobases of a target nucleic acid. For example, a 20 nucleobase compound is fully complementary to a target sequence that is 400 nucleobases long, so long as there is a corresponding 20 nucleobase portion of the target nucleic acid that is fully complementary to the compound. Fully complementary can also be used in reference to a specified portion of the first and/or the second nucleic acid. For example, a 20 nucleobase portion of a 30 nucleobase compound can be “fully complementary” to a target sequence that is 400 nucleobases long. The 20 nucleobase portion of the 30 nucleobase compound is fully complementary to the target sequence if the target sequence has a corresponding 20 nucleobase portion wherein each nucleobase is complementary to the 20 nucleobase portion of the compound. At the same time, the entire 30 nucleobase compound may or may not be fully complementary to the target sequence, depending on whether the remaining 10 nucleobases of the compound are also complementary to the target sequence.

In certain embodiments, compounds described herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of a compound. In certain embodiments, the compounds are complementary to at least an 8 nucleobase portion of a target segment. In certain embodiments, the compounds are complementary to at least a 9 nucleobase portion of a target segment. In certain embodiments, the compounds are complementary to at least a 10 nucleobase portion of a target segment. In certain embodiments, the compounds are complementary to at least an 11 nucleobase portion of a target segment. In certain embodiments, the compounds are complementary to at least a 12 nucleobase portion of a target segment. In certain embodiments, the compounds are complementary to at least a 13 nucleobase portion of a target segment. In certain embodiments, the compounds are complementary to at least a 14 nucleobase portion of a target segment. In certain embodiments, the compounds are complementary to at least a 15 nucleobase portion of a target segment. In certain embodiments, the compounds are complementary to at least a 16 nucleobase portion of a target segment. Also contemplated are compounds that are complementary to at least a 9, 10, 11, 12, 13, 14, 15, 16, 17, 18, 19, 20, or more nucleobase portion of a target segment, or a range defined by any two of these values.

In some embodiments, the antisense molecule is substantially identical with at least a region of the coding sequence of the target gene to enable down-regulation of the gene. In some embodiments, the degree of identity between the sequence of the antisense molecule and the targeted region of the gene is at least 60% sequence identity, in some embodiments at least 75% sequence identity, for instance at least 85% identity, 90% identity, at least 95% identity, at least 97%, or at least 99% identity.

Calculation of percentage identities between different amino acid/polypeptide/nucleic acid sequences may be carried out as follows. A multiple alignment is first generated by the ClustalX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared.

Alternatively, percentage identity can be calculated as (N/S)*100 where S is the length of the shorter sequence being compared. The amino acid/polypeptide/nucleic acid sequences may be synthesized de novo, or may be native amino acid/polypeptide/nucleic acid sequence, or a derivative thereof. A substantially similar nucleotide sequence will be encoded by a sequence which hybridizes to any of the nucleic acid sequences referred to herein or their complements under stringent conditions. By stringent conditions, we mean the nucleotide hybridizes to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change.

For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid. The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA. Frequently, automatically generated alignments require manual alignment, exploiting the trained user's knowledge of the protein family being studied, e.g., biological knowledge of key conserved sites. One such alignment editor programs is Align (http://www.gwdg.de/dhepper/download/; Hepperle, D., 2001: Multicolor Sequence Alignment Editor. Institute of Freshwater Ecology and Inland Fisheries, 1 6775 Stechlin, Germany), although others, such as JalView or Cinema are also suitable. Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs. washington.edu/phylip.html) using the “Similarity Table” option as the model for amino acid substitution (P). For DNA/RNA, an identical option exists within the DNADIST program of PHYL1 P.

The molecules may comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. In an embodiment, the NA molecules specifically target one given gene. In order to only target the desired mRNA, the antisense reagent may have 1 00% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

The length of the region of the antisense complementary to the target, in accordance with the present invention, may be from 10 to 100 nucleotides, 12 to 25 nucleotides, 14 to 22 nucleotides or 15, 16, 17 or 18 nucleotides.

In certain embodiments, a compound described herein comprises an oligonucleotide 10 to 30 linked subunits in length. In certain embodiments, compound described herein comprises an oligonucleotide is 12 to 30 linked subunits in length. In certain embodiments, compound described herein comprises an oligonucleotide is 12 to 22 linked subunits in length. In certain embodiments, compound described herein comprises an oligonucleotide is 14 to 30 linked subunits in length. In certain embodiments, compound described herein comprises an oligonucleotide is 14 to 20 linked subunits in length. In certain embodiments, compound described herein comprises an oligonucleotide is 15 to 30 linked subunits in length. In certain embodiments, compound described herein comprises an oligonucleotide is 15 to 20 linked subunits in length. In certain embodiments, compound described herein comprises an oligonucleotide is 16 to 30 linked subunits in length. In certain embodiments, compound described herein comprises an oligonucleotide is 16 to 20 linked subunits in length. In certain embodiments, compound described herein comprises an oligonucleotide is 17 to 30 linked subunits in length. In certain embodiments, compound described herein comprises an oligonucleotide is 17 to 20 linked subunits in length. In certain embodiments, compound described herein comprises an oligonucleotide is 18 to 30 linked subunits in length. In certain embodiments, compound described herein comprises an oligonucleotide is 18 to 21 linked subunits in length. In certain embodiments, compound described herein comprises an oligonucleotide is 18 to 20 linked subunits in length. In certain embodiments, compound described herein comprises an oligonucleotide is 20 to 30 linked subunits in length. In other words, such oligonucleotides are from 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, 18 to 21 subunits, 20 to 30 subunits, or 12 to 22 linked subunits, respectively. In certain embodiments, a compound described herein comprises an oligonucleotide 14 linked subunits in length. In certain embodiments, a compound described herein comprises an oligonucleotide 16 linked subunits in length. In certain embodiments, a compound described herein comprises an oligonucleotide 17 linked subunits in length. In certain embodiments, compound described herein comprises an oligonucleotide 18 linked subunits in length. In certain embodiments, a compound described herein comprises an oligonucleotide 19 linked subunits in length. In certain embodiments, a compound described herein comprises an oligonucleotide 20 linked subunits in length. In other embodiments, a compound described herein comprises an oligonucleotide 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the compound described herein comprises an oligonucleotide 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the linked subunits are nucleotides, nucleosides, or nucleobases.

Where there are mismatches to the corresponding target region, the length of the complementary region is generally required to be somewhat longer. In an embodiment, the inhibitor is a siRNA molecule and comprises between approximately 5 bp and 50 bp, in some embodiments, between 10 bp and 35 bp, or between 15 bp and 30 bp, for instance between 18 bp and 25 bp. In some embodiments, the siRNA molecule comprises more than 20 and less than 23 bp.

In certain embodiments, compounds described herein are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics.

In certain embodiments, a double-stranded compound comprises a first strand comprising the nucleobase sequence complementary to a target region of a PSMD9 nucleic acid and a second strand. In certain embodiments, the double-stranded compound comprises ribonucleotides in which the first strand has uracil (U) in place of thymine (T) and is complementary to a target region. In certain embodiments, a double-stranded compound comprises (i) a first strand comprising a nucleobase sequence complementary to a target region of a PSMD9 nucleic acid, and (ii) a second strand. In certain embodiments, the double-stranded compound comprises one or more modified nucleotides in which the 2′ position in the sugar contains a halogen (such as fluorine group; 2′-F) or contains an alkoxy group (such as a methoxy group; 2′-OMe). In certain embodiments, the double-stranded compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the dsRNA compound. In certain embodiments, the double-stranded compound comprises one or more linkages between adjacent nucleotides other than a naturally-occurring phosphodiester linkage. Examples of such linkages include phosphoramide, phosphorothioate, and phosphorodithioate linkages. The double-stranded compounds may also be chemically modified nucleic acid molecules as taught in U.S. Pat. No. 6,673,661. In other embodiments, the dsRNA contains one or two capped strands, as disclosed, for example, by WO 00/63364, filed Apr. 19, 2000. In certain embodiments, the first strand of the double-stranded compound is an siRNA guide strand and the second strand of the double-stranded compound is an siRNA passenger strand. In certain embodiments, the second strand of the double-stranded compound is complementary to the first strand. In certain embodiments, each strand of the double-stranded compound consists of 16, 17, 18, 19, 20, 21, 22, or 23 linked nucleosides.

In certain embodiments, a single-stranded compound described herein can comprise any of the oligonucleotide sequences targeted to PSMD9 described herein. In certain embodiments, such a single-stranded compound is a single-stranded RNAi (ssRNAi) compound. In certain embodiments, a ssRNAi compound comprises the nucleobase sequence complementary to a target region of a PSMD9 nucleic acid. In certain embodiments, the ssRNAi compound comprises ribonucleotides in which uracil (U) is in place of thymine (T).

In certain embodiments, ssRNAi compound comprises a nucleobase sequence complementary to a target region of a PSMD9 nucleic acid. In certain embodiments, a ssRNAi compound comprises one or more modified nucleotides in which the 2′ position in the sugar contains a halogen (such as fluorine group; 2′-F) or contains an alkoxy group (such as a methoxy group; 2′-OMe). In certain embodiments, a ssRNAi compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the ssRNAi compound. In certain embodiments, the ssRNAi compound comprises one or more linkages between adjacent nucleotides other than a naturally-occurring phosphodiester linkage. Examples of such linkages include phosphoramide, phosphorothioate, and phosphorodithioate linkages. The ssRNAi compounds may also be chemically modified nucleic acid molecules as taught in U.S. Pat. No. 6,673,661. In other embodiments, the ssRNAi contains a capped strand, as disclosed, for example, by WO 00/63364, filed Apr. 19, 2000. In certain embodiments, the ssRNAi compound consists of 16, 17, 18, 19, 20, 21, 22, or 23 linked nucleosides.

Because the siRNA may carry overhanging ends (which may or may not be complementary to the target), or additional nucleotides complementary to itself but not the target gene, the total length of each separate strand of siRNA may be 10 to 1 00 nucleotides, 15 to 49 nucleotides, 17 to 30 nucleotides or 1 9 to 25 nucleotides. The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand. The siRNA according to the present invention display a high in vivo stability and may be particularly suitable for oral delivery by including at least one modified nucleotide in at least one of the strands.

In certain embodiments, compounds described herein comprise modified oligonucleotides. Certain modified oligonucleotides have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L) such as for amino acids etc. Included in the modified oligonucleotides provided herein are all such possible isomers, including their racemic and optically pure forms, unless specified otherwise. Likewise, all cis- and trans-isomers and tautomeric forms are also included.

The term “microRNA” (abbreviated miRNA) is a small non-coding RNA molecule (containing about 22 nucleotides) found in plants, animals and some viruses, that functions in RNA silencing and post-transcriptional regulation of gene expression. The prefix “miR” is followed by a dash and a number, the latter often indicating order of naming. Different miRNAs with nearly identical sequences except for one or two nucleotides are annotated with an additional lower case letter. Numerous miRNAs are known in the art (miRBase V.21 nomenclature.

In one embodiment, modulatory oligonucleotides mimic the activity of one or more miRNA. The term “miRNA mimic”, as used herein, refers to small, double-stranded RNA molecules designed to mimic endogenous mature miRNA molecules when introduced into cells. miRNA mimics can be obtained from various suppliers such as Sigma Aldrich and Thermo Fisher Scientific.

In one embodiment, modulatory oligonucleotides inhibit the activity of one or more miRNA. Various miRNA species are suitable for this purpose. Examples include, without limitation, antagomirs, interfering RNA, ribozymes, miRNA sponges and miR-masks. The term “antagomir” is used in the context of the present disclosure to refer to chemically modified antisense oligonucleotides that bind to a target miRNA and inhibit miRNA function by preventing binding of the miRNA to its cognate gene target. Antagomirs can include any base modification known in the art. In an example, the above referenced miRNA species are about 10 to 50 nucleotides in length. For example, antagomirs can have antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In one embodiment, modulatory oligonucleotides are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids.

In one embodiment, modulatory oligonucleotides are synthetic. The term “synthetic nucleic acid” means that the nucleic acid does not have a chemical structure or sequence of a naturally occurring nucleic acid. Synthetic nucleotides include an engineered nucleic acid such as a DNA or RNA molecule. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA. Consequently, it will be understood that the term “synthetic miRNA” refers to a “synthetic nucleic acid” that functions in a cell or under physiological conditions as a naturally occurring miRNA. In another example, the nucleic acid structure can also be modified into a locked nucleic acid (LNA) with a methylene bridge between the 2′ Oxygen and the 4′ carbon to lock the ribose in the 3′-endo (North) conformation in the A-type conformation of nucleic acids. In the context of miRNAs, this modification can significantly increase both target specificity and hybridization properties of the molecule.

Nucleic acids for use in the methods disclosed herein can be designed using routine methods as required. For example, in the context of inhibitory oligonucleotides, target segments of 5, 6, 7, 8, 9, 10 or more nucleotides in length comprising a stretch of at least five (5) consecutive nucleotides within the seed sequence, or immediately adjacent thereto, are considered to be suitable for targeting a gene. Exemplary target segments can include sequences that comprise at least the 5 consecutive nucleotides from the 5′-terminus of one of the seed sequence (the remaining nucleotides being a consecutive stretch of the same RNA beginning immediately upstream of the 5′-terminus of the seed sequence and continuing until the nucleic acid contains about 5 to about 30 nucleotides). In another example, target segments are represented by RNA sequences that comprise at least the 5 consecutive nucleotides from the 3′-terminus of one of the seed sequence (the remaining nucleotides being a consecutive stretch of the same RNA beginning immediately downstream of the 3′-terminus of the target segment and continuing until the nucleic acid contains about 5 to about 30 nucleotides). The term “seed sequence” is used in the context of the present disclosure to refer to a 6-8 nucleotide (nt) long substring within the first 8 nt at the 5-end of the miRNA (i.e., seed sequence) that is an important determinant of target specificity. Once one or more target regions, segments or sites have been identified, inhibitory nucleic acid compounds are chosen that are sufficiently complementary to the target, i.e., that hybridize sufficiently well and with sufficient specificity (i.e., do not substantially bind to other non-target nucleic acid sequences), to give the desired effect.

Various online tools are available providing software and guidelines for designing RNAi/siRNA, for example Thermo Fisher, GeneScript, InvivoGen, and the siDESIGN tool. These are then tested empirically with typically at least 3 out of 10 siRNAs anticipated to result in mRNA knockdown rate of at least 75% where the transfection efficiency is at least 80%. Reference may be made to WO2005054270 and US20030186909.

### Antisense Oligonucleotides

The present disclosure provides antisense oligonucleotides for inhibiting expression of PSMD9. Such antisense oligonucleotides are targeted to nucleic acids encoding PSMD9.

The term “inhibits” as used herein means any measurable decrease (e.g., 10%, 20%, 50%, 90%, or 100%) in PSMD9 expression.

As used herein, the term “oligonucleotide” refers to an oligomer or polymer of RNA or DNA or mimetics, chimeras, analogs and homologs thereof. This term includes oligonucleotides composed of naturally occurring nucleobases, sugars and covalent internucleoside (backbone) linkages, as well as oligonucleotides having non-naturally occurring portions which function similarly. Such modified or substituted oligonucleotides are often preferred over native forms because of desirable properties such as, for example, enhanced cellular uptake, enhanced affinity for the target nucleic acid and increased stability in the presence of nucleases.

The oligonucleotides may contain chiral (asymmetric) centers or the molecule as a whole may be chiral. The individual stereoisomers (enantiomers and diastereoisomers) and mixtures of these are within the scope of the present disclosure. Reference may be made to Wan et al. Nucleic Acids Research 42 (22:13456-13468, 2014 for a disclosure of antisense oligonucleotides containing chiral phosphorothioate linkages.

In forming oligonucleotides, phosphate groups covalently link adjacent nucleosides to one another to form a linear polymeric compound. In turn, the respective ends of this linear polymeric compound can be further joined to form a circular compound; however, linear compounds are generally preferred. In addition, linear compounds may have internal nucleobase complementarity and may therefore fold in a manner so as to produce a fully or partially double-stranded compound. With regard to oligonucleotides, the phosphate groups are commonly referred to as forming the internucleoside backbone of the oligonucleotide. The normal linkage or backbone of RNA and DNA is a 3′ to 5′ phosphodiester linkage.

Antisense oligonucleotides of the disclosure include, for example, ribozymes, siRNA, external guide sequence (EGS) oligonucleotides, alternate splicers, primers, probes, and other oligonucleotides which hybridize to at least a portion of the target nucleic acid.

Antisense oligonucleotides of the disclosure may be administered in the form of single-stranded, double-stranded, circular or hairpin and may contain structural elements such as internal or terminal bulges or loops. Once administered, the antisense oligonucleotides may elicit the action of one or more enzymes or structural proteins to effect modification of the target nucleic acid.

One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H therefore results in cleavage of the RNA target, thereby greatly enhancing the efficiency of oligonucleotide-mediated inhibition of gene expression. Similar roles have been postulated for other ribonucleases, such as those in the RNase III and ribonuclease L family of enzymes. Further, in certain embodiments, one or more non-DNA-like nucleoside in the gap of a gapmer is tolerated.

The introduction of double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

In certain antisense activities, compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

The first evidence that dsRNA could lead to gene silencing in animals came in 1995 from work in the nematode, Caenorhabditis elegans. Others have shown that the primary interference effects of dsRNA are posttranscriptional. The post-transcriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels. More recently, it has been shown that it is, in fact, the single-stranded RNA oligomers of antisense polarity of the dsRNAs which are the potent inducers of RNAi (Tijsterman et al., 2002).

A person having ordinary skill in the art could, without undue experimentation, identify antisense oligonucleotides useful in the methods of the present disclosure.

### Modified Internucleoside Linkages (Backbones)

The naturally occurring internucleoside linkage of RNA and DNA is a 3′ to 5′ phosphodiester linkage. In certain embodiments, compounds described herein having one or more modified, i.e. non-naturally occurring, internucleoside linkages are often selected over compounds having naturally occurring internucleoside linkages because of desirable properties such as, for example, enhanced cellular uptake, enhanced affinity for target nucleic acids, and increased stability in the presence of nucleases. Antisense compounds of the present disclosure include oligonucleotides having modified backbones or non-natural internucleoside linkages. In certain embodiments, compounds targeted to a PSMD9 nucleic acid comprise one or more modified internucleoside linkages. In certain embodiments, the modified internucleoside linkages are phosphorothioate linkages. In certain embodiments, each internucleoside linkage of the compound is a phosphorothioate internucleoside linkage. Oligonucleotides having modified backbones include those that retain a phosphorus atom in the backbone and those that do not have a phosphorus atom in the backbone.

In certain embodiments, compounds described herein comprise oligonucleotides. Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

Modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage, that is, a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808, 4,469,863, 4,476,301, 5,023,243, 5,177,196, 5,188,897, 5,264,423, 5,276,019, 5,278,302, 5,286,717, 5,321,131, 5,399,676, 5,405,939, 5,453,496, 5,455,233, 5,466,677, 5,476,925, 5,519,126, 5,536,821, 5,541,306, 5,550,111, 5,563,253, 5,571,799, 5,587,361, 5,194,599, 5,565,555, 5,527,899, 5,721,218, 5,672,697 and 5,625,050.

Modified oligonucleotide backbones that do not include a phosphorus atom therein include, for example, backbones formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones;

riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative United States patents that teach the preparation of the above oligonucleotides include, but are not limited to, U.S. Pat. Nos. 5,034,506, 5,166,315, 5,185,444, 5,214,134, 5,216,141, 5,235,033, 5,264,562, 5,264,564, 5,405,938, 5,434,257, 5,466,677, 5,470,967, 5,489,677, 5,541,307, 5,561,225, 5,596,086, 5,602,240, 5,610,289, 5,602,240, 5,608,046, 5,610,289, 5,618,704, 5,623,070, 5,663,312, 5,633,360, 5,677,437, 5,792,608, 5,646,269 and 5,677,439.

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The nucleoside motif is independently selected, so such oligonucleotides having a gapped internucleoside linkage motif may or may not have a gapped nucleoside motif and if it does have a gapped nucleoside motif, the wing and gap lengths may or may not be the same.

In certain embodiments, oligonucleotides comprise a region having an alternating internucleoside linkage motif. In certain embodiments, oligonucleotides of the present invention comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

### Modified Sugar and Internucleoside Linkages

Antisense compounds of the present disclosure include oligonucleotide mimetics where both the sugar and the internucleoside linkage (i.e. the backbone), of the nucleotide units are replaced with novel groups. The nucleobase units are maintained for hybridization with the target nucleic acid.

An oligonucleotide mimetic that has been shown to have excellent hybridization properties is referred to as a peptide nucleic acid (PNA). In PNA compounds, the sugar-backbone of an oligonucleotide is replaced with an amide containing backbone, in particular, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082, 5,714,331, and 5,719,262. The antisense compounds of the present disclosure also include oligonucleotides with phosphorothioate backbones and oligonucleotides with heteroatom backbones, for example, —CH2—NH—O—CH2-, —CH2-N(CH3)-O—CH2- [known as a methylene (methylimino) or MMI backbone], —CH2-O—N(CH3)-CH2-, -CH2-N(CH3)-N(CH3)-CH2- and —O—N(CH3)-CH2-CH2- [wherein the native phosphodiester backbone is represented as —O—P—O—CH2-] of U.S. Pat. No. 5,489,677, and the amide backbones of U.S. Pat. No. 5,602,240.

The antisense compounds of the present disclosure also include oligonucleotides having morpholino backbone structures of U.S. Pat. No. 5,034,506.

### Modified Sugars

Antisense compounds of the present disclosure include oligonucleotides having one or more substituted sugar moieties. In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

Examples include oligonucleotides comprising one of the following at the 2′ position: OH; F; O-, S-, or N-alkyl; O—, S-, or N-alkenyl; O—, S- or N-alkynyl; or O-alkyl-O-alkyl, wherein the alkyl, alkenyl and alkynyl may be substituted or unsubstituted C1 to C10 alkyl or C2 to C10 alkenyl and alkynyl.

In one embodiment, the oligonucleotide comprises one of the following at the 2′ position: O[(CH2)nO]mCH3, O(CH2)nOCH3, O(CH2)nNH2, O(CH2)nCH3, O(CH2)nONH2, and O(CH2)nON[(CH2)nCH3]2, where n and m are from 1 to about 10.

Further examples include of modified oligonucleotides include oligonucleotides comprising one of the following at the 2′ position: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

In one embodiment, the modification includes 2′-methoxyethoxy (2′-O—CH2CH2OCH3 (also known as 2′-O-(2-methoxyethyl) or 2′-MOE), that is, an alkoxyalkoxy group. In a further embodiment, the modification includes 2′-dimethylaminooxyethoxy, that is, a O(CH2)2N(CH3)2 group (also known as 2′-DMAOE), or 2′-dimethylaminoethoxyethoxy (also known in the art as 2′-O-dimethyl-amino-ethoxy-ethyl or 2′-DMAEOE), that is, 2′-O-CH2-O-CH2-N(CH3)2.

Other modifications include 2′-methoxy (2′-O—CH3), 2′-aminopropoxy (2′-OCH2CH2CH2NH2), 2′ allyl (2′-CH2—CH═CH2), (2′-O—CH2—CH═CH2) and 2′-fluoro (2′-F). The 2′-modification may be in the arabino (up) position or ribo (down) position. In one embodiment a 2′-arabino modification is 2′-F.

Similar modifications may also be made at other positions on the oligonucleotide, particularly the 3′ position of the sugar on the 3′ terminal nucleotide or in 2′-5′ linked oligonucleotides and the 5′ position of the 5′ terminal nucleotide.

Oligonucleotides may also have sugar mimetics, such as cyclobutyl moieties in place of the pentofuranosyl sugar.

Representative United States patents that teach the preparation of such modified sugar structures include, but are not limited to, U.S. Pat. Nos. 4,981,957, 5,118,800, 5,319,080, 5,359,044, 5,393,878, 5,446,137, 5,466,786, 5,514,785, 5,519,134, 5,567,811, 5,576,427, 5,591,722, 5,597,909, 5,610,300, 5,627,053, 5,639,873, 5,646,265, 5,658,873, 5,670,633, 5,792,747, and 5,700,920.

A further modification of the sugar includes Locked Nucleic Acids (LNAs) in which the 2′-hydroxyl group is linked to the 3′ or 4′ carbon atom of the sugar ring, thereby forming a bicyclic sugar moiety. In one embodiment, the linkage is a methylene (—CH2-)n group bridging the 2′ oxygen atom and the 4′ carbon atom, wherein n is 1 or 2. LNAs and preparation thereof are described in WO 98/39352 and WO 99/14226.

Certain modifed sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, (“LNA”), 4′-(CH₂)₂—O-2′ (“ENA”), 4′-CH(CH₃)—O-2′ (referred to as “constrained ethyl” or “cEt” when in the S configuration), 4′-CH2-O-CH2-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH2-C(H)(CH3)-2′ (see, e.g., Zhou, et al., J. Org. Chem., 2009, 74, 118-134), 4′-CH2-C—(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

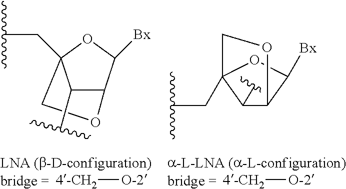

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

### Natural and Modified Nucleobases

Antisense compounds of the present disclosure include oligonucleotides having nucleobase modifications or substitutions. As used herein, “unmodified” or “natural” nucleobases include the purine bases adenine (A) and guanine (G), and the pyrimidine bases thymine (T), cytosine (C) and uracil (U). Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to compounds described herein.

Modified nucleobases include other synthetic and natural nucleobases such as, for example, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C—C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Further modified nucleobases include tricyclic pyrimidines, such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as, for example, a substituted phenoxazine cytidine (e.g., 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one).

Modified nucleobases may also include those in which the purine or pyrimidine base is replaced with other heterocycles, for example, 7-deaza-adenine, 7-deazaguanosine, 2-aminopyridine and 2-pyridone. Further nucleobases include those disclosed in U.S. Pat. No. 3,687,808, those disclosed in J. I. Kroschwitz (editor), The Concise Encyclopedia of Polymer Science and Engineering, pages 858-859, John Wiley and Sons (1990), those disclosed by Englisch et al. (1991), and those disclosed by Y. S. Sanghvi, Chapter 15: Antisense Research and Applications, pages 289-302, S. T. Crooke, B. Lebleu (editors), CRC Press, 1993.

Certain of these nucleobases are particularly useful for increasing the binding affinity of the oligonucleotide. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. In one embodiment, these nucleobase substitutions are combined with 2′-O-methoxyethyl sugar modifications.

Representative United States patents that teach the preparation of certain of the above noted modified nucleobases as well as other modified nucleobases include, but are not limited to, U.S. Pat. Nos. 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096, 5,681,941 and 5,750,692.

In certain embodiments, compounds targeted to a PSMD9 nucleic acid comprise one or more modified nucleobases. In certain embodiments, the modified nucleobase is 5-methylcytosine. In certain embodiments, each cytosine is a 5-methylcytosine.

### Conjugates

Antisense compounds of the present disclosure may be conjugated to one or more moieties or groups which enhance the activity, cellular distribution or cellular uptake of the antisense compound.

These moieties or groups may be covalently bound to functional groups such as primary or secondary hydroxyl groups.

Exemplary moieties or groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins and dyes.

Moieties or groups that enhance the pharmacodynamic properties include those that improve uptake, enhance resistance to degradation, and/or strengthen sequence-specific hybridization with the target nucleic acid.

Moieties or groups that enhance the pharmacokinetic properties include those that improve uptake, distribution, metabolism or excretion of the compounds of the present disclosure. Representative moieties or groups are disclosed in PCT/US92/09196 and U.S. Pat. No. 6,287,860. Moieties or groups include but are not limited to lipid moieties such as a cholesterol moiety, cholic acid, a thioether, for example, hexyl-S-tritylthiol, a thiocholesterol, an aliphatic chain, for example, dodecandiol or undecyl residues, a phospholipid, for example, di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

Fatty acid modified gapmer antisense oligonucleotides are described for example in Hvam et al, Molecular Therapy 25(7) July 2017.

### Chimeric Compounds

As would be appreciated by those skilled in the art, it is not necessary for all positions in a given compound to be uniformly modified and in fact, more than one of the aforementioned modifications may be incorporated in a single oligonucleotide or even at a single nucleoside within an oligonucleotide.

In certain embodiments, compounds described herein comprise oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

Certain embodiments disclosed herein provide a compound or composition comprising a modified oligonucleotide comprising: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar. In certain embodiments, at least one internucleoside linkage is a phosphorothioate linkage. In certain embodiments, and at least one cytosine is a 5-methylcytosine.

Antisense compounds of the disclosure include chimeric oligonucleotides. “Chimeric oligonucleotides” contain two or more chemically distinct regions, each made up of at least one monomer unit, that is, a nucleotide in the case of an oligonucleotide compound. These oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. By way of example, RNAse H is a cellular endonuclease which cleaves the RNA strand of an RNA:DNA duplex. Activation of RNase H, therefore, results in cleavage of the RNA target, thereby greatly enhancing the efficiency of oligonucleotide-mediated inhibition of gene expression. The cleavage of RNA:RNA hybrids can, in like fashion, be accomplished through the actions of endoribonucleases, such as RNAseL which cleaves both cellular and viral RNA. Cleavage of the RNA target can be routinely detected by gel electrophoresis and if necessary, associated nucleic acid hybridization techniques known in the art.

Chimeric antisense compounds of the disclosure may be formed as composite structures of two or more oligonucleotides, modified oligonucleotides, and/or oligonucleotide mimetics. Such compounds have also been referred to in the art as hybrids or gapmers. Representative United States patents that teach the preparation of such hybrid structures include, but are not limited to, U.S. Pat. Nos. 5,013,830, 5,149,797, 5,220,007, 5,256,775, 5,366,878, 5,403,711, 5,491,133, 5,565,350, 5,623,065, 5,652,355, 5,652,356, and 5,700,922.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighbouring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

### Exemplary Oligonucleotides

Illustrative antisense platforms known in the art include without limitation, morpholino, 1gen oligos, 2nd gen oligo's, gapmer, siRNA, LNA, BNA, or oligo mimetics like Peptide Nucleic acids. Oligonucleotides may be naked or formulated in liposomes. Oligonucleotides may be linked to a delivery means to cells or not. Oligonucleotides may use an endosome release agent or not.

Illustrative target specific siRNA to inhibit PSMD9 (Entrez Gene 5715 (human), SwissProt 000233 (human)) expression including human gene expression using RNA interference are available commercially, for example, from Cohesion Biosciences/Clinisciences (cat No. CRH3860), comprising 19-23 nucleotide siRNA synthetic oligonucleotide duplexes. Three different target specific siRNA are provided including 2′-OMe modification to provide enhanced stability and knockdown in vitro and in vivo.

PSMD9 sequences are described in publically available databases such as Genbank. A number of different variants are described by sequence. Variant 1 represents the longest transcript and encodes the longer isoform. The gene is conserved in mammalian species.

In one embodiment, the antisense compound is a second generation phosphorothioate backbone 2′-MOE-modified chimeric oligonucleotide gapmer designed to hybridize to the 3′-untranslated region of PSMD9 mRNA. In one embodiment, the oligonucleotide selectively inhibits PSMD9 expression in both primary human cells and in several human cell lines by hybridizing to RNA encoding PSMD9. In one embodiment, the oligonucleotides inhibits expression of PSMD9 in adipocytes.

In one embodiment, all uracils are 5-methyluracils (MeU). Typically, the oligonucleotide is synthesized using 2-methoxyethyl modified thymidines not 5-methyluracils.

In one embodiment, all pyrimidines are C5 methylated (i.e., U, T, C are C5 methylated).

In one embodiment, the sequence of the oligonucleotide may be named by accepted oligonucleotide nomenclature, showing each 0-0 linked phosphorothioate internucleotide linkage. In one embodiment, the PSMD9 antisense oligonucleotide has a nucleobase sequence comprising at least 8 contiguous nucleotide bases complementary to a PSMD9 polynucleotide sequence. In one embodiment, antisense oligonucleotides are complementary to at least 8 nucleotides from the 3′UTR, CDS and or 5′UTS or directed to at least one exon or one intron or a flanking region thereof.

In one embodiment, the PSMD9 inhibitor is an antisense oligonucleotide having 8 to 30 linked nucleosides having a nucleobase sequence comprising a complementary region comprising at least 8 contiguous nucleobases complementary to a target region of equal length within an exon of the PSMD9 transcript.

In one embodiment, the antisense oligonucleotide is a single stranded modified oligonucleotide. In one embodiment, the antisense oligonucleotide is chimeric (such as a RNA:DNA).

In one embodiment the antisense oligonucleotide has at least one modified internucleoside linkage, sugar or nucleobase.

Illustrative antisense oligonucleotides comprise a central gap region of 8-14 DNA nucleotides adjoined on either end with 2′-O-methoxyethyl RNA (MOE) nucleotides and phosphorothioate (PS) backbone chemistry. See Teplova et al. Nat. Struct. Biol 1999, 6:535-539; Monia et al. J. Biol. Chem. 1993, 268:14514.

In one embodiment, the internucleoside linkage is a phosphorothioate internucleoside linkage, the modified sugar is a bicyclic sugar or 2′-O-methyoxyethyl and the modified nucleobase is a 5-methylcytosine.

Depending upon the length of the antisense oligonucleotide gapmers may comprise for example a 5-10-5 design, that is, five 2′-O-methoxyethyl nucleotides at the 5′ end, 10 deoxynucleotides in the center, five 2′-O-methoxyethyl nucleotides at the 3′ end, and phosphorothioate substitution throughout. 16mer gapmers may employ a 2-12-2, 3-10-3 or 4-8-4 design etc.

In one embodiment, the PSMD9 antisense oligonucleotide comprises a gap segment consisting of linked deoxynucleosides; a 5′ wing segment consisting of linked nucleosides; and a 3′ wing segment consisting linked nucleosides; wherein the gap segment is positioned immediately adjacent to and between the 5′ wing segment and the 3′ wing segment and wherein each nucleoside of each wing segment comprises a modified sugar.

In one embodiment, PSMD9 antisense oligonucleotides are designed to preferentially affect adipose tissue.

In one embodiment a series of chimeric 20-mer phosphorothioate antisense oligonucleotides containing 2′-O-methoxyethyl groups at positions 1 to 5 and 16 to 20 targeted to murine and human PSMD9 mRNA are synthesized and purified on an automated DNA synthesizer using phosphoramidite chemistry. In one embodiment the 3′/5′ ends are locked nucleic acid or 2′O-methoxyethyl ribose.

In one embodiment the antisense oligonucleotide comprises a conjugated GalNAc. Triantennary N-acetylgalatosamine conjugated ASO. Such ASO are described for example by Prakash et al (above).

In one embodiment, the PSMD9 antisence inhibitor comprises: a gap segment consisting of 8 linked deoxynucleosides; (b) a 5′ wing segment consisting of 4 linked nucleosides; (c) a 3′ wing segment consisting 4 linked nucleosides; wherein the gap segment is positioned immediately adjacent to and between the 5′ wing segment and the 3′ wing segment, wherein each nucleoside of each wing segment comprises a 2′-O-methyoxyethyl sugar, wherein each cytosine is a 5′-methylcytosine, and wherein each internucleoside linkage is a phosphorothioate linkage. Other 16-mer gapmers will be designed in a 2-12-2 or 3-10-3 configuration as known in the art.

In one embodiment, the PSMD9 inhibitor antisense oligonucleotide is in a salt form.

In one non-limiting embodiment, the oligonucleotide may be synthesized by a multi-step process that may be divided into two distinct operations: solid-phase synthesis and downstream processing. In the first operation, the nucleotide sequence of the oligonucleotide is assembled through a computer-controlled solid-phase synthesizer. Subsequent downstream processing includes deprotection steps, preparative reversed-phase chromatographic purification, isolation and drying to yield the oligonucleotide drug substance. The chemical synthesis of the oligonucleotide utilizes phosphoramidite coupling chemistry followed by oxidative sulfurization and involves sequential coupling of activated monomers to an elongating oligomer, the 3′-terminus of which is covalently attached to the solid support.

### Detritylation (Reaction a)

Each cycle of the solid-phase synthesis commences with removal of the acid-labile 5′-O-4, 4′-dimethoxytrityl (DMT) protecting group of the 5′ terminal nucleoside of the support bound oligonucleotide. This is accomplished by treatment with an acid solution (for example dichloroacetic acid (DCA) in toluene). Following detritylation, excess reagent is removed from the support by washing with acetonitrile in preparation for the next reaction.

### Coupling (Reaction b)

Chain elongation is achieved by reaction of the 5′-hydroxyl group of the support-bound oligonucleotide with a solution of the phosphoramidite corresponding to that particular base position (e.g., for base2: MOE-MeC amidite) in the presence of an activator (e.g., 1H-tetrazole). This results in the formation of a phosphite triester linkage between the incoming nucleotide synthon and the support-bound oligonucleotide chain. After the coupling reaction, excess reagent is removed from the support by washing with acetonitrile in preparation for the next reaction.

### Sulfurization (Reaction c)

The newly formed phosphite triester linkage is converted to the corresponding [O, O, O)-trialkyl phosphorothioate triester by treatment with a solution of a sulfur transfer reagent (e.g., phenylacetyl disulfide). Following sulfurization, excess reagent is removed from the support by washing with acetonitrile in preparation for the next reaction.

### Capping (Reaction d)

A small proportion of the 5′-hydroxy groups available in any given cycle fail to extend. Coupling of these groups in any of the subsequent cycles would result in formation of process-related impurities (“DMT-on (n−1)-mers”) which are difficult to separate from the desired product. To prevent formation of these impurities and to facilitate purification, a “capping reagent” (e.g., acetic anhydride and N-methylimidazole/acetonitrile/pyridine) is introduced into the reactor vessel to give capped sequences. The resulting failure sequences (“DMT-off shortmers”) are separated from the desired product by reversed phase HPLC purification. After the capping reaction, excess reagent is removed from the support by washing with acetonitrile in preparation of the next reaction.

Reiteration of this basic four-step cycle using the appropriate protected nucleoside phosphoramidite allows assembly of the entire protected oligonucleotide sequence.

### Backbone Deprotection (Reaction e)

Following completion of the assembly portion of the process the cyanoethyl groups protecting the (O, O, O)-trialkyl phosphorothioate triester internucleotide linkages are removed by treatment with a solution of triethylamine (TEA) in acetonitrile. The reagent and acrylonitrile generated during this step are removed by washing the column with acetonitrile.

Cleavage from Support and Base Deprotection (Reaction f)

Deprotection of the exocyclic amino groups and cleavage of the crude product from the support is achieved by incubation with aqueous ammonium hydroxide (reaction f). Purification of the crude, 5′-O-DMT-protected product is accomplished by reversed phase HPLC. The reversed phase HPLC step removes DMT-off failure sequences. The elution profile is monitored by UV absorption spectroscopy. Fractions containing DMT-on oligonucleotide product are collected and analyzed.

### Acidic Deprotection (Reaction g)

Reversed phase HPLC fractions containing 5′-O-DMT-protected oligonucleotide are pooled and transferred to a precipitation tank. The products obtained from the purification of several syntheses are combined at this stage of the process. Purified DMT-on oligonucleotide is treated with acid (e.g., acetic acid) to remove the DMT group attached to the 5′ terminus. After acid exposure for the prescribed time and neutralization, the oligonucleotide drug substance is isolated and dried.

Following the final acidic deprotection step, the solution is neutralized by addition of aqueous sodium hydroxide and the oligonucleotide drug substance is precipitated from solution by adding ethanol. The precipitated material is allowed to settle at the bottom of the reaction vessel and the ethanolic supernatant decanted. The precipitated material is redissolved in purified water and the solution pH adjusted to between pH 7.2 and 7.3. The precipitation step is repeated. The precipitated material is dissolved in water and the solution filtered through a 0.45 micron filter and transferred into disposable polypropylene trays that are then loaded into a lyophilizer. The solution is cooled to −50° C. Primary drying is carried out at 25° C. for 37 hours. The temperature is increased to 30° C. and a secondary drying step performed for 5.5 hours. Following completion of the lyophilization process, the drug substance is transferred to high density polyethylene bottles and stored at −200° C.

### Target Nucleic Acid

“Targeting” an antisense compound to a particular nucleic acid can be a multistep process. The targeting process usually also includes determination of at least one target region, segment, or site within the target nucleic acid for the antisense interaction to occur such that the desired effect, for example, inhibition of expression, will result. The term “region” as used herein is defined as a portion of the target nucleic acid having at least one identifiable structure, function, or characteristic. Within regions of the target nucleic acids are segments. “Segments” are defined as smaller or sub-portions of regions within a target nucleic acid. “Sites” as used herein, means positions within the target nucleic acid.

Since the “translation initiation codon” is typically 5′-AUG (in transcribed mRNA molecules; 5′-ATG in the corresponding DNA molecule), the translation initiation codon is also referred to as the “AUG codon”, the “start codon” or the “AUG start codon”. A minority of genes have a translation initiation codon having the RNA sequence 5′-GUG, 5′-UUG, or 5′-CUG, and 5′-AUA, 5′-ACG and 5′-CUG have been shown to function in vivo. Thus, the terms “translation initiation codon” and “start codon” can encompass many codon sequences even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. The terms “start codon” and “translation initiation codon” as used herein refer to the codon or codons that are used in vivo to initiate translation of an mRNA transcribed from a gene encoding.

A “translation termination codon” also referred to a “stop codon” may have one of three RNA sequences: 5′-UAA, 5′-UAG and 5′-UGA (5′-TAA, 5′-TAG and 5′-TGA, respectively in the corresponding DNA molecule). The terms “translation termination codon” and “stop codon” as used herein refer to the codon or codons that are used in vivo to terminate translation of an mRNA transcribed from a gene encoding PSMD9 regardless of the sequence(s) of such codons.

The terms “start codon region” and “translation initiation codon region” refer to a portion of the mRNA or gene that encompasses from about 25 to about 50 contiguous nucleotides in either direction (i.e., 5′ or 3′) from the translation initiation codon. Similarly, the terms and “stop codon region” and “translation termination codon region” refer to a portion of the mRNA or gene that encompasses from about 25 to about 50 contiguous nucleotides in either direction (i.e., 5′ or 3′) from the translation termination codon. Consequently, the “start codon region” or “translation initiation codon region” and the “stop codon region” or “translation termination codon region” are all regions which may be targeted effectively with the antisense compounds of the present disclosure.

The “open reading frame” (ORF) or “coding region”, which is known in the art to refer to the region between the translation initiation codon and the translation termination codon, is also a region which may be targeted effectively. In one embodiment, the intragenic region encompassing the translation initiation or termination codon of the ORF of a gene is targeted.

Other target regions include the 5′ untranslated region (5′UTR), known in the art to refer to the portion of the mRNA in the 5′ direction from the translation initiation codon, and thus including nucleotides between the 5′ cap site and the translation initiation codon of the mRNA (or corresponding nucleotides on the gene), and the 3′ untranslated region (3′UTR), known in the art to refer to the portion of the mRNA in the 3′ direction from the translation termination codon, and thus including nucleotides between the translation termination codon and 3′ end of the mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself, as well as the first 50 nucleotides adjacent to the cap site. In one embodiment, the 5′ cap region is targeted.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA transcripts produced via the process of splicing of two (or more) mRNAs from different gene sources are known as “fusion transcripts”. In one embodiment, introns, or splice sites, that is, intron-exon junctions or exon-intron junctions, or aberrant fusion junctions due to rearrangements or deletions are preferentially targeted. Alternative RNA transcripts can be produced from the same genomic region of DNA. These alternative transcripts are generally known as “variants”.

“Pre-mRNA variants” are transcripts produced from the same genomic DNA that differ from other transcripts produced from the same genomic DNA in either their start or stop position and contain both intronic and exonic sequence. Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

Variants can be produced through the use of alternative signals to start or stop transcription, that is through use of an alternative start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. In one embodiment, the pre-mRNA or mRNA variants are targeted.

The location on the target nucleic acid to which the antisense compound hybridizes is referred to as the “target segment”. As used herein the term “target segment” is defined as at least an 8-nucleobase portion of a target region to which an antisense compound is targeted. While not wishing to be bound by theory, it is presently believed that these target segments represent portions of the target nucleic acid which are accessible for hybridization.

Once one or more target regions, segments or sites have been identified, antisense compounds are chosen which are sufficiently complementary to a target segment, that is, antisense compounds that hybridize sufficiently well and with sufficient specificity, to give the desired effect.

The target segment may also be combined with its respective complementary antisense compound to form stabilized double-stranded (duplexed) oligonucleotides. Such double stranded oligonucleotide moieties have been shown in the art to modulate target expression and regulate translation, as well as RNA processing via an antisense mechanism. Moreover, the double-stranded moieties may be subject to chemical modifications (Fire et al., 1998; Timmons and Fire, 1998; Timmons et al., 2001; Tabara et al., 1998; Montgomery et al., 1998; Tuschl et al., 1999; Elbashir et al., 2001a; Elbashir et al., 2001b). For example, such double-stranded moieties have been shown to inhibit the target by the classical hybridization of antisense strand of the duplex to the target, thereby triggering enzymatic degradation of the target (Tijsterman et al., 2002).

### Antisense Compositions

Antisense compounds of the disclosure may be admixed, encapsulated, conjugated or otherwise associated with other molecules, molecule structures or mixtures of compounds, resulting in, for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921, 5,354,844, 5,416,016, 5,459,127, 5,521,291, 5,543,158, 5,547,932, 5,583,020, 5,591,721, 4,426,330, 4,534,899, 5,013,556, 5,108,921, 5,213,804, 5,227,170, 5,264,221, 5,356,633, 5,395,619, 5,416,016, 5,417,978, 5,462,854, 5,469,854, 5,512,295, 5,527,528, 5,534,259, 5,543,152, 5,556,948, 5,580,575, and 5,595,756.

Antisense compounds of the disclosure may be administered in a pharmaceutically acceptable carrier. The term “pharmaceutically acceptable carrier” refers to molecular entities that do not produce an allergic, toxic or otherwise adverse reaction when administered to a subject, particularly a mammal, and more particularly a human. The pharmaceutically acceptable carrier may be solid or liquid. Useful examples of pharmaceutically acceptable carriers include, but are not limited to, diluents, solvents, surfactants, excipients, suspending agents, buffering agents, lubricating agents, adjuvants, vehicles, emulsifiers, absorbents, dispersion media, coatings, stabilizers, protective colloids, adhesives, thickeners, thixotropic agents, penetration agents, sequestering agents, isotonic and absorption delaying agents that do not affect the activity of the active agents of the disclosure.

In one embodiment, the pharmaceutical carrier is water for injection (WFI) and the pharmaceutical composition is adjusted to a physiologically and functionally acceptable.

In one embodiment, the salt is a sodium or potassium salt.

The oligonucleotides may contain chiral (asymmetric) centers or the molecule as a whole may be chiral. The individual stereoisomers (enantiomers and diastereoisomers) and mixtures of these are within the scope of the present disclosure.

Antisense compounds of the disclosure may be pharmaceutically acceptable salts, esters, or salts of the esters, or any other compounds which, upon administration are capable of providing (directly or indirectly) the biologically active metabolite.

The term “pharmaceutically acceptable salts” as used herein refers to physiologically and pharmaceutically acceptable salts of the antisense compounds that retain the desired biological activities of the parent compounds and do not impart undesired toxicological effects upon administration. Examples of pharmaceutically acceptable salts and their uses are further described in U.S. Pat. No. 6,287,860.

Antisense compounds of the disclosure may be prodrugs or pharmaceutically acceptable salts of the prodrugs, or other bioequivalents. The term “prodrugs” as used herein refers to therapeutic agents that are prepared in an inactive form that is converted to an active form (i.e., drug) upon administration by the action of endogenous enzymes or other chemicals and/or conditions. In particular, prodrug forms of the antisense compounds of the disclosure are prepared as SATE [(S acetyl-2-thioethyl) phosphate] derivatives according to the methods disclosed in WO 93/24510, WO 94/26764 and U.S. Pat. No. 5,770,713.

A prodrug may, for example, be converted within the body, e. g. by hydrolysis, into its active form that has medical effects. Pharmaceutical acceptable prodrugs are described in T. Higuchi and V. Stella, Prodrugs as Novel Delivery Systems, Vol. 14 of the A. C. S. Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987, which are incorporated herein by reference. Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

### Polynucleotides Encoding Peptides or Polypeptides

In one embodiment, the polynucleotide PSMD9 modulator encodes a polypeptide so that delivery of the polynucleotide leads to expression of the modulator in a suitable cell. Dominant negative inhibitors are known in the art and are contemplated herein.

In one embodiment, the polynucleotide PSMD9 modulator encodes a programmable nuclease which inhibits PSMD9 activity by inactivating or reducing expression of psmd9 gene. Programmable nucleases include RNA guided engineered nucleases derived from CRISPR-cas, ZFN, TALEN and argonaute nucleases. Such targeted nucleases are particularly useful for modulating PSMD9 in cells ex vivo.

In one embodiment, the polynucleotide is provided in an expression vector to be delivered in vivo to a subject or in vitro to a cell or tissue. Transfection methods are known in the art. A vector may be a viral vector or a non-viral vector as known in the art. For example, viral vectors include lentiviral, retroviral, adenoviral, herpes virus and adeno-associated viruses known in the art. Non-viral vectors include plasmids, transposon-modified polynucleotides (such as the MVM intron), lipoplexes, polymersomes, polyplexes, dendrimers, inorganic nanoparticles, cell penetrating peptides and combinations thereof. A new class of vectors acts by passive permeabilization of the plasma membrane. It includes peptides, streptolysin O, and cationic derivatives of polyene antibiotics. Promoters including minimal promoters and other regulatory elements which may be tissue specific.

In another embodiment, the polynucleotide PSMD9 modulator is a synthetic chemically modified RNA that encodes a dominant negative PSMD9. Typically, chemically modified mRNAs comprise (i) a 5′ synthetic cap for enhanced translation; (ii) modified nucleotides that confer RNAse resistance and an attenuated cellular interferon response, which would otherwise greatly reduce translational efficiency; and (iii) a 3′ poly-A tail. Typically, chemically modified mRNAs are synthesized in vitro from a DNA template comprising an SP6 or T7 RNA polymerase promoter-operably linked to an open reading frame encoding the dominant-negative CIS. The chemically modified mRNA synthesis reaction is carried in the presence of a mixture of modified and unmodified nucleotides. In some embodiments modified nucleotides included in the in vitro synthesis of chemically modified mRNAs are pseudo-uridine and 5-methyl-cytosine. A key step in cellular mRNA processing is the addition of a 5′ cap structure, which is a 5′-5′ triphosphate linkage between the 5′ end of the RNA and a guanosine nucleotide. The cap is methylated enzymatically at the N-7 position of the guanosine to form mature mCAP. When preparing dominant-negative PSMD9 chemically modified mRNAs, a 5′ cap is typically added prior to transfection of cells ex vivo in order to stabilize the modified mRNA and significantly enhance translation. Systems for in vitro synthesis are commercially available, as exemplified by the mRNAExpress™ mRNA Synthesis Kit (System Biosciences, Mountain View, Calif.). The general synthesis and use of such modified RNAs for in vitro and in vivo transfection are described in, e.g., WO 2011/130624, and WO/2012/138453.

### Screening

PSMD9 modulators may be identified using art recognized screening tools, such as, for example, ELISA-type assays, FRET and time resolved-FRET assays, bead based assays followed by MALDI spectrometry to name a few. Alternatively biochemical assays or cell based screens such as protein complementation, two hybrid assays are used to probe potential protein interactions.

In silico screening assays are described in the prior art for identifying potentially interacting elements and molecules from three dimensional molecule databases which can then be modified to enhance interactions. Design of peptides and analogues, derivatives and mimetics is described in the literature, see, for example Bryan et al. Peptides 2011, 32(12):2504-2510.

Further Definitions

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“2′-O-methoxyethyl” (also 2′-MOE and 2′-O(CH2)2-OCH3) refers to an O-methoxy-ethyl modification at the 2′ position of a furanosyl ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

“3′ target site” refers to the nucleotide of a target nucleic acid which is complementary to the 3′-most nucleotide of a particular compound.

“5′ target site” refers to the nucleotide of a target nucleic acid which is complementary to the 5′-most nucleotide of a particular compound.

“5-methylcytosine” means a cytosine with a methyl group attached to the 5 position.

“Antisense compound” means a compound comprising an oligonucleotide and optionally one or more additional features, such as a conjugate group or terminal group. Examples of antisense compounds include single-stranded and double-stranded compounds, such as, oligonucleotides, ribozymes, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels in the absence of the antisense compound.

“Antisense oligonucleotide” means an oligonucleotide having a nucleobase sequence that is complementary to a target nucleic acid or region or segment thereof. In certain embodiments, an antisense oligonucleotide is specifically hybridizable to a target nucleic acid or region or segment thereof.

“Bicyclic nucleoside” or “BNA” means a nucleoside comprising a bicyclic sugar moiety. “Bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“cEt” or “constrained ethyl” means a furanosyl sugar moiety comprising a bridge connecting the 4′-carbon and the 2′-carbon, wherein the bridge has the formula: 4′-CH(CH3)-O-2′.

“Chemical modification” in a compound describes the substitutions or changes through chemical reaction, of any of the units in the compound. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Double-stranded compound” means a compound comprising two oligomeric compounds that are complementary to each other and form a duplex, and wherein one of the two said oligomeric compounds comprises an oligonucleotide.

“Gapmer” means an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“Motif” means the pattern of unmodified and/or modified sugar moieties, nucleobases, and/or internucleoside linkages, in an oligonucleotide.

“Natural” or “naturally occurring” means found in nature.

“Non-bicyclic modified sugar” or “non-bicyclic modified sugar moiety” means a modified sugar moiety that comprises a modification, such as a substituent, that does not form a bridge between two atoms of the sugar to form a second ring.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A “universal base” or “universal nucleobase” is a nucleobase other than a naturally occurring nucleobase and modified nucleobase, and is capable of pairing with any nucleobase.

“Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric compound” means a compound comprising a single oligonucleotide and optionally one or more additional features, such as a conjugate group or terminal group.

“Oligonucleotide” means a polymer of linked nucleosides each of which can be modified or unmodified, independent one from another. Unless otherwise indicated, oligonucleotides consist of 8-80 linked nucleosides. “Modified oligonucleotide” means an oligonucleotide, wherein at least one sugar, nucleobase, or internucleoside linkage is modified. “Unmodified oligonucleotide” means an oligonucleotide that does not comprise any sugar, nucleobase, or internucleoside modification. “Phosphorothioate linkage” means a modified phosphate linkage in which one of the non-bridging oxygen atoms is replaced with a sulfur atom. A phosphorothioate internucleoside linkage is a modified internucleoside linkage.

“Phosphorus moiety” means a group of atoms comprising a phosphorus atom. In certain embodiments, a phosphorus moiety comprises a mono-, di-, or tri-phosphate, or phosphorothioate.

“Region” is defined as a portion of the target nucleic acid having at least one identifiable structure, function, or characteristic.

“RNAi compound” means an antisense compound that acts, at least in part, through RISC or Ago2, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.

“Segments” are defined as smaller or sub-portions of regions within a nucleic acid.

“Single-stranded” in reference to a compound means the compound has only one oligonucleotide. “Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.

“Sites,” are defined as unique nucleobase positions within a target nucleic acid.

“Specifically inhibit” a target nucleic acid means to reduce or block expression of the target nucleic acid while exhibiting fewer, minimal, or no effects on non-target nucleic acids reduction and does not necessarily indicate a total elimination of the target nucleic acid's expression.

“Sugar moiety” means an unmodified sugar moiety or a modified sugar moiety.

“Unmodified sugar moiety” or “unmodified sugar” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate. “Modified furanosyl sugar moiety” means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

“Target gene” refers to a gene encoding a target.

“Targeting” means specific hybridization of a compound to a target nucleic acid in order to induce a desired effect.

“Target nucleic acid,” “target RNA,” “target RNA transcript” and “nucleic acid target” all mean a nucleic acid capable of being targeted by compounds described herein.

“Target region” means a portion of a target nucleic acid to which one or more compounds is targeted.

The present description employs methods and material including the following:

ASOs were designed and synthesized by Ionis Pharmaceuticals. Chimeric 16-oligonucleotide phosphorothioate oligonucleotides targeted to mouse Psmd9 (eg 5′-CTCTATGGGTGCCAGC-3′) or control (5′-GGCCAATACGCCGTCA-3′) sequences were synthesized and purified as previously described (Parker et al, Nature, 2019).

ASO were tested and selected as described in the Examples of published International (PCT) Application no. PCT/AU2019/050033 published 25 Jul. 2019 as International publication no. WO 2019/140488 as disclosed therein or as incorporated herein in its entirety.

Most of the modified oligonucleotide listed in the Tables of WO 2019/140488 are targeted to either the mouse PSMD9 mRNA, designated herein as SEQ ID NO.: 10 (GENBANK Accession No. NM_026000.2) or to the mouse PSMD9 genomic sequence, designated herein as SEQ ID NO.: 11 (GENBANK Accession No. NC_000071.6 truncated from nucleotides 123225001 to 123253000). ASO are tested for their ability to down regulate PSMD9 expression in adipose tissue using methods understood in the art.

Sequences for illustrative effective mouse PSMD9 ASOs and the scrambled (control) ASO are as follows:

(SEQ ID NO: 6 (Ionis no. 998276))
D9 ASO 3: CTCTATGGGTGCCAGC
(SEQ ID NO: 7 (Ionis no. 998263))
D9 ASO 5: CTCTATCTGAGCACAC
(SEQ ID NO: 8 (Ionis no. 998164))
D9 ASO 6: GTATTTTTAGCCAGAC
(SEQ ID NO: 9 scrambled control)
ScrASO: GGCCAATACGCCGTCA.

ASO 6 (Ionis no. 998164) is also effective against SEQ ID NO: 1 encoding human PSMD9 (Table 1)—this ASO binds to residues 7734 to 7749 of SEQ ID NO: 11 as shown in Table 1.

Other PSMD9 ASO are described in Example 3 and 4 and Tables 1 to 6 of WO 2019/140488 set out below.

Antisense inhibition of mouse PSMD9 in 4T1 cells by cET gapmers—Modified oligonucleotides were designed to target a PSMD9 nucleic acid and were tested for their effect on PSMD9 RNA levels in vitro. The modified oligonucleotides were tested in a series of experiments that had similar culture conditions. The results for each experiment are presented in separate tables shown below.

The newly designed modified oligonucleotides in the tables below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides each. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines. In one embodiment, oligonucleotides target intron sequences within pre-mRNA, in one embodiment, oligonucleotides target repeat regions within pre-mRNA in the nucleus as illustrated herein.

“Start site” indicates the 5′-most nucleoside to which the gapmer is targeted in the mouse gene sequence. “Stop site” indicates the 3′-most nucleoside to which the gapmer is targeted mouse gene sequence. Most of the modified oligonucleotide listed in the Tables below are targeted to either the mouse PSMD9 mRNA, designated herein as SEQ ID NO.: 10 (GENBANK Accession No. NM_026000.2) or to the mouse PSMD9 genomic sequence, designated herein as SEQ ID NO.: 11 (GENBANK Accession No. NC_000071.6 truncated from nucleotides 123225001 to 123253000).

4T1 cells at a density of 7,000 cells per well were treated using free uptake with 7,000 nM of modified oligonucleotide. After a treatment period of approximately 48 hours, RNA was isolated from the cells and PSMD9 mRNA levels were measured by quantitative real-time RTPCR. Mouse primer probe set RTS37638 (forward sequence TGATCCGCAGAGGAGAGAA, designated herein as SEQ ID NO.: 12; reverse sequence GATCCCAGGAAACAGTCATCTC; designated herein as SEQ ID NO.: 13; probe sequence AGGACTGCTGGGCTGCAACATTAT, designated herein as SEQ ID NO.: 14) was used to measure RNA levels. PSMD9 mRNA levels were normalized to total RNA content, as measured by RIBOGREEN®. Results are presented as percent inhibition of PSMD9 relative to untreated control cells. As used herein, a value of ‘0’ indicates that treatment with the modified oligonucleotide did not inhibit PSMD9 mRNA levels. Compound numbers marked with an asterisk (*) indicate that the modified oligonucleotide is complementary to the amplicon region of the primer probe set. Additional assays may be used to measure the potency and efficacy of the modified oligonucleotides complementary to the amplicon region. Compound numbers marked with a hashtag (#) indicate that the modified oligonucleotide targets multiple sites on the nucleic acid. All start sites for the gapmer will be specified in the corresponding sub-table.

TABLE 1
Inhibition of PSMD9 RNA by 3-10-3 MOE gapmers targeting SEQ ID NO.: 1, and 2

| | SEQ ID | SEQ ID | SEQ ID | SEQ ID | | | |
|---|---|---|---|---|---|---|---|
| | NO: 10 | NO: 10 | NO: 11 | NO: 11 | | PSMD9 | SEQ |
| Compound | Start | Stop | Start | Stop | | (% Inhi- | ID |
| Number | Site | Site | Site | Site | Sequence (5′ to 3′) | bition) | NO |
| 997988 | 3 | 18 | 3192 | 3207 | GCAAGTACGGAAACAG | 0 | 15 |
| 997992 | 45 | 60 | 3234 | 3249 | GGCTACGGGTCCTCCC | 4 | 16 |
| 998000 | 143 | 158 | 3332 | 3347 | TCGGAGGACTCTGCCC | 8 | 17 |
| 998004 | 165 | 180 | 3354 | 3369 | GCTGACCGCGGCCGCA | 0 | 18 |
| 998008 | 226 | 241 | 3415 | 3430 | CGTAATTAGCCTTGAT | 3 | 19 |
| 998012 | 342 | 357 | 9680 | 9695 | GATGATGTTGTGCCTT | 0 | 20 |
| 998016 | 473 | 488 | 14656 | 14671 | AGCCTGCGGTTCATGG | 0 | 21 |
| 998020 | 529 | 544 | 14712 | 14727 | GGCTGATACTGTTCAC | 60 | 22 |
| 998028 | 608 | 623 | 16872 | 16887 | AAGTTTTGGGTGTTCA | 40 | 23 |
| 998032* | 714 | 729 | 21238 | 21253 | TGGAATCAGTCTGAGC | 82 | 24 |
| 998040 | 864 | 879 | 23398 | 23413 | CACTTAAGGGAGCCTA | 0 | 25 |
| 998044 | 898 | 913 | 23432 | 23447 | GCCCAGGCTTCGACCA | 0 | 26 |
| 998048 | 939 | 954 | 23473 | 23488 | GAGATTACATCAGGCA | 70 | 27 |
| 998052 | 976 | 991 | 23510 | 23525 | GGCACAAATCACACTT | 44 | 28 |
| 998056 | 1000 | 1015 | 23534 | 23549 | CCTAATTTGCACAAGA | 53 | 29 |
| 998060 | 1028 | 1043 | 23562 | 23577 | ATCTAGAGAATTCCCA | 8 | 30 |
| 998064 | 1067 | 1082 | 23601 | 23616 | TCATTACTCGCCAGAG | 0 | 31 |
| 998068 | 1074 | 1089 | 23608 | 23623 | CATCAAATCATTACTC | 0 | 32 |
| 998072 | 1190 | 1205 | 23724 | 23739 | ATACTAATGAGGCAGA | 16 | 33 |
| 998076 | 1210 | 1225 | 23744 | 23759 | AGTATATGCCTCTCAT | 29 | 34 |
| 998088 | 1406 | 1421 | 23940 | 23955 | TAGTAGGTTATTTATT | 15 | 35 |
| 998092 | 1432 | 1447 | 23966 | 23981 | AATATACTGACAGCAC | 13 | 36 |
| 998096 | 1451 | 1466 | 23985 | 24000 | TGGAAGATCCCACACC | 13 | 37 |
| 998100 | 1514 | 1529 | 24048 | 24063 | GGGTACTCAAGTCCTG | 0 | 38 |
| 998104 | 1643 | 1658 | 24177 | 24192 | CCCCTAGGCGGTGGGT | 0 | 39 |
| 998108 | 1717 | 1732 | 24251 | 24266 | GGTAAGGCCAGTGCGG | 28 | 40 |
| 998112 | 1763 | 1778 | 24297 | 24312 | TGGCATACACTATAAT | 22 | 41 |
| 998116 | 1816 | 1831 | 24350 | 24365 | AGCTACAAGACTGGCT | 0 | 42 |
| 998120 | 1937 | 1952 | 24471 | 24486 | ATCAACCGGACTGCGG | 12 | 43 |
| 998124 | 2001 | 2016 | 24535 | 24550 | GCTCAGCCCACGGAGG | 0 | 44 |
| 998128 | 2289 | 2304 | 24823 | 24838 | GGGTAACCTGCAAGGC | 37 | 45 |
| 998132 | 2301 | 2316 | 24835 | 24850 | CAATATCATACTGGGT | 45 | 46 |
| 998140 | N/A | N/A | 3574 | 3589 | AAGTTAATGCTTCCGA | 67 | 47 |
| 998144 | N/A | N/A | 4367 | 4382 | CGTCATCTGGCACCCA | 13 | 48 |
| 998148 | N/A | N/A | 4995 | 5010 | GCCGATGGTAGTGCAC | 30 | 49 |
| 998152 | N/A | N/A | 5900 | 5915 | TGCATACTGAGAGCCT | 7 | 50 |
| 998156 | N/A | N/A | 6554 | 6569 | AGTTACACCATCTTAC | 9 | 51 |
| 998160 | N/A | N/A | 7182 | 7197 | GGCAAGTTTGATCAGG | 55 | 52 |
| 998164 | N/A | N/A | 7734 | 7749 | GTATTTTTAGCCAGAC | 73 | 53 |
| 998168 | N/A | N/A | 8195 | 8210 | TGTTTGATGTCTGTCG | 64 | 54 |
| 998172 | N/A | N/A | 8851 | 8866 | TCCAGATTAGCCTTGG | 0 | 55 |
| 998176 | N/A | N/A | 9412 | 9427 | GTCCTTATAGCTACCC | 27 | 56 |
| 998180 | N/A | N/A | 9848 | 9863 | CGACATGCAACTCTGC | 24 | 57 |
| 998184 | N/A | N/A | 10474 | 10489 | GCTATTTGCACAGTGG | 62 | 58 |
| 998188 | N/A | N/A | 11158 | 11173 | TTATCTACAGTGCCAA | 56 | 59 |
| 998192 | N/A | N/A | 11970 | 11985 | AGCGACTAAGGACTCA | 55 | 60 |
| 998196 | N/A | N/A | 12566 | 12581 | TGAATCACCGTGGTCG | 1 | 61 |
| 998204 | N/A | N/A | 14238 | 14253 | GGCTCCTACCATCACG | 24 | 62 |
| 998208 | N/A | N/A | 15090 | 15105 | CACAGTAATGCCGCTC | 57 | 63 |
| 998212 | N/A | N/A | 15472 | 15487 | TGAATATTCACTGCCG | 62 | 64 |
| 998216 | N/A | N/A | 16296 | 16311 | GCGAATCCAGCTCTGA | 78 | 65 |
| 998220 | N/A | N/A | 17056 | 17071 | GCAAACTGTGTCATCC | 61 | 66 |
| 998224 | N/A | N/A | 17702 | 17717 | GGCTCAAGATCATCCT | 7 | 67 |
| 998228 | N/A | N/A | 18490 | 18505 | TGGATGTACAGCCTCG | 43 | 68 |
| 998232 | N/A | N/A | 19086 | 19101 | CACATTGGGACTCCCC | 18 | 69 |
| 998236 | N/A | N/A | 19981 | 19996 | AGGAATTGTATGGCCT | 21 | 70 |
| 998240 | N/A | N/A | 20852 | 20867 | GGGTGGTACAGCAGCT | 0 | 71 |
| 998244 | N/A | N/A | 21337 | 21352 | CAGCTCTATCTGAGCG | 9 | 72 |
| 998248 | N/A | N/A | 21352# | 21367# | GGGTACCAGCATCCCC | 5 | 73 |
| 998252 | N/A | N/A | 21356# | 21371# | CTATGGGTACCAGCAT | 82 | 74 |
| 998256 | N/A | N/A | 21364# | 21379# | CACACTCTCTATGGGT | 90 | 75 |
| 998260 | N/A | N/A | 21368# | 21383# | TGAGCACACTCTCTAT | 28 | 76 |
| 998264 | N/A | N/A | 21376# | 21391# | GCTCTATCTGAGCACA | 26 | 77 |
| 998268 | N/A | N/A | 21434# | 21449# | GGGTACCAGCATCCTC | 20 | 78 |
| 998272 | N/A | N/A | 21477# | 21492# | ATGGGTGCCAGCATCC | 7 | 79 |
| 998276 | N/A | N/A | 21481# | 21496# | CTCTATGGGTGCCAGC | 96 | 80 |
| 998280 | N/A | N/A | 21485# | 21500# | CACTCTCTATGGGTGC | 8 | 81 |
| 998284 | N/A | N/A | 21517 | 21532 | TGGGTGCCAGCATCCT | 20 | 82 |
| | | | 22050 | 22065 | | | |
| 998288 | N/A | N/A | 21801 | 21816 | GTACCAGCATTCCCAG | 63 | 83 |
| | | | 22334 | 22349 | | | |
| | | | 22498 | 22513 | | | |
| | | | 22621 | 22636 | | | |
| 998292 | N/A | N/A | 21805 | 21820 | ATGGGTACCAGCATTC | 54 | 84 |
| | | | 22338 | 22353 | | | |
| | | | 22502 | 22517 | | | |
| | | | 22625 | 22640 | | | |
| 998296 | N/A | N/A | 22745 | 22760 | GTTATTAACCACCAGT | 16 | 85 |

TABLE 1b
SEQ ID NO: 11 start sites for modified oligonucleotides complementary to repeat regions

| | | # of comp. sites | |
|---|---|---|---|
| Compound | SEQ | within SEQ ID | |
| Number | ID NO: | NO: 11 | SEQ ID NO: 2 start sites |
| 998248 | | 10 | 21352, 21393, 21557, 21721, 21885, 21926, 22090, 22254, 22418, |
| | | | 22541, 22664 |
| 998252 | | 21 | 21356, 21397, 21438, 21561, 21684, 21725, 21807, 21848, 21889, |
| | | | 21930, 21971, 22094, 22217, 22258, 22340, 22381, 22422, 22504, |
| | | | 22545, 22627, 22668 |
| 998256 | | 33 | 21364, 21405, 21446, 21487, 21528, 21569, 21610, 21651, 21692, |
| | | | 21733, 21774, 21815, 21856, 21897, 21938, 21979, 22020, 22061, |
| | | | 22102, 22143, 22184, 22225, 22266, 22307, 22348, 22389, 22430, |
| | | | 22471, 22512, 22553, 22594, 22635, 22676 |
| 998260 | | 33 | 21368, 21409, 21450, 21491, 21532, 21573, 21614, 21655, 21696, |
| | | | 21737, 21778, 21819, 21860, 21901, 21942, 21983, 22024, 22065, |
| | | | 22106, 22147, 22188, 22229, 22270, 22311, 22352, 22393, 22434, |
| | | | 22475, 22516, 22557, 22598, 22639, 22680 |
| 998264 | | 32 | 21376, 21417, 21458, 21499, 21540, 21581, 21622, 21663, 21704, |
| | | | 21745, 21786, 21827, 21868, 21909, 21950, 21991, 22073, 22032, |
| | | | 22114, 22155, 22196, 22237, 22278, 22319, 22360, 22401, 22442, |
| | | | 22483, 22524, 22565, 22606, 22647 |
| 998268 | | 6 | 21434, 21680, 21844, 21967, 22213, 22377 |
| 998272 | | 12 | 21477, 21518, 21600, 21641, 21764, 22010, 22051, 22133, 22174, |
| | | | 22297, 22461, 22584 |
| 998276 | | 12 | 21481, 21522, 21604, 21645, 21768, 22014, 22055, 22137, 22178, |
| | | | 22301, 22465, 22588 |
| 998280 | | 12 | 21485, 21526, 21608, 21649, 21772, 22018, 22059, 22141, 22182, |
| | | | 22305, 22469, 22592 |

TABLE 2
Inhibition of PSMD9 RNA by 3-10-3 MOE gapmers targeting SEQ ID NO.: 10, and 11

| | SEQ ID | SEQ ID | SEQ ID | SEQ ID | | | |
|---|---|---|---|---|---|---|---|
| | NO: 10 | NO: 10 | NO: 11 | NO: 11 | | PSMD9 | SEQ |
| Compound | Start | Stop | Start | Stop | | (% Inhi- | ID |
| Number | Site | Site | Site | Site | Sequence (5′ to 3′) | bition) | NO |
| 997989 | 7 | 22 | 3196 | 3211 | ACGCGCAAGTACGGAA | 12 | 86 |
| 997993 | 48 | 63 | 3237 | 3252 | TGAGGCTACGGGTCCT | 0 | 87 |
| 997997 | 96 | 111 | 3285 | 3300 | CCTCAAGCTAGAGTTC | 25 | 88 |
| 998001 | 147 | 162 | 3336 | 3351 | GGCCTCGGAGGACTCT | 7 | 89 |
| 998005 | 174 | 189 | 3363 | 3378 | CTGGATGTCGCTGACC | 23 | 90 |
| 998013 | 421 | 436 | 14604 | 14619 | TCTCTTTGTCCCGAGC | 10 | 91 |
| 998017 | 477 | 492 | 14660 | 14675 | GGCCAGCCTGCGGTTC | 63 | 92 |
| 998021 | 552 | 567 | 14735 | 14750 | CGCAATACTGGCTGGG | 34 | 93 |
| 998025 | 594 | 609 | 16858 | 16873 | CACGGAGCCGAACTCC | 2 | 94 |
| 998029 | 649 | 664 | 16913 | 16928 | CGCTATGCTGCACCAC | 19 | 95 |
| 998033* | 715 | 730 | 21239 | 21254 | TTGGAATCAGTCTGAG | 49 | 96 |
| 998041 | 866 | 881 | 23400 | 23415 | TACACTTAAGGGAGCC | 7 | 97 |
| 998045 | 910 | 925 | 23444 | 23459 | TTCCACCTCGATGCCC | 0 | 98 |
| 998049 | 942 | 957 | 23476 | 23491 | AGAGAGATTACATCAG | 44 | 99 |
| 998053 | 984 | 999 | 23518 | 23533 | CGTAGCTAGGCACAAA | 32 | 100 |
| 998057 | 1002 | 1017 | 23536 | 23551 | GGCCTAATTTGCACAA | 0 | 101 |
| 998061 | 1031 | 1046 | 23565 | 23580 | ATAATCTAGAGAATTC | 11 | 102 |
| 998065 | 1070 | 1085 | 23604 | 23619 | AAATCATTACTCGCCA | 38 | 103 |
| 998069 | 1102 | 1117 | 23636 | 23651 | ACTGAGTCCGTCTCCA | 64 | 104 |
| 998077 | 1211 | 1226 | 23745 | 23760 | CAGTATATGCCTCTCA | 0 | 105 |
| 998085 | 1327 | 1342 | 23861 | 23876 | GGGACTTGAGATGACA | 0 | 106 |
| 998089 | 1410 | 1425 | 23944 | 23959 | CACTTAGTAGGTTATT | 20 | 107 |
| 998093 | 1434 | 1449 | 23968 | 23983 | TGAATATACTGACAGC | 46 | 108 |
| 998097 | 1452 | 1467 | 23986 | 24001 | GTGGAAGATCCCACAC | 0 | 109 |
| 998101 | 1628 | 1643 | 24162 | 24177 | TGATACTGCAGTTGGA | 18 | 110 |
| 998105 | 1679 | 1694 | 24213 | 24228 | CTGAACTTGTGAGATC | 36 | 111 |
| 998109 | 1724 | 1739 | 24258 | 24273 | TCCCAAGGGTAAGGCC | 0 | 112 |
| 998113 | 1778 | 1793 | 24312 | 24327 | TGTAACAAGGTTTGGT | 4 | 113 |
| 998117 | 1903 | 1918 | 24437 | 24452 | TCGCAGGACTTCCTTC | 0 | 114 |
| 998121 | 1944 | 1959 | 24478 | 24493 | CCCAAGAATCAACCGG | 0 | 115 |
| 998125 | 2045 | 2060 | 24579 | 24594 | CATCAGGCTCTCAAAG | 16 | 116 |
| 998129 | 2295 | 2310 | 24829 | 24844 | CATACTGGGTAACCTG | 44 | 117 |
| 998133 | 2302 | 2317 | 24836 | 24851 | CCAATATCATACTGGG | 0 | 118 |
| 998137 | 2359 | 2374 | 24893 | 24908 | TTTTACTGTAGAAGTA | 0 | 119 |
| 998141 | N/A | N/A | 3674 | 3689 | GCGATTCCCGCACTCA | 0 | 120 |
| 998145 | N/A | N/A | 4552 | 4567 | TATGATGGCCAGTGCC | 0 | 121 |
| 998149 | N/A | N/A | 5291 | 5306 | GGTCTCTGCGGTATGC | 71 | 122 |
| 998153 | N/A | N/A | 6088 | 6103 | CTATATCCCAGACACC | 6 | 123 |
| 998157 | N/A | N/A | 6661 | 6676 | GATATATTTGCAACAA | 74 | 124 |
| 998161 | N/A | N/A | 7423 | 7438 | CACTTATCTGTTAGCT | 53 | 125 |
| 998165 | N/A | N/A | 7913 | 7928 | TAATATGGGAGCCTTC | 0 | 126 |
| 998169 | N/A | N/A | 8360 | 8375 | TGCTTTAGGGCCAGCT | 22 | 127 |
| 998173 | N/A | N/A | 9005 | 9020 | ATAGGATGTAGCTCGG | 58 | 128 |
| 998177 | N/A | N/A | 9447 | 9462 | TGATGTCTTTAGCACA | 75 | 129 |
| | | | 9466 | 9481 | | | |
| 998181 | N/A | N/A | 9861 | 9876 | ATAATAAAGCCATCGA | 43 | 130 |
| 998185 | N/A | N/A | 10624 | 10639 | ACCAATGGCACACTCA | 37 | 131 |
| 998189 | N/A | N/A | 11161 | 11176 | TGATTATCTACAGTGC | 57 | 132 |
| 998193 | N/A | N/A | 12181 | 12196 | GGCTTACAGTAGAGTC | 5 | 133 |
| 998197 | N/A | N/A | 12776 | 12791 | ATAATATTGAATCAGG | 41 | 134 |
| 998201 | N/A | N/A | 13668 | 13683 | TGCAACTATGCCCTGA | 0 | 135 |
| 998205 | N/A | N/A | 14493 | 14508 | GCTAGCGCGGGACACA | 37 | 136 |
| 998209 | N/A | N/A | 15233 | 15248 | AAAATTACTGGTGCTC | 32 | 137 |
| 998213 | N/A | N/A | 15553 | 15568 | GTCACACACGGAGAGC | 23 | 138 |
| 998217 | N/A | N/A | 16642 | 16657 | GGAGTAGGCAGGTGCC | 48 | 139 |
| 998221 | N/A | N/A | 17226 | 17241 | GACAGATACCCAGCGC | 48 | 140 |
| 998225 | N/A | N/A | 17802 | 17817 | ACCTATATCCACGGGC | 22 | 141 |
| 998229 | N/A | N/A | 18598 | 18613 | TGAGATGCGACCCCCT | 21 | 142 |
| 998233 | N/A | N/A | 19372 | 19387 | CAAGATTGCTTGCGCT | 37 | 143 |
| 998237 | N/A | N/A | 20192 | 20207 | TTCTTACTGAGACACA | 59 | 144 |
| 998241 | N/A | N/A | 20988 | 21003 | TCCTTAAGTTCCGGCA | 66 | 145 |
| 998249 | N/A | N/A | 21353# | 21368# | TGGGTACCAGCATCCC | 0 | 146 |
| 998253 | N/A | N/A | 21361# | 21376# | ACTCTCTATGGGTACC | 86 | 147 |
| 998261 | N/A | N/A | 21373# | 21388# | CTATCTGAGCACACTC | 67 | 148 |
| 998265 | N/A | N/A | 21377# | 21392# | AGCTCTATCTGAGCAC | 5 | 149 |
| 998269 | N/A | N/A | 21435# | 21450# | TGGGTACCAGCATCCT | 19 | 150 |
| 998273 | N/A | N/A | 21478# | 21493# | TATGGGTGCCAGCATC | 47 | 151 |
| 998277 | N/A | N/A | 21482# | 21497# | TCTCTATGGGTGCCAG | 78 | 152 |
| 998281 | N/A | N/A | 21486# | 21501# | ACACTCTCTATGGGTG | 0 | 153 |
| 998285 | N/A | N/A | 21791 | 21806 | TCCCAGCTCTATCTGA | 30 | 154 |
| | | | 22324 | 22339 | | | |
| | | | 22488 | 22503 | | | |
| | | | 22611 | 22626 | | | |
| 998289 | N/A | N/A | 21802 | 21817 | GGTACCAGCATTCCCA | 40 | 155 |
| | | | 22335 | 22350 | | | |
| | | | 22499 | 22514 | | | |
| | | | 22622 | 22637 | | | |
| 998293 | N/A | N/A | 21806 | 21821 | TATGGGTACCAGCATT | 51 | 156 |
| | | | 22339 | 22354 | | | |
| | | | 22503 | 22518 | | | |
| | | | 22626 | 22641 | | | |
| 998297 | N/A | N/A | 22812 | 22827 | AGGGATTGAGAAGTGA | 26 | 157 |

TABLE 2b
SEQ ID NO: 11 start sites for modified oligonucleotides complementary to repeat regions

| | | # of comp. sites | |
|---|---|---|---|
| Compound | SEQ | within SEQ ID | |
| Number | ID NO: | NO: 11 | SEQ ID NO: 11 start sites |
| 998249 | | 11 | 21353, 21394, 21558, 21722, 21886, 21927, 22091, 22255, 22419, |
| | | | 22542, 22665 |
| 998253 | | 21 | 21361, 21402, 21443, 21566, 21689, 21730, 21812, 21853, 21894, |
| | | | 21935, 21976, 22099, 22222, 22263, 22345, 22386, 22427, 22509, |
| | | | 22550, 22632, 22673 |
| 998261 | | 33 | 21373, 21414, 21455, 21496, 21537, 21578, 21619, 21660, 21701, |
| | | | 21742, 21783, 21824, 21865, 21906, 21947, 21988, 22029, 22070, |
| | | | 22111, 22152, 22193, 22234, 22275, 22316, 22357, 22398, 22439, |
| | | | 22480, 22521, 22562, 22603, 22644, 22685 |
| 998265 | | 32 | 21377, 21418, 21459, 21500, 21541, 21582, 21623, 21664, 21705, |
| | | | 21746, 21787, 21828, 21869, 21910, 21951, 21992, 22033, 22074, |
| | | | 22115, 22156, 22197, 22238, 22279, 22320, 22361, 22402, 22443, |
| | | | 22484, 22525, 22566, 22607, 22648 |
| 998269 | | 6 | 21435, 21681, 21845, 21968, 22214, 22378 |
| 998273 | | 12 | 21478, 21519, 21601, 21642, 21765, 22011, 22052, 22134, 22175, |
| | | | 22298, 22462, 22585 |
| 998277 | | 12 | 21482, 21523, 21605, 21646, 21769, 22015, 22138, 22056, 22179, |
| | | | 22302, 22466, 22589 |
| 998281 | | 12 | 21486, 21527, 21609, 21650, 21773, 22019, 22060, 22142, 22183, |
| | | | 22306, 22470, 22593 |

TABLE 3
Inhibition of PSMD9 RNA by 3-10-3 MOE gapmers targeting SEQ ID NO.: 10, and 11

| | SEQ ID | SEQ ID | SEQ ID | SEQ ID | | | |
|---|---|---|---|---|---|---|---|
| | NO: 10 | NO: 10 | NO: 11 | NO: 11 | | PSMD9 | SEQ |
| Compound | Start | Stop | Start | Stop | | (% Inhi- | ID |
| Number | Site | Site | Site | Site | Sequence (5′ to 3′) | bition) | NO |
| 997990 | 12 | 27 | 3201 | 3216 | AGCCAACGCGCAAGTA | 16 | 158 |
| 997994 | 51 | 66 | 3240 | 3255 | GGCTGAGGCTACGGGT | 0 | 159 |
| 997998 | 110 | 125 | 3299 | 3314 | CCCGACATCGCGGACC | 4 | 160 |
| 998002 | 153 | 168 | 3342 | 3357 | CGCACGGGCCTCGGAG | 14 | 161 |
| 998006 | 186 | 201 | 3375 | 3390 | TCGCATCAGATCCTGG | 0 | 162 |
| 998010 | 313 | 328 | 9651 | 9666 | GGTACAAGTCCACATC | 23 | 163 |
| 998014 | 433 | 448 | 14616 | 14631 | CCCGAGCCTGCTTCTC | 0 | 164 |
| 998018 | 526 | 541 | 14709 | 14724 | TGATACTGTTCACTCT | 0 | 165 |
| 998022 | 553 | 568 | 14736 | 14751 | CCGCAATACTGGCTGG | 15 | 166 |
| 998026 | 597 | 612 | 16861 | 16876 | GTTCACGGAGCCGAAC | 11 | 167 |
| 998030* | 675 | 690 | 21199 | 21214 | CACCGTCACATTCAGG | 0 | 168 |
| 998034* | 725 | 740 | 21249 | 21264 | GCCCAGCGGGTTGGAA | 85 | 169 |
| 998038 | 849 | 864 | 23383 | 23398 | AGAGAACGAGGAAACG | 0 | 170 |
| 998042 | 869 | 884 | 23403 | 23418 | CCTTACACTTAAGGGA | 0 | 171 |
| 998046 | 936 | 951 | 23470 | 23485 | ATTACATCAGGCAGCC | 21 | 172 |
| 998050 | 960 | 975 | 23494 | 23509 | TTAATAATGCCTCAAC | 24 | 173 |
| 998054 | 998 | 1013 | 23532 | 23547 | TAATTTGCACAAGACG | 28 | 174 |
| 998058 | 1008 | 1023 | 23542 | 23557 | GGCTATGGCCTAATTT | 0 | 175 |
| 998066 | 1072 | 1087 | 23606 | 23621 | TCAAATCATTACTCGC | 55 | 176 |
| 998070 | 1105 | 1120 | 23639 | 23654 | CACACTGAGTCCGTCT | 49 | 177 |
| 998074 | 1193 | 1208 | 23727 | 23742 | CCAATACTAATGAGGC | 0 | 178 |
| 998078 | 1212 | 1227 | 23746 | 23761 | TCAGTATATGCCTCTC | 31 | 179 |
| 998082 | 1288 | 1303 | 23822 | 23837 | GCATGTACGAAATTCT | 71 | 180 |
| 998086 | 1328 | 1343 | 23862 | 23877 | AGGGACTTGAGATGAC | 27 | 181 |
| 998090 | 1412 | 1427 | 23946 | 23961 | GGCACTTAGTAGGTTA | 33 | 182 |
| 998094 | 1435 | 1450 | 23969 | 23984 | ATGAATATACTGACAG | 0 | 183 |
| 998098 | 1457 | 1472 | 23991 | 24006 | CTCCAGTGGAAGATCC | 0 | 184 |
| 998102 | 1630 | 1645 | 24164 | 24179 | GGTGATACTGCAGTTG | 33 | 185 |
| 998106 | 1706 | 1721 | 24240 | 24255 | TGCGGGTACACTGAGC | 0 | 186 |
| 998110 | 1729 | 1744 | 24263 | 24278 | CAAGATCCCAAGGGTA | 16 | 187 |
| 998114 | 1779 | 1794 | 24313 | 24328 | CTGTAACAAGGTTTGG | 43 | 188 |
| 998118 | 1907 | 1922 | 24441 | 24456 | TGCTTCGCAGGACTTC | 16 | 189 |
| 998122 | 1992 | 2007 | 24526 | 24541 | ACGGAGGGACACTTGC | 9 | 190 |
| 998126 | 2055 | 2070 | 24589 | 24604 | TCAGAGGATGCATCAG | 52 | 191 |
| 998130 | 2297 | 2312 | 24831 | 24846 | ATCATACTGGGTAACC | 28 | 192 |
| 998134 | 2309 | 2324 | 24843 | 24858 | GAGGAAGCCAATATCA | 22 | 193 |
| 998138 | 2381 | 2396 | 24915 | 24930 | AATCAGGCCCATCTGC | 46 | 194 |
| 998142 | N/A | N/A | 3957 | 3972 | GCAAGAATAACCCTCA | 0 | 195 |
| 998146 | N/A | N/A | 4847 | 4862 | AGCTTTACCAAGCCGG | 0 | 196 |
| 998150 | N/A | N/A | 5539 | 5554 | GGTTTCTAATAGGTTT | 91 | 197 |
| 998154 | N/A | N/A | 6291 | 6306 | GTTACCACGCATGTGT | 11 | 198 |
| 998158 | N/A | N/A | 6910 | 6925 | AGCATTTCCGGGCTGG | 22 | 199 |
| 998162 | N/A | N/A | 7445 | 7460 | ACTGTATGGGTTGACT | 12 | 200 |
| 998170 | N/A | N/A | 8469 | 8484 | CTTTATACTTAGCCTC | 51 | 201 |
| 998174 | N/A | N/A | 9237 | 9252 | CCATATGCACTCCTCA | 33 | 202 |
| 998178 | N/A | N/A | 9448 | 9463 | GTGATGTCTTTAGCAC | 25 | 203 |
| | | | 9467 | 9482 | | | |
| 998182 | N/A | N/A | 10162 | 10177 | TACTTTTGTATGCAGC | 64 | 204 |
| 998186 | N/A | N/A | 10732 | 10747 | TCTAACAGGTACTTCA | 17 | 205 |
| 998190 | N/A | N/A | 11300 | 11315 | TCTTACTCTGCACCCT | 25 | 206 |
| 998194 | N/A | N/A | 12299 | 12314 | GGTCATCTAGCCTGCC | 27 | 207 |
| 998198 | N/A | N/A | 12916 | 12931 | CCTACTACTGGGCTCT | 35 | 208 |
| 998202 | N/A | N/A | 13780 | 13795 | AATATAATCACATCGG | 59 | 209 |
| 998206 | N/A | N/A | 14761 | 14776 | GGATTTGGGAGAGCCA | 20 | 210 |
| 998210 | N/A | N/A | 15345 | 15360 | CTTCATCTGTGACCCG | 84 | 211 |
| 998214 | N/A | N/A | 15773 | 15788 | TCCGAATTCAGAATCC | 29 | 212 |
| 998218 | N/A | N/A | 16763 | 16778 | GGTCATTTGTACCGCT | 35 | 213 |
| 998222 | N/A | N/A | 17365 | 17380 | GTGTAAAAGACTCAGC | 45 | 214 |
| 998226 | N/A | N/A | 17907 | 17922 | CTACTATCCATTTGGG | 10 | 215 |
| 998230 | N/A | N/A | 18809 | 18824 | TGAGGGACCGCTAACA | 0 | 216 |
| 998234 | N/A | N/A | 19515 | 19530 | CAGAAATTGTTGTTGC | 0 | 217 |
| 998238 | N/A | N/A | 20611 | 20626 | CTTACTCCGAGGGTCA | 60 | 218 |
| 998242 | N/A | N/A | 21333 | 21348 | TCTATCTGAGCGCACT | 45 | 219 |
| 998246 | N/A | N/A | 21339# | 21354# | CCCAGCTCTATCTGAG | 58 | 220 |
| 998250 | N/A | N/A | 21354# | 21369# | ATGGGTACCAGCATCC | 70 | 221 |
| 998254 | N/A | N/A | 21362# | 21377# | CACTCTCTATGGGTAC | 68 | 222 |
| 998258 | N/A | N/A | 21366# | 21381# | AGCACACTCTCTATGG | 96 | 223 |
| 998262 | N/A | N/A | 21374# | 21389# | TCTATCTGAGCACACT | 59 | 224 |
| 998270 | N/A | N/A | 21475# | 21490# | GGGTGCCAGCATCCCC | 10 | 225 |
| 998278 | N/A | N/A | 21483# | 21498# | CTCTCTATGGGTGCCA | 75 | 226 |
| 998286 | N/A | N/A | 21792 | 21807 | TTCCCAGCTCTATCTG | 49 | 227 |
| | | | 22325 | 22340 | | | |
| | | | 22489 | 22504 | | | |
| | | | 22612 | 22627 | | | |
| 998290 | N/A | N/A | 21803 | 21818 | GGGTACCAGCATTCCC | 0 | 228 |
| | | | 22336 | 22351 | | | |
| | | | 22500 | 22515 | | | |
| | | | 22623 | 22638 | | | |
| 998294 | N/A | N/A | 22687 | 22702 | TTCTATCTGAGCACAC | 69 | 229 |
| 998298 | N/A | N/A | 22973 | 22988 | TGTATATAAGAGAGTC | 56 | 230 |

TABLE 3b
SEQ ID NO: 11 start sites for modified oligonucleotides complementary to repeat regions

| | | # of comp. sites | |
|---|---|---|---|
| Compound | SEQ | within SEQ ID | |
| Number | ID NO: | NO: 11 | SEQ ID NO: 2 start sites |
| 998246 | | 25 | 21339, 21380, 21462, 21544, 21585, 21626, 21708, 21749, 21790, |
| | | | 21872, 21913, 21995, 22077, 22118, 22159, 22241, 22282, 22323, |
| | | | 22405, 22446, 22487, 22528, 22569, 22610, 22651 |
| 998250 | | 17 | 21354, 21395, 21436, 21559, 21682, 21723, 21846, 21887, 21928, |
| | | | 21969, 22092, 22215, 22256, 22379, 22420, 22543, 22666 |
| 998254 | | 21 | 21362, 21403, 21444, 21567, 21690, 21731, 21813, 21854, 21895, |
| | | | 21936, 21977, 22100, 22223, 22264, 22346, 22387, 22428, 22510, |
| | | | 22551, 22633, 22674 |
| 998258 | | 33 | 21366, 21407, 21448, 21489, 21530, 21571, 21612, 21653, 21694, |
| | | | 21735, 21776, 21817, 21899, 21858, 21940, 21981, 22022, 22063, |
| | | | 22104, 22145, 22186, 22227, 22268, 22309, 22350, 22391, 22432, |
| | | | 22473, 22514, 22555, 22596, 22637, 22678 |
| 998262 | | 33 | 21374, 21415, 21456, 21497, 21538, 21579, 21620, 21661, 21702, |
| | | | 21743, 21825, 21784, 21866, 21907, 21948, 21989, 22030, 22071, |
| | | | 22112, 22153, 22194, 22235, 22276, 22317, 22358, 22399, 22440, |
| | | | 22481, 22522, 22563, 22604, 22645, 22686, |
| 998270 | | 10 | 21475, 21598, 21639, 21762, 22008, 22131, 22172, 22295, 22459, |
| | | | 22582 |
| 998278 | | 12 | 21483, 21524, 21606, 21647, 21770, 22016, 22057, 22139, 22180, |
| | | | 22303, 22467, 22590 |

TABLE 4
Inhibition of PSMD9 RNA by 3-10-3 MOE gapmers targeting SEQ ID NO.: 10, and 11

| | SEQ ID | SEQ ID | SEQ ID | SEQ ID | | | |
|---|---|---|---|---|---|---|---|
| | NO: 10 | NO: 10 | NO: 11 | NO: 11 | | PSMD9 | SEQ |
| Compound | Start | Stop | Start | Stop | | (% Inhi- | ID |
| Number | Site | Site | Site | Site | Sequence (5′ to 3′) | bition) | NO |
| 997991 | 16 | 31 | 3205 | 3220 | GCTCAGCCAACGCGCA | 10 | 231 |
| 997995 | 79 | 94 | 3268 | 3283 | GGGTTTCCCGGCTACG | 14 | 232 |
| 997999 | 115 | 130 | 3304 | 3319 | CTTCACCCGACATCGC | 22 | 233 |
| 998003 | 156 | 171 | 3345 | 3360 | GGCCGCACGGGCCTCG | 0 | 234 |
| 998007 | 223 | 238 | 3412 | 3427 | AATTAGCCTTGATCTC | 29 | 235 |
| 998011 | 321 | 336 | 9659 | 9674 | TCGGACCTGGTACAAG | 24 | 236 |
| 998015 | 445 | 460 | 14628 | 14643 | CTTCAGCCATGTCCCG | 0 | 237 |
| 998019 | 527 | 542 | 14710 | 14725 | CTGATACTGTTCACTC | 35 | 238 |
| 998023 | 571 | 586 | 16835 | 16850 | CGTCATCCACTTGCAG | 47 | 239 |
| 998027 | 600 | 615 | 16864 | 16879 | GGTGTTCACGGAGCCG | 4 | 240 |
| 998031* | 683 | 698 | 21207 | 21222 | CTGCGGATCACCGTCA | 59 | 241 |
| 998039 | 853 | 868 | 23387 | 23402 | GCCTAGAGAACGAGGA | 20 | 242 |
| 998043 | 870 | 885 | 23404 | 23419 | TCCTTACACTTAAGGG | 0 | 243 |
| 998047 | 937 | 952 | 23471 | 23486 | GATTACATCAGGCAGC | 53 | 244 |
| 998055 | 999 | 1014 | 23533 | 23548 | CTAATTTGCACAAGAC | 9 | 245 |
| 998059 | 1013 | 1028 | 23547 | 23562 | AGACAGGCTATGGCCT | 12 | 246 |
| 998063 | 1059 | 1074 | 23593 | 23608 | CGCCAGAGTCATCCCC | 0 | 247 |
| 998067 | 1073 | 1088 | 23607 | 23622 | ATCAAATCATTACTCG | 48 | 248 |
| 998071 | 1108 | 1123 | 23642 | 23657 | TTACACACTGAGTCCG | 64 | 249 |
| 998075 | 1209 | 1224 | 23743 | 23758 | GTATATGCCTCTCATC | 0 | 250 |
| 998079 | 1244 | 1259 | 23778 | 23793 | AATACATATTCCTCAG | 48 | 251 |
| 998083 | 1289 | 1304 | 23823 | 23838 | TGCATGTACGAAATTC | 61 | 252 |
| 998087 | 1355 | 1370 | 23889 | 23904 | GGAAGTGGGTACGAGG | 56 | 253 |
| 998091 | 1418 | 1433 | 23952 | 23967 | ACAAATGGCACTTAGT | 21 | 254 |
| 998095 | 1439 | 1454 | 23973 | 23988 | CACCATGAATATACTG | 28 | 255 |
| 998099 | 1476 | 1491 | 24010 | 24025 | TGGAAGGTTGACCACA | 18 | 256 |
| 998103 | 1631 | 1646 | 24165 | 24180 | GGGTGATACTGCAGTT | 0 | 257 |
| 998107 | 1712 | 1727 | 24246 | 24261 | GGCCAGTGCGGGTACA | 0 | 258 |
| 998111 | 1755 | 1770 | 24289 | 24304 | ACTATAATACCAGGAG | 30 | 259 |
| 998115 | 1784 | 1799 | 24318 | 24333 | CTAACCTGTAACAAGG | 18 | 260 |
| 998119 | 1932 | 1947 | 24466 | 24481 | CCGGACTGCGGCCCAG | 25 | 261 |
| 998123 | 1996 | 2011 | 24530 | 24545 | GCCCACGGAGGGACAC | 0 | 262 |
| 998127 | 2082 | 2097 | 24616 | 24631 | GGCTACGGTGACTCCA | 12 | 263 |
| 998131 | 2298 | 2313 | 24832 | 24847 | TATCATACTGGGTAAC | 15 | 264 |
| 998135 | 2327 | 2342 | 24861 | 24876 | TTCCAGTGGGTTACTG | 0 | 265 |
| 998143 | N/A | N/A | 4156 | 4171 | GCTTAATCTGGCTCCA | 7 | 266 |
| 998147 | N/A | N/A | 4853 | 4868 | GTTTTAAGCTTTACCA | 49 | 267 |
| 998155 | N/A | N/A | 6406 | 6421 | GGCCTTTAAGAGTTCC | 0 | 268 |
| 998159 | N/A | N/A | 7032 | 7047 | CACAATTCCACGCTAC | 8 | 269 |
| 998163 | N/A | N/A | 7610 | 7625 | AGTACTGGGAGATAGC | 0 | 270 |
| 998171 | N/A | N/A | 8639 | 8654 | ACCAAGATTCCTCCCA | 20 | 271 |
| 998175 | N/A | N/A | 9238 | 9253 | TCCATATGCACTCCTC | 32 | 272 |
| 998179 | N/A | N/A | 9449 | 9464 | AGTGATGTCTTTAGCA | 81 | 273 |
| | | | 9468 | 9483 | | | |
| 998183 | N/A | N/A | 10473 | 10488 | CTATTTGCACAGTGGG | 50 | 274 |
| 998187 | N/A | N/A | 10902 | 10917 | CAAAGGATACACCACC | 18 | 275 |
| 998191 | N/A | N/A | 11706 | 11721 | TGGTACAGTAAGCTCT | 36 | 276 |
| 998195 | N/A | N/A | 12455 | 12470 | CCTTATTCAACCCAGG | 1 | 277 |
| 998199 | N/A | N/A | 13038 | 13053 | TGTTTAGGGTTAGCCT | 9 | 278 |
| 998203 | N/A | N/A | 13905 | 13920 | TGCTTATTAGGTGCTA | 23 | 279 |
| 998207 | N/A | N/A | 14929 | 14944 | ACCATAGGTCTCTCCC | 53 | 280 |
| 998211 | N/A | N/A | 15451 | 15466 | CGTATAATAGCCCCAA | 62 | 281 |
| 998215 | N/A | N/A | 15954 | 15969 | TTGTATGTCAGTTGCC | 86 | 282 |
| 998219 | N/A | N/A | 16927 | 16942 | CCGACTTACCCCCTCG | 38 | 283 |
| 998223 | N/A | N/A | 17486 | 17501 | CCCAATAACAGCTGCA | 0 | 284 |
| 998227 | N/A | N/A | 18076 | 18091 | CTCTATAGCAAGGTGT | 46 | 285 |
| 998231 | N/A | N/A | 18916 | 18931 | CGTGGCAGCGCACTGT | 0 | 286 |
| 998235 | N/A | N/A | 19932 | 19947 | TCAATACTCATGTTGT | 74 | 287 |
| 998239 | N/A | N/A | 20727 | 20742 | CCAATCAACAATCTGG | 16 | 288 |
| 998243 | N/A | N/A | 21336 | 21351 | AGCTCTATCTGAGCGC | 24 | 289 |
| 998247 | N/A | N/A | 21351# | 21366# | GGTACCAGCATCCCCA | 55 | 290 |
| 998251 | N/A | N/A | 21355# | 21370# | TATGGGTACCAGCATC | 64 | 291 |
| 998255 | N/A | N/A | 21363# | 21378# | ACACTCTCTATGGGTA | 74 | 292 |
| 998259 | N/A | N/A | 21367# | 21382# | GAGCACACTCTCTATG | 58 | 293 |
| 998263 | N/A | N/A | 21375# | 21390# | CTCTATCTGAGCACAC | 85 | 294 |
| 998267 | N/A | N/A | 21420# | 21435# | TCAGCTCTATCTGAGC | 19 | 295 |
| 998271 | N/A | N/A | 21476# | 21491# | TGGGTGCCAGCATCCC | 0 | 296 |
| 998275 | N/A | N/A | 21480# | 21495# | TCTATGGGTGCCAGCA | 95 | 297 |
| 998279 | N/A | N/A | 21484# | 21499# | ACTCTCTATGGGTGCC | 79 | 298 |
| 998283 | N/A | N/A | 21516 | 21531 | GGGTGCCAGCATCCTC | 12 | 299 |
| | | | 22049 | 22064 | | | |
| 998287 | N/A | N/A | 21794 | 21809 | CATTCCCAGCTCTATC | 27 | 300 |
| | | | 22327 | 22342 | | | |
| | | | 22491 | 22506 | | | |
| | | | 22614 | 22629 | | | |
| 998291 | N/A | N/A | 21804 | 21819 | TGGGTACCAGCATTCC | 72 | 301 |
| | | | 22337 | 22352 | | | |
| | | | 22501 | 22516 | | | |
| | | | 22624 | 22639 | | | |
| 998295 | N/A | N/A | 22688 | 22703 | GTTCTATCTGAGCACA | 36 | 302 |
| 998299 | N/A | N/A | 23218 | 23233 | GTGAACACTTCTTCTC | 46 | 303 |

TABLE 4b
SEQ ID NO: 11 start sites for modified oligonucleotides complementary to repeat regions

| | | # of comp. sites | |
|---|---|---|---|
| Compound | SEQ | within SEQ ID | |
| Number | ID NO: | NO: 11 | SEQ ID NO: 2 start sites |
| 998247 | | 11 | 21351, 21392, 21556, 21720, 21884, 21925, 22089, 22253, 22417, |
| | | | 22540, 22663 |
| 998251 | | 17 | 21355, 21396, 21437, 21560, 21683, 21724, 21847, 21888, 21929, |
| | | | 21970, 22093, 22216, 22257, 22421, 22380, 22544, 22667 |
| 998255 | | 21 | 998255, 998255, 998255, 998255, 998255, 998255, 998255, |
| | | | 998255, 998255, 998255, 998255, 998255, 998255, 998255, |
| | | | 998255, 998255, 998255, 998255, 998255, 998255, 998255 |
| 998259 | | 33 | 21367, 21408, 21449, 21490, 21531, 21572, 21613, 21654, 21695, |
| | | | 21736, 21777, 21818, 21859, 21900, 21941, 21982, 22023, 22064, |
| | | | 22105, 22146, 22187, 22228, 22269, 22310, 22351, 22392, 22433, |
| | | | 22474, 22515, 22556, 22597, 22638, 22679 |
| 998263 | | 32 | 21375, 21416, 21457, 21498, 21539, 21580, 21621, 21662, 21703, |
| | | | 21744, 21785, 21826, 21867, 21908, 21949, 21990, 22031, 22072, |
| | | | 22113, 22154, 22195, 22236, 22277, 22318, 22359, 22400, 22482, |
| | | | 22441, 22523, 22564, 22605, 22646 |
| 998267 | | 8 | 21420, 21502, 21666, 21830, 21953, 22035, 22199, 22363 |
| 998271 | | 10 | 21476, 21599, 21640, 21763, 22009, 22132, 22173, 22296, 22460, |
| | | | 22583 |
| 998275 | | 12 | 21480, 21521, 21603, 21644, 21767, 22013, 22054, 22136, 22177, |
| | | | 22300, 22464, 22587 |
| 998279 | | 12 | 21484, 21525, 21607, 21648, 21771, 22017, 22058, 22140, 22181, |
| | | | 22304, 22468, 22591 |

### Dose-Dependent Inhibition of Mouse PSMD9 in 4T1 Cells by cET Gapmers

Modified oligonucleotides described in the studies above exhibiting significant in vitro inhibition of PSMD9 mRNA were selected and tested at various doses in 4T1 cells.

4T1 cells plated at a density of 7,000 cells per well were treated using free uptake with modified oligonucleotides diluted to different concentrations as specified in the tables below. After a treatment period of approximately 48 hours, PSMD9 mRNA levels were measured as previously described using the mouse PSMD9 primer-probe set RTS37638. PSMD9 mRNA levels were normalized to total RNA content, as measured by RIBOGREEN®. Results are presented in the tables below as percent inhibition of PSMD9, relative to untreated control cells. As used herein, a value of ‘0’ indicates that treatment with the modified oligonucleotide did not inhibit PSMD9 mRNA levels.

The half maximal inhibitory concentration (IC₅₀) of each modified oligonucleotide is also presented. IC₅₀ was calculated using a linear regression on a log/linear plot of the data in excel. In some cases, precise IC₅₀ could not be reliably calculated as the knockdown at the lowest dose tested led to inhibition greater than 50%. In such cases, IC50s are marked as NC (Not Calculated).

TABLE 5
Multi-dose assay of modified oligonucleotides in 4T1 cells

| | % Inhibition RTS37638 | | | | IC50 |
|---|---|---|---|---|---|
| ION No. | 0.56 μM | 1.7 μM | 5 μM | 15 μM | ( μM) |
| 998276 | 76 | 86 | 95 | 98 | NC |
| 998256 | 60 | 69 | 84 | 94 | NC |
| 998252 | 46 | 60 | 75 | 86 | 0.7 |
| 998216 | 41 | 53 | 76 | 82 | 1.1 |
| 998164 | 43 | 55 | 72 | 84 | 1.0 |
| 998048 | 35 | 46 | 67 | 82 | 1.7 |
| 998140 | 36 | 46 | 72 | 76 | 1.7 |
| 998168 | 43 | 61 | 70 | 73 | 0.8 |
| 998288 | 37 | 53 | 66 | 83 | 1.4 |
| 998253 | 48 | 65 | 81 | 93 | 0.6 |
| 998277 | 55 | 67 | 78 | 91 | NC |
| 998177 | 35 | 52 | 70 | 85 | 1.4 |
| 998157 | 41 | 60 | 75 | 87 | 0.9 |
| 998149 | 36 | 46 | 65 | 77 | 1.8 |
| 998261 | 30 | 46 | 61 | 78 | 2.2 |
| 998241 | 15 | 37 | 59 | 77 | 3.4 |
| 998069 | 28 | 42 | 64 | 79 | 2.4 |

TABLE 6
Multi-dose assay of modified oligonucleotides in 4T1 cells

| | % Inhibition RTS37638 | | | | IC50 |
|---|---|---|---|---|---|
| ION No. | 0.56 μM | 1.7 μM | 5 μM | 15 μM | ( μM) |
| 998258 | 80 | 89 | 95 | 99 | NC |
| 998150 | 69 | 86 | 91 | 95 | NC |
| 998210 | 46 | 63 | 76 | 88 | 0.7 |
| 998278 | 44 | 57 | 73 | 87 | 0.9 |
| 998082 | 55 | 64 | 74 | 86 | NC |
| 998250 | 31 | 48 | 62 | 82 | 2.0 |
| 998294 | 41 | 59 | 72 | 87 | 1.0 |
| 998254 | 25 | 42 | 63 | 81 | 2.5 |
| 998275 | 68 | 82 | 92 | 97 | NC |
| 998215 | 50 | 63 | 81 | 91 | NC |
| 998263 | 46 | 61 | 80 | 90 | 0.7 |
| 998179 | 61 | 73 | 86 | 93 | NC |
| 998279 | 44 | 57 | 75 | 89 | 0.9 |
| 998235 | 23 | 36 | 55 | 74 | 3.5 |
| 998255 | 9 | 37 | 64 | 82 | 3.1 |
| 998291 | 27 | 47 | 72 | 87 | 1.9 |

Immunoblot analysis—Adipose tissue samples were homogenized in RIPA lysis buffer containing freshly added protease (complete EDTA-free, Roche) and phosphatase (Sigma) inhibitors. Resolved proteins were transferred to PVDF membranes and subsequently probed with the following antibodies: PSMD9 (Sigma), Ser 563 pHSL (Invitrogen) and the following other antibodies from Cell Signaling Technologies: β-actin, Thr172 pAMPK, total AMPKbeta, total AMPKalpha, total ACC, Ser79 pACC, total HSL. Densitometric analysis was performed using GE Software or BioRad Quantity One software.

qPCR—RNA was isolated from adipose tissues using RNAzol reagent and isopropanol precipitation. cDNA was generated from RNA using MMLV reverse transcriptase (Invitrogen) according to the manufacturer's instructions. qPCR was performed on 10 ng of cDNA using the iTaq Universal SYBR green supermix on a QuantStudio 7 Flex (Thermofisher), using primers published previously (Bond et al., AJP Endo, 2019). Quantification of each gene was expressed by the relative mRNA level compared with control, which was calculated after normalisation to the housekeeping gene Cyclophilin a (Ppia) using the delta-CT method. Primers were designed to span exon-exon junctions and were tested for specificity using BLAST (Basic Local Alignment Search Tool; National Centre for Biotechnology Information). Amplification of a single amplicon was estimated from melt curve analysis, ensuring only a single peak and an expected temperature dissociation profile were observed.

Lipidomics—Adipose tissue was cryo-milled, suspended in PBS, sonicated and approximately 50 μg of protein in 100 of solution (5 μg/μ1) was transferred to a fresh tube. 100 of plasma was used for lipid extraction. Lipids were extracted by mixing the 10 μL sample (homogenate or plasma) with 1000 μL of butanol:methanol (1:1) with 10 mM ammonium formate which contained a mixture of internal standards. Samples were vortexed thoroughly and set in a sonicator bath for 1 hour maintained at room temperature. Samples were then centrifuged (14,000×g, 10 min, 20° C.) before transferring the into sample vials with glass inserts for analysis. Extracted lipids were processed by multiple reaction monitoring (MRM) liquid chromatography (Agilent 1290 series HPLC system and a ZORBAX eclipse plus C18 column) and tandem mass spectrometry (LC—MS/MS) on an Agilent 6490 QQQ Mass Spectrometer as previously described (Parker et al., Nature, 2019).

### EXAMPLE 1

ASO against PSMD9 caused robust silencing of PSMD9 at the protein level in white adipose tissue and activated AMPK consistent with increased energy expenditure

8-week old, male C57BL/6J and DBA/2J mice were treated twice weekly with control ASO or Psmd9 ASO at 25 mg/kg by intraperitoneal injection for 28 days. At the same time, mice were fed a Western diet (Research Diets, D12079B) (n=8 mice per group). Adipose tissue and plasma were obtained for later analysis including lipidomics, Western blotting and qPCR. During the 28-day study, body weight was measured bi-weekly. Plasma ALT and AST were analysed using a commercial kit according to the manufacturer's instructions (TECO Diagnostics). Adipose tissue samples were homogenized in RIPA lysis buffer containing freshly added protease (complete EDTA-free, Roche) and phosphatase (Sigma) inhibitors. Resolved proteins were transferred to PVDF membranes and subsequently probed with the following antibodies: PSMD9 (Sigma), Ser 563 pHSL (Invitrogen) and the following other antibodies from Cell Signaling Technologies: β-actin, Thr172 pAMPK, total AMPKbeta, total AMPKalpha, total ACC, Ser79 pACC, total HSL. Densitometric analysis was performed using GE Software or BioRad Quantity One software.

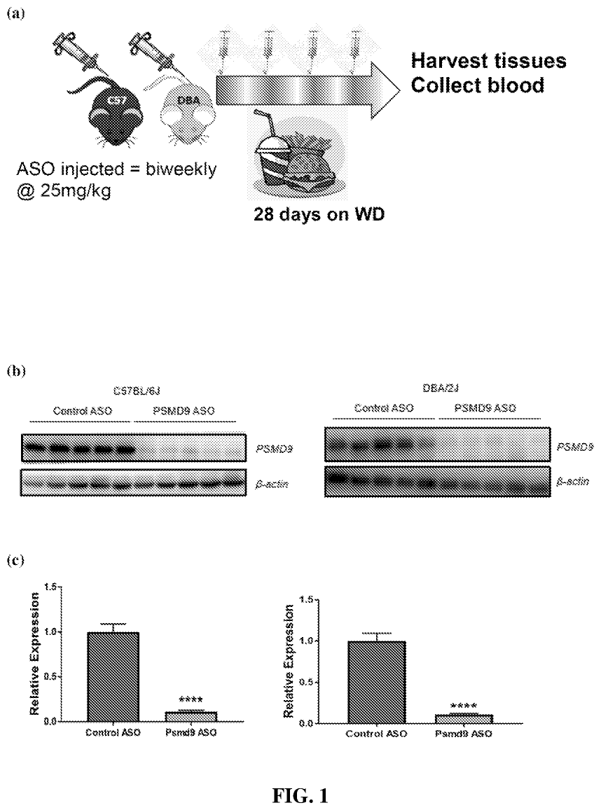

As shown in FIG. 1, ASOs against PSMD9 delivered twice weekly at 25 mg/kg for 28 days, leads to robust silencing of PSMD9 at the protein level in white adipose tissue in both strains of mice studied.

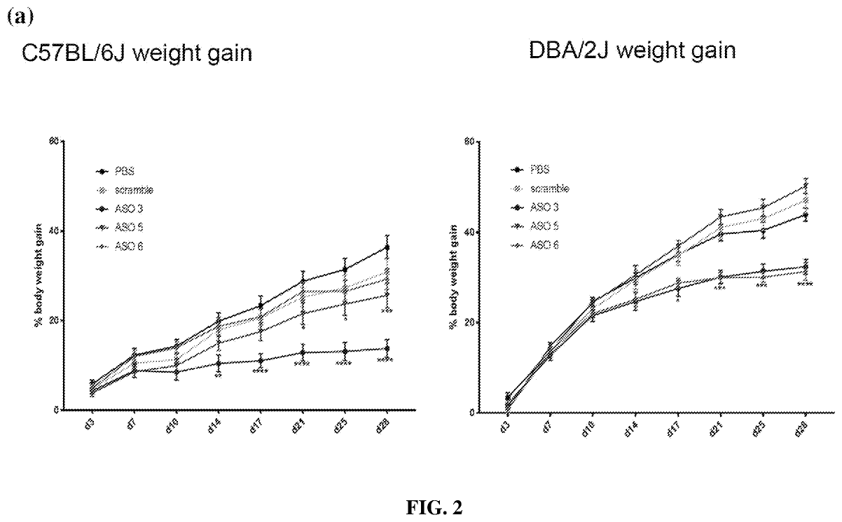

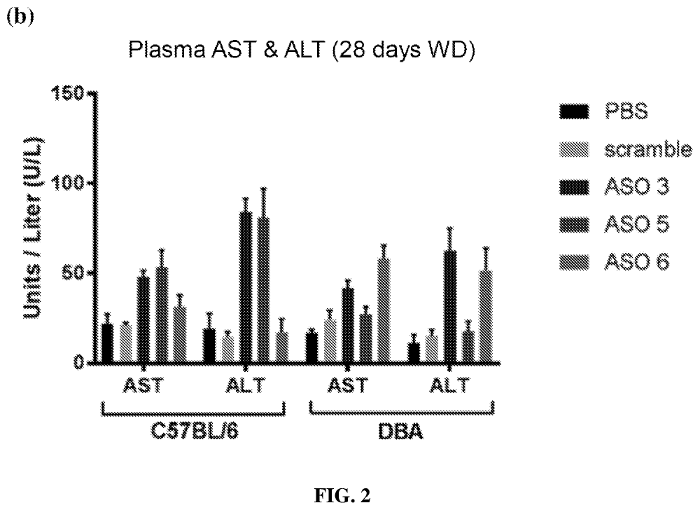

As seen in FIG. 2, ASO 3 and 5 against PSMD9 were effective at reducing weight gain in C57BL/6J mice and ASO 3 and ASO 6 against PSMD9 were effective at reducing weight gain in DBA/2J mice. None of these ASOs were associated with significant toxicity (levels >100) as assessed by plasma AST or ALT levels.

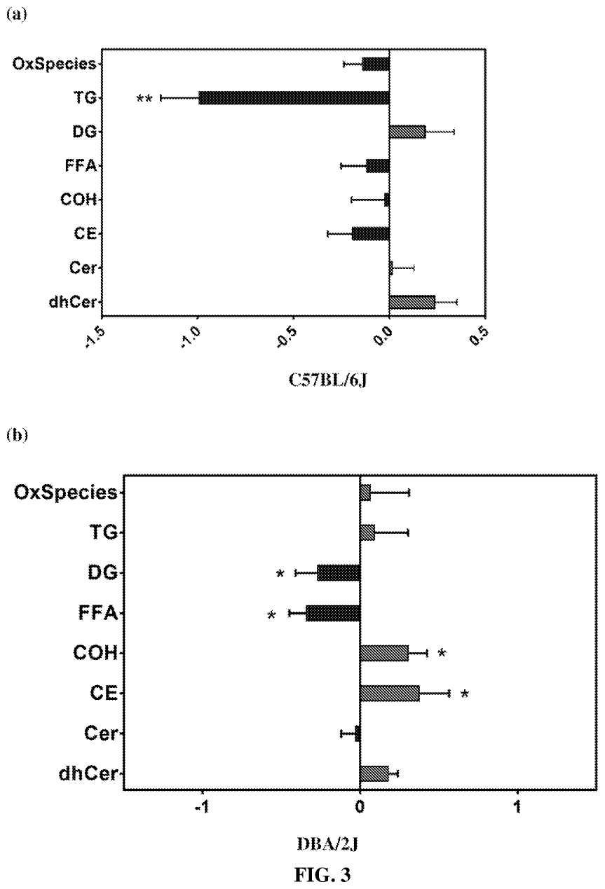

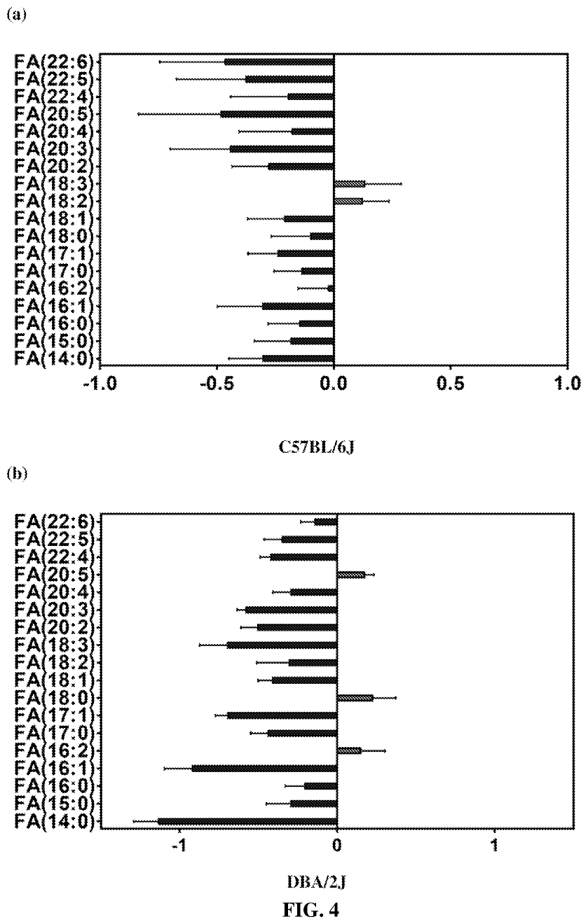

As illustrated in FIGS. 3 and 4 lipidomics analysis showed PSMD9 inhibition caused significant reductions in stored lipids in adipose tissue (e.g., TG in C57BL/6J mice) in mice after four weeks on a Western diet, as well as reductions in the abundance of fatty acids (FAs) in adipose tissue, all of which are consistent with their utilisation as a fuel source.

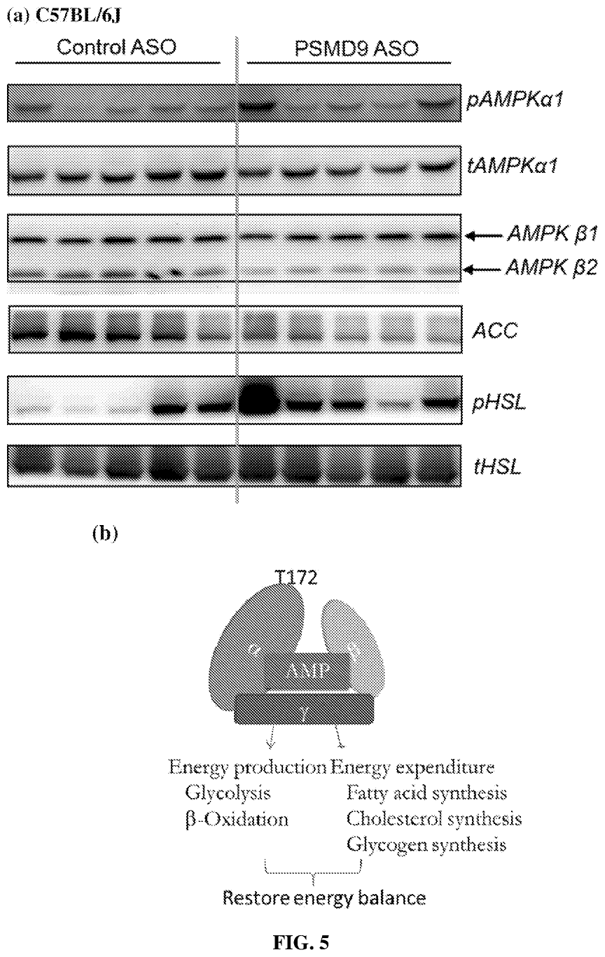

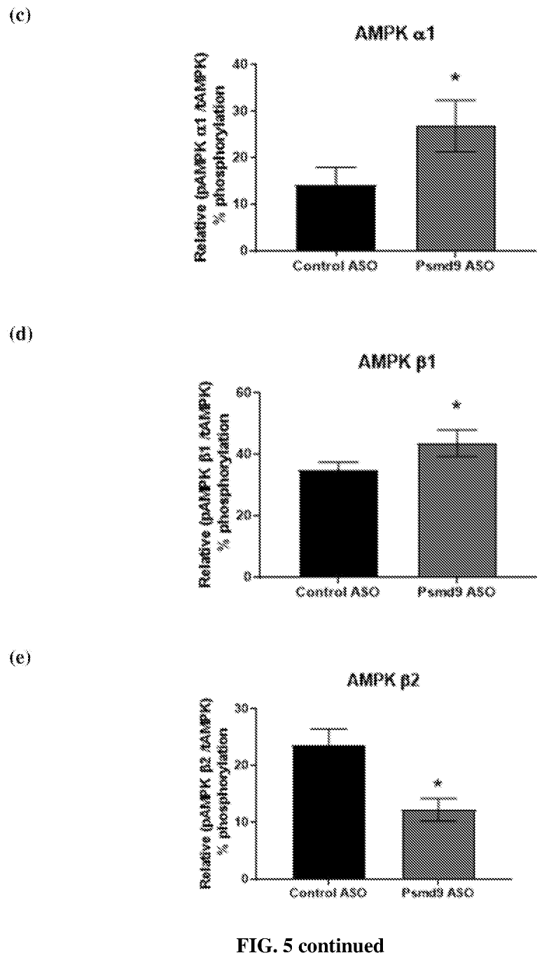

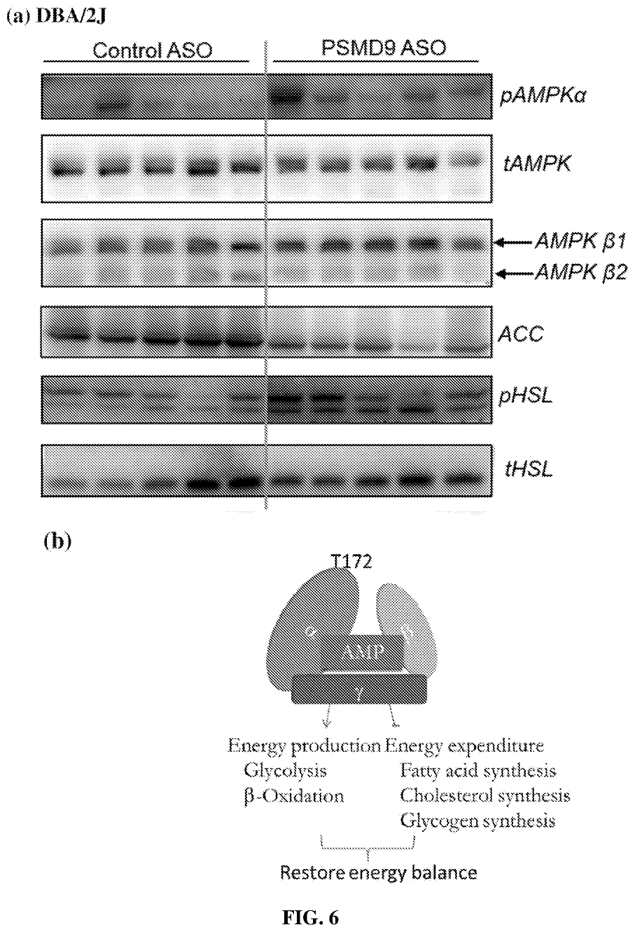

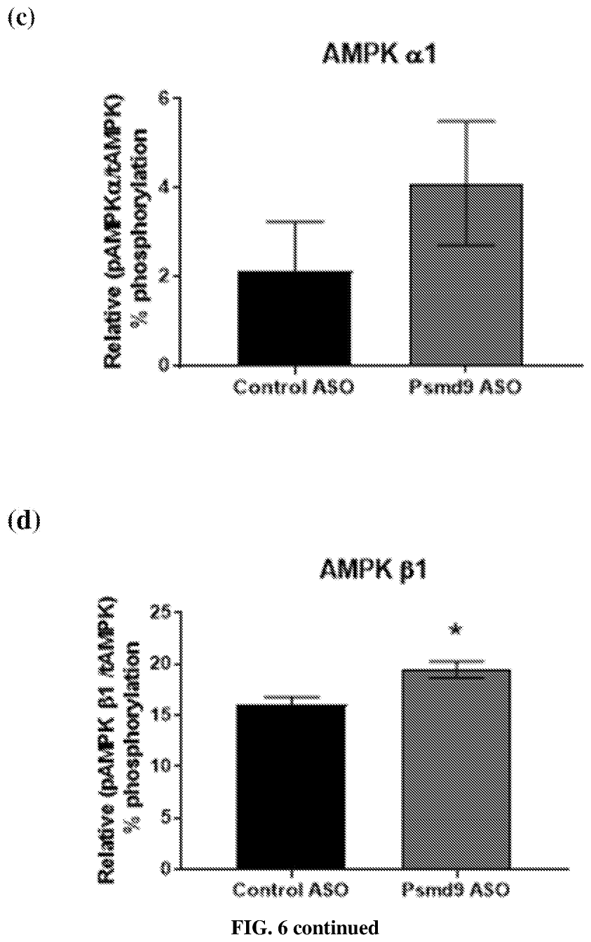

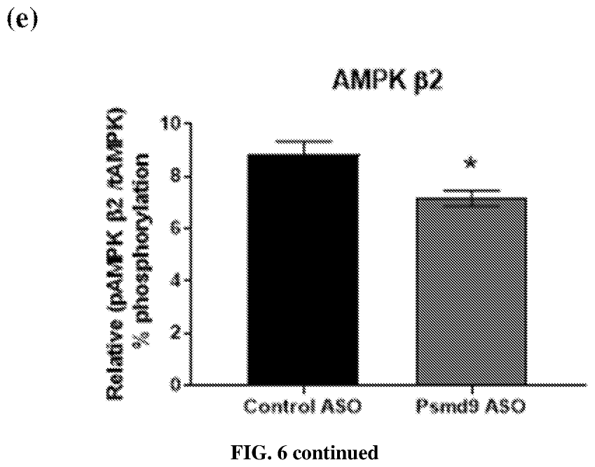

Silencing of PSMD9 was associated with robust changes in energy metabolism pathways in adipose tissue, including changes in critical enzymes such as AMP-kinase and acetyl co-A carboxylase (ACC). Specifically, increased phosphorylation of AMPKalpha at T172 (FIG. 5 (a) to (d) and FIG. 6 (a) to (d)) and decreased protein levels of ACC are consistent with a reduction in lipogenesis (synthesis of fat), an increase in lipolysis (breakdown of stored fat) and an increase in fatty acid oxidation (burning of fats for energy).

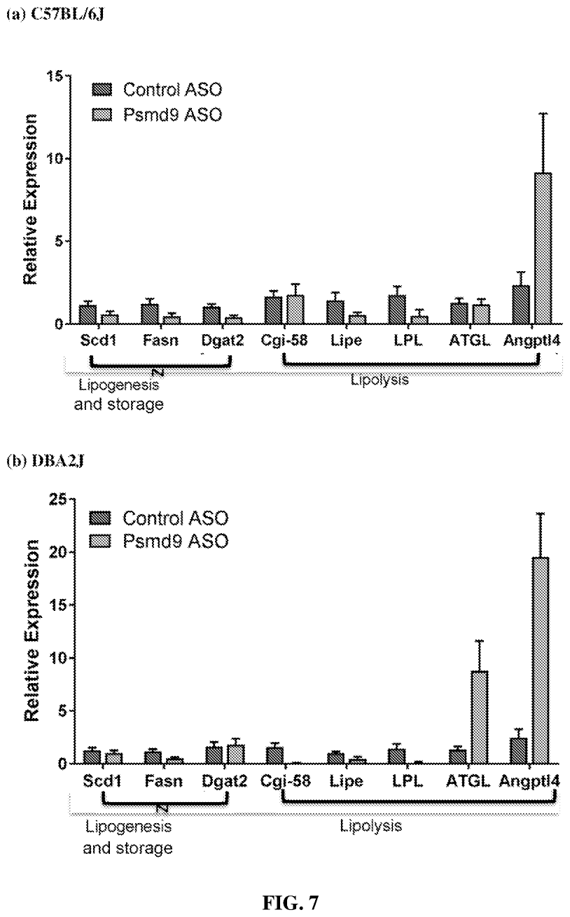

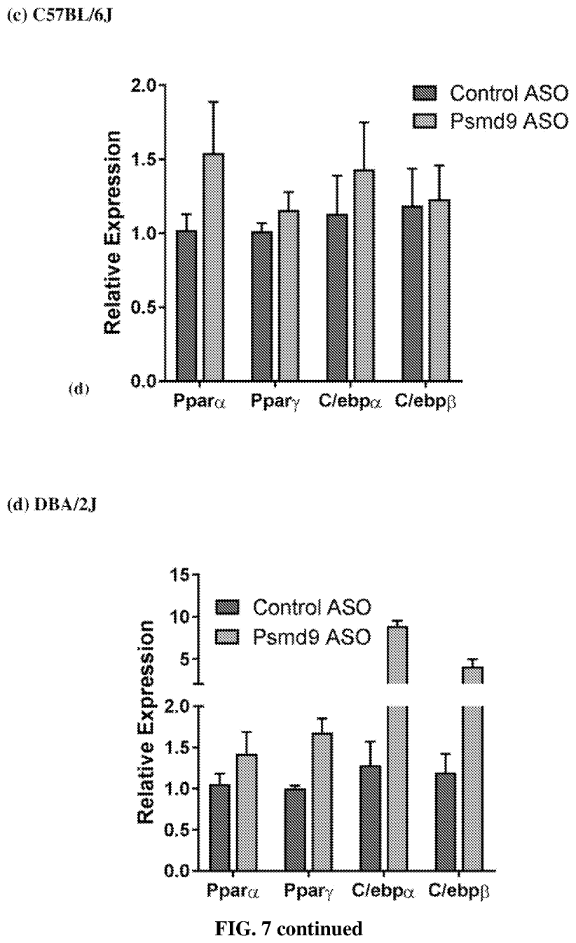

These findings were supported by qPCR analysis, which demonstrated significant alterations in the expression of genes involved in lipid metabolism and energy utilisation, particularly those involved in lipogenesis, lipolysis and oxidation. mRNA expression levels in adipose tissue in control and ASO treated mice were assessed after four weeks of ASO administration and feeding of a western diet. As illustrated in FIG. 7 (a) to (d) mice showed alterations in expression of genes involved in lipogenesis, lipolysis and fatty acid oxidation.

In conclusion, reduction or silencing of PSMD9 in adipose tissues of mice on a Western diet leads to a reduction in stored lipid, through a mechanism including increased lipolysis and fatty acid oxidation. These surprising findings show that sustained silencing of PSMD9 will be a useful method for reducing adipose tissue mass and thus adiposity in mammals.

### EXAMPLE 2

Further experiments to assess and confirm the effects of PSMD9 silencing in adipose tissue include comparing Native vs Gal-Nac targeted ASOs. This study will compare native ASOs that are untargeted to a cell type to ASOs with a “GalNAc” conjugate, which targets the ASO to the liver for uptake by the asialoglycoprotein receptor (ASGR). These studies will allow us to determine whether the effects observed with the ASO are a result of direct effects on the liver or whether the effects are also a result of targeting extra-hepatic tissues, such as adipose tissue. Mice (C57BL/6J and in DBA/2J) experiments will run for 6 months and mice will be concurrently fed the AMLN diet (40% total fat kCal: 18.5% trans-fat, 20% fructose, 2% cholesterol) considered to be a gold-standard diet-induced model of non-alcoholic steatohepatitis (NASH). The mice will also gain weight and develop complications including type 2 diabetes. Mice will undergo the protocol illustrated below, with assessment of body weight, fat mass (EchoMRI) and glucose tolerance (GTT) as well as bleeds to assess fasting glucose, insulin and lipids. Mice will also be placed in metabolic cages (Promethion) to assess food intake and energy expenditure. Readouts of hepatic fibrosis, inflammation and ER stress will also be assessed. Subsequent studies using a regression model in which administration of ASOs will commence after a 6-month period of feeding the AMLN diet will be performed. Mice will be concurrently fed AMLN diet whilst receiving ASOs. GTT=glucose tolerance test. N=10/group.

Additionally, tissue-specific deletion of PSMD9 using PSMD9 floxed mouse-preventative and treatment outcomes is conducted. Using CRISPR/Cas9 technology, a PSMD9 floxed mouse on a C57BL/6J background has been generated. These mice are bred with iAdipoQ-Cre mice to establish a model to determine the effect of adipose-specific deletion of PSMD9. Concurrent studies are carried out on mice with liver-specific deletion of PSMD9 (PMSD9 floxed x Albumin-Cre). Together, these studies will allow us to determine the tissue-specific effects of genetic deletion of PSMD9 on adipose tissue function.

### EXAMPLE 3 FURTHER STUDIES

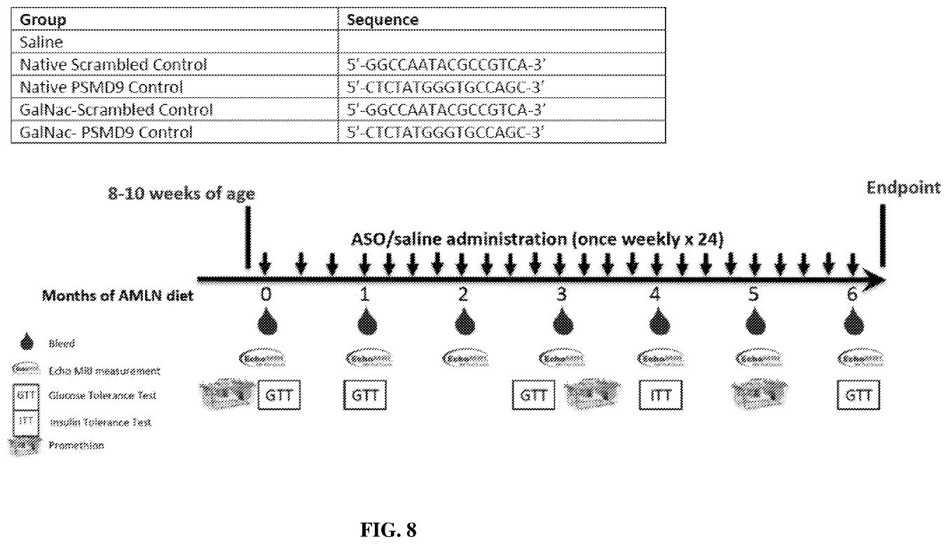

At 8-10 weeks of age, male C57BL/6J mice were fed an AMLN diet (43% kCal fat; 20% fructose, 2% cholesterol) for a duration of 6 months (See study design in FIG. 8). Mice were administered one of the anti-sense oligonucleotides (ASOs) as listed in the table in FIG. 8 or saline, weekly via intraperitoneal injection. Native ASO was administered at 25 mg/kg and liver targeted ASO was administered at 1 mg/kg once weekly. Both the Native (whole body) and GalNac (liver targeted) ASOs bind the same sequence of the PSMD9 mRNA (ASO 3). ASO were designed and synthesized by Ionis Pharmaceuticals. Chimeric 16-oligonucleotide phosphorothioate oligonucleotides targeted to mouse PSMD9 (5′-CTCTATGGGTGCCAGC-3′ SEQ ID NO:6) or control; N-acetylgalactosamine(GalNac) conjugation targets delivery to hepatocytes via the asialoglycoprotein receptor (ASGR). Over the duration of the study mice underwent extensive metabolic phenotyping. The data illustrated present data on body composition (echoMRI) and glucose tolerance (2 mg/kg lean mass) at ˜1 week prior to study end. All other data is from study end point.

Body composition was assessed by EchoMRI (Echo Medical Systems). Glucose tolerance was assessed by an oral glucose tolerance test. Glucose (2 mg/kg lean mass) was administered by gavage and blood glucose assessed over time as indicated. Plasma biomarkers were assessed by ASAP Laboratories.

RNA isolation and quantitative RT-PCR (qPCR): Tissue were homogenised in RNAzol, then RNA isolated using choloroform followed by isopropanol precipitation. Pellets were washed with 75% ethanol and resuspended in molecular grade water. Complimentary DNA (cDNA) was generated using M-MLV reverse transcriptase (Invitrogen). qPCR was performed on 10 ng of cDNA with iTap Universal SYBR Green Supermix (Bio-Rad) using a QuantStudio 7 Flex Thermocycler (ThermoFisher Scientific). Data was analysed using the ΔΔCt method, normalized to PPIA (cyclophilin B) and expressed as fold over saline.

Protein Isolation and Western Blotting: Protein lysate was homogenised in radioimmunoprecipitation (RIPA) buffer supplemented with protease inhibitors and separated on an SDS-PAGE gel then transferred to a PVDF membrane. Membranes were blocked with 3% milk for 2 hours then incubated overnight at 40C with primary antibody as indicated. After washing, membranes were incubated with HRP-conjugated secondary antibodies (Bio-Rad) for 2 hours and then visualised using chemiluminescence (Pierce). Image Lab (Bio-Rad, version 5.2.1 build 11) was used to perform densitometry analysis and expression normalised to the corresponding protein loading control for each individual sample.

Statistics: Students t-test or Mann-Whitney U test—*p<0.05, **p<0.01, ***p<0.001, ****p<0.0001 vs Native/GalNac Control as indicated. Percentage change is from Native/GalNac Control as indicated

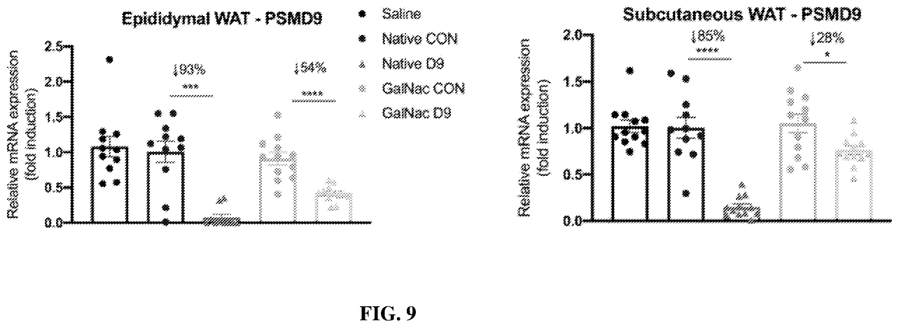

Firstly, to confirm the efficacy of the ASOs, PSMD9 mRNA expression in adipose tissue was assessed at the end of the study (FIG. 9). A robust attenuation of PSMD9 expression was observed in both epididymal (visceral/central) and subcutaneous adipose tissue with the Native PSMD9 ASO, with no expression detected in some samples at the mRNA level. Interestingly, a partial reduction in PSMD9 mRNA expression was observed with the GalNac PSMD9 ASO in both epididymal (central) and subcutaneous (peripheral) WAT, consistent with this ASO harbouring a liver homing moiety.

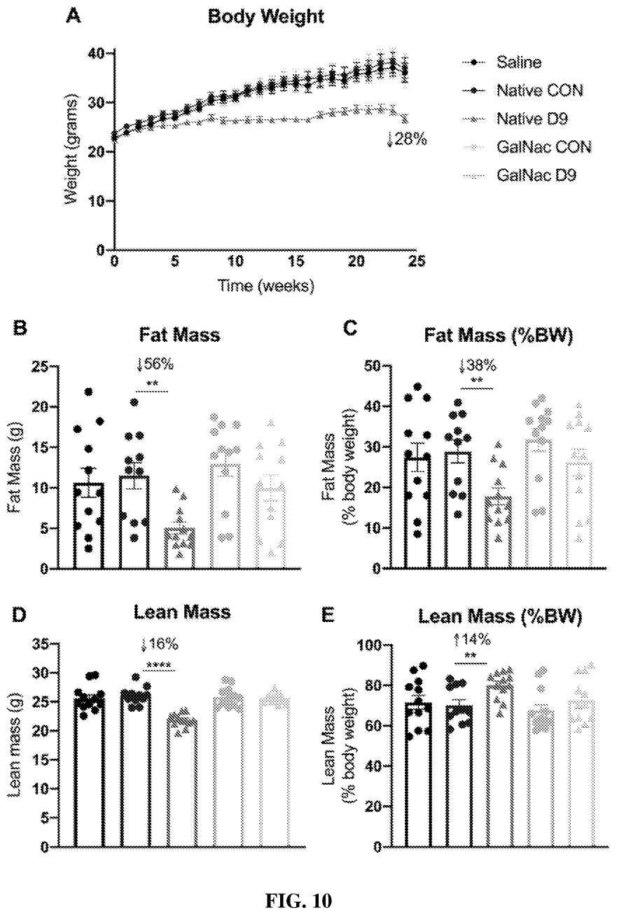

Body weight and body composition. As can be seen in FIG. 10, there is a significant reduction in the propensity to gain weight only in mice administered the native ASO against PSMD9 (FIG. 10A). In comparison, in mice that were administered the same ASO but targeted to the liver (GalNac D9), no prevention in weight gain relative to its respective control or the saline treated group was observed. Upon analysis of body composition by EchoMRI, a marked reduction in fat mass was observed in mice administered the Native PSMD9 ASO, but not those administered the liver targeted PSMD9 ASO (FIG. 10B,C). These mice also exhibited a small reduction in lean mass (FIG. 10D), however relative to body weight, this was reflected as an increased lean mass due to the overall reduction in body weight primarily driven by a change in fat mass in this group (FIG. 10E). In one embodiment, the effects of PSMD9 reduction on adiposity are independent of effects seen in the liver. The results described herein are also not predicable from the results of studying the effects of PSMD9 inhibitors on liver, or the effects of inhibiting key enzymes involved in de novo lipogenesis (synthesis of new lipids—DNL).

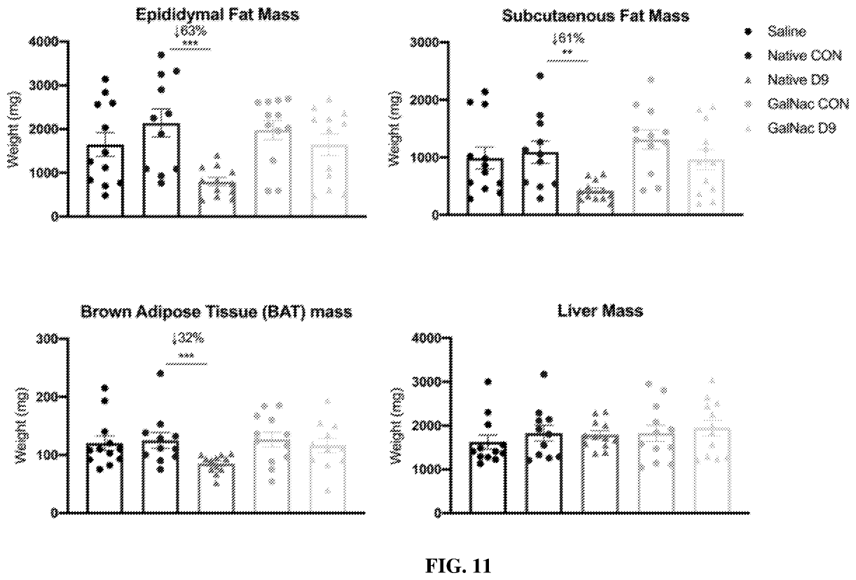

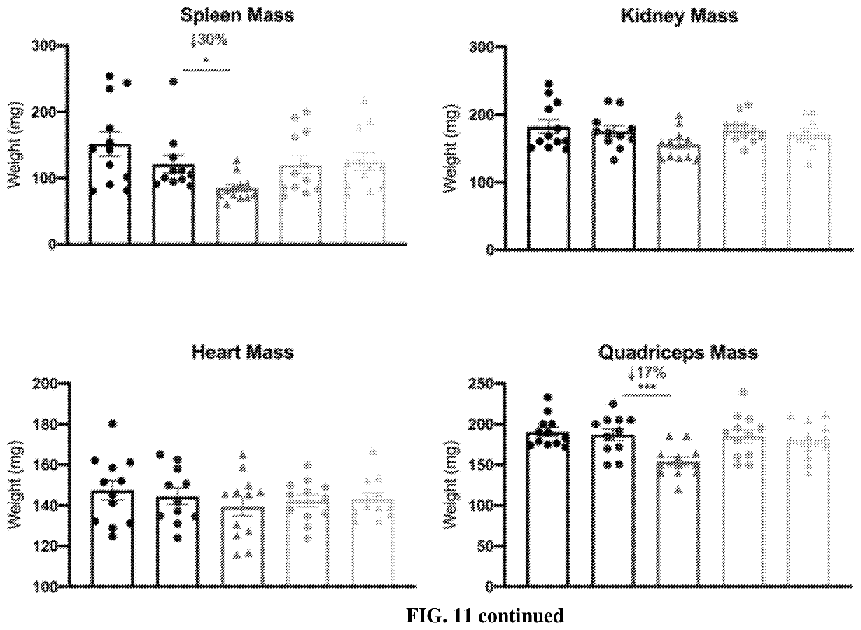

Assessment of organ weights, as seen in FIG. 11, demonstrated a reduction in epididymal and subcutaneous adipose mass with the native PSMD9 ASO, consistent with the assessment of body fat composition in FIG. 10. A small reduction in brown adipose tissue mass was also noted, suggesting that native ASOs also target this adipose tissue depot. Native PSMD9 ASO was also associated with a reduction in spleen and skeletal muscle mass (isolated quadriceps), consistent with the reduction in lean mass observed in FIG. 10. In contrast, there were no changes in heart or kidney weight (FIG. 11), indicating that the ASO targeting of PSMD9 do not affect these organs.

### EXAMPLE 4

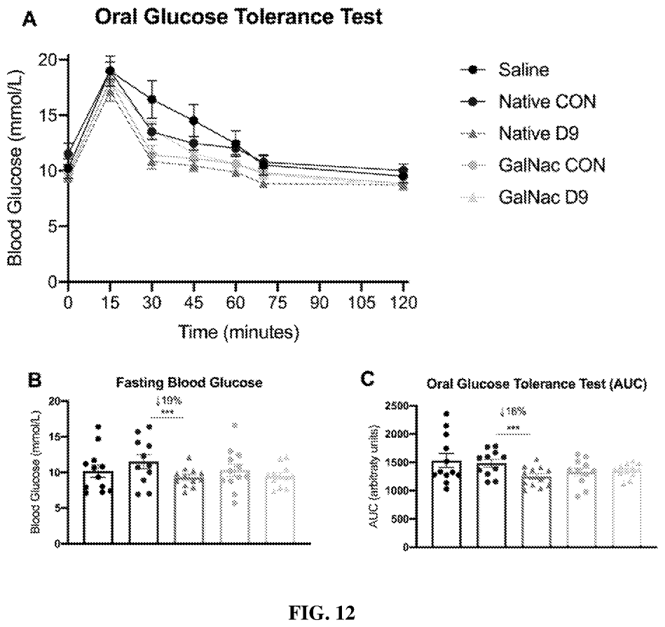

Following the observation that adipose tissue weights were altered, an assessment was made of whether there were any alterations in glucose metabolism in these mice. Fasting blood glucose readings and oral glucose tolerance tests were performed. These studies revealed a small but significant improvement in fasting blood glucose levels (FIG. 12A,B) in mice receiving the Native PSMD9 ASO compared to their control counterparts. Moreover, when mice underwent a glucose tolerance test, those mice that received the Native PSMD9 ASO returned to basal glucose levels faster than those that receive the Native Control ASO (FIG. 12A,C; light blue group), indicative of improved glucose handling.

### EXAMPLE 5

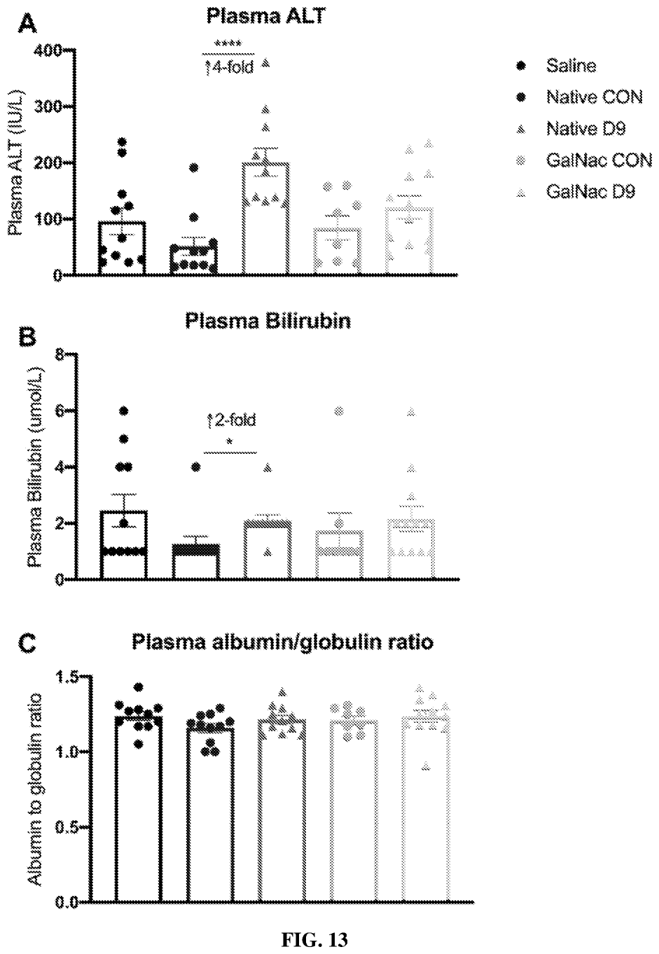

To investigate potential toxicity effects of the ASO treatments, a number of measures in the blood of each treatment group were taken. Plasma levels of ALT, bilirubin and albumin to globulin ratio, which are clinically relevant biomarkers of liver and kidney toxicity respectively were assessed. Although a slight increase in plasma ALT was observed with Native PSMD9 ASO (FIG. 13A), there was no evidence of toxicity with regard to bilirubin levels or albumin to globulin ratio (FIG. 13B,C).

### EXAMPLE 6

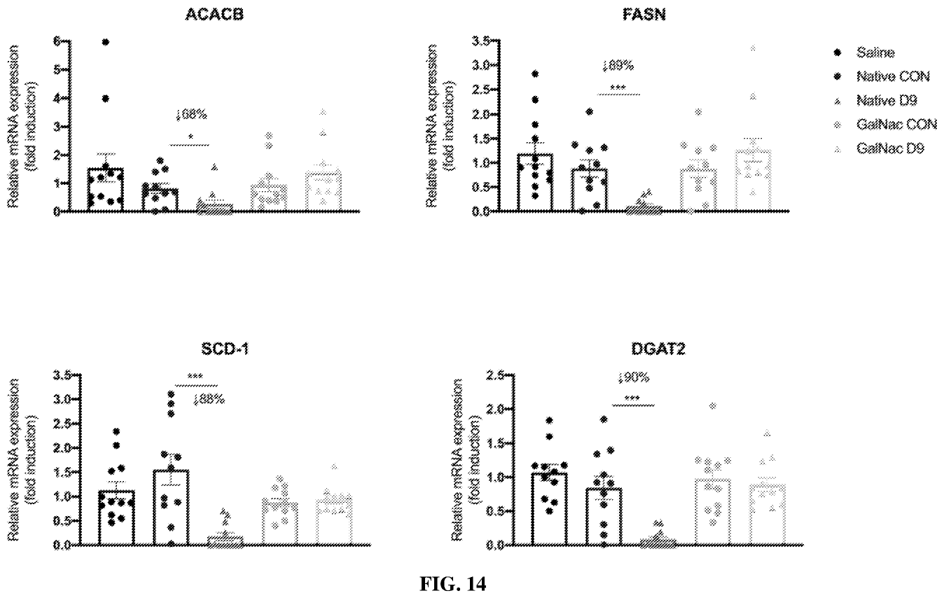

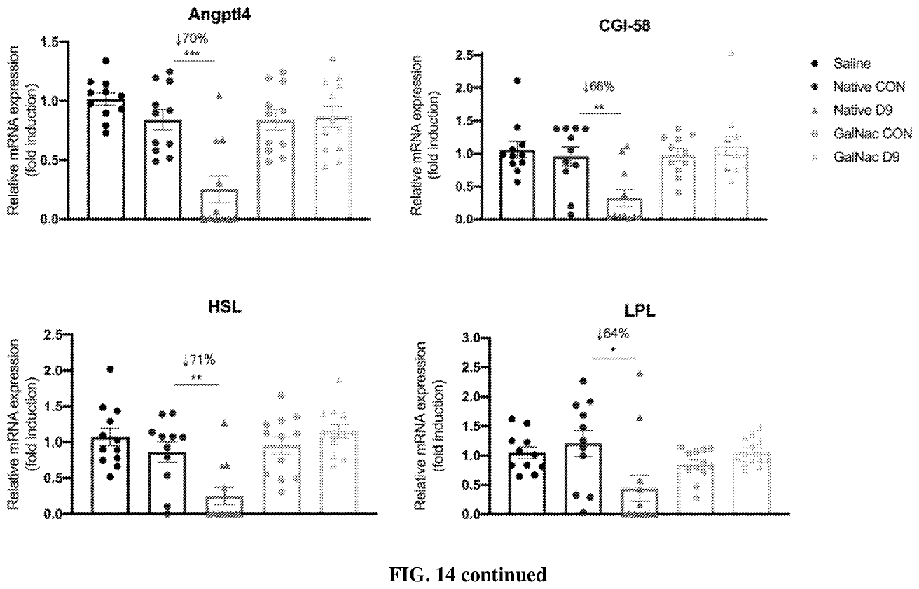

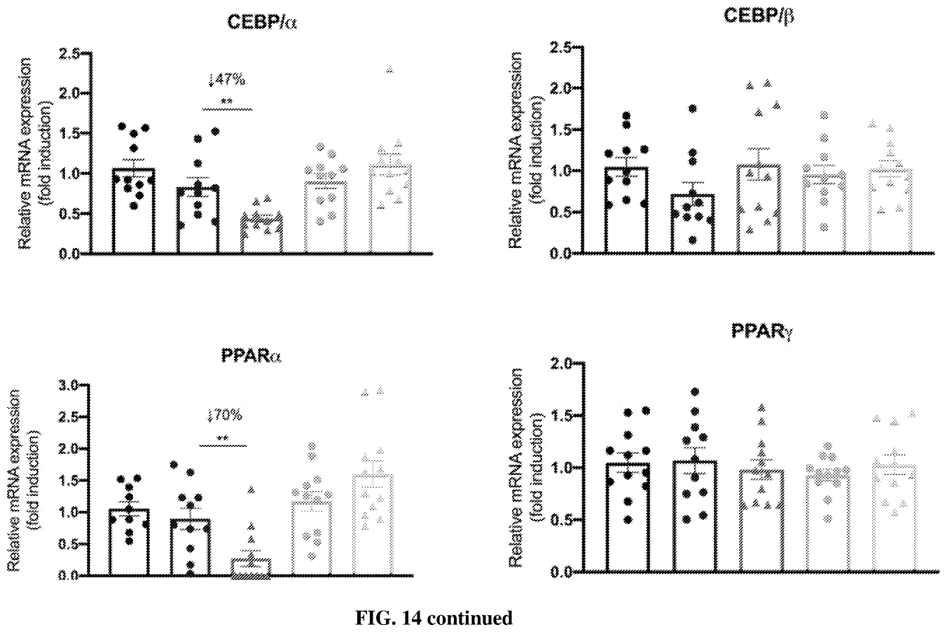

In light of the marked effect of Native PSMD9 ASO on adipose tissues mass, the molecular changes occurring in both epididymal and subcutaneous adipose depots were investigated. In epididymal white adipose tissue (WAT), administration of Native PSMD9 ASO led to significant reductions in the expression of genes associated with lipid synthesis (ACACB, FASN, SCD1) and storage (DGAT2; FIG. 14). Furthermore, reductions in Angptl4-LPL axis were observed which is involved in the hydrolysis of circulating lipids, reductions in the hydrolysis of lipid stores (CGI-58, HSL), a reduction in CEBP/α, which has been shown drive the formation of new fat cells, as well as and a reduction in PPARα, which drives the utilisation of lipids for energy. Essentially, all lipid regulatory pathways that were assessed in adipose tissue were reduced by the native PSMD9 ASO, consistent with reduced lipid burden.

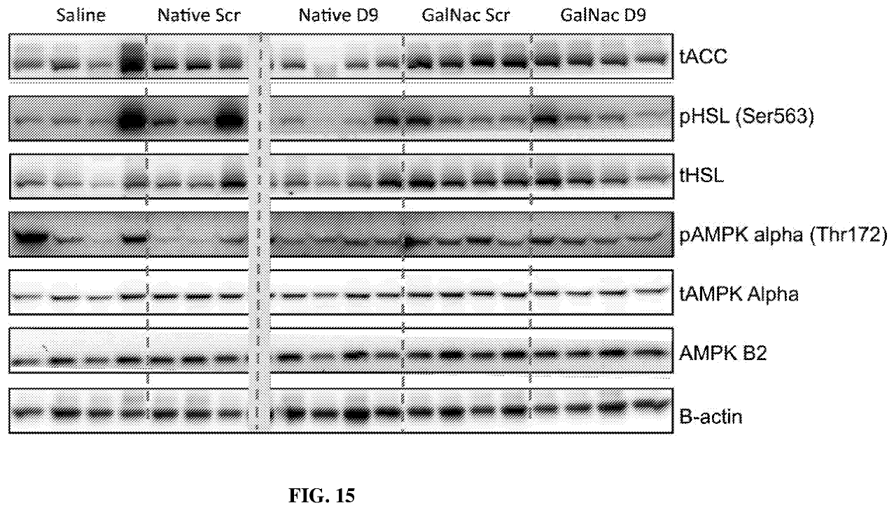

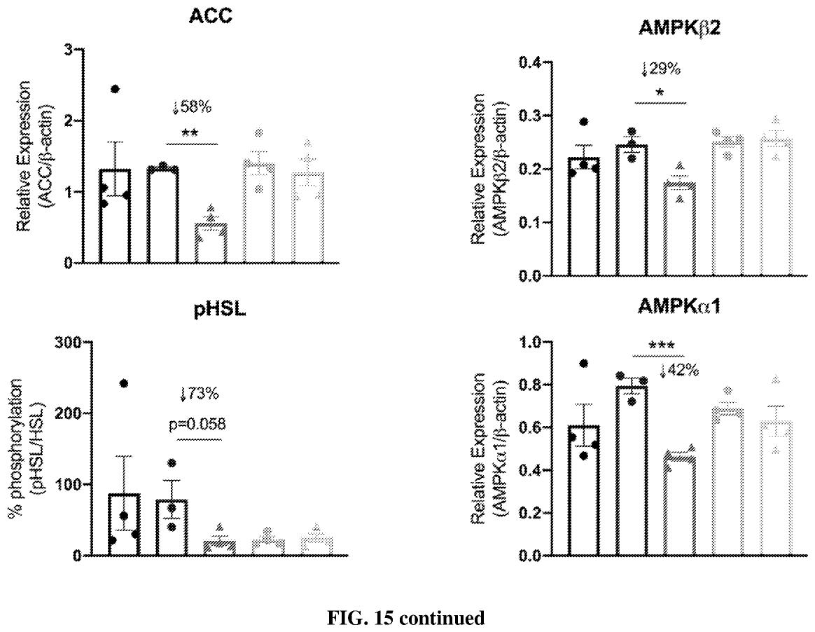

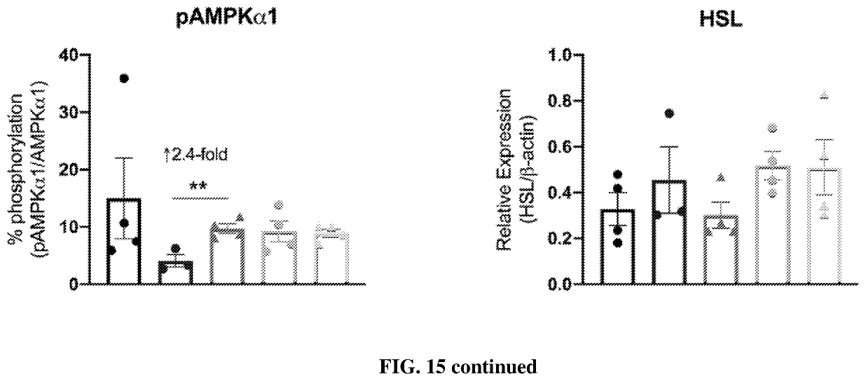

Further to changes in gene expression, changes to protein levels and activity in the adipose tissue were assessed. Consistent with alterations in mRNA levels (Acacb), reductions in ACC were confirmed at the protein level (FIG. 15). Reductions in protein expression of AMPKα, AMPKβ2 and an increase in AMPKα phosphorylation (pAMPK) were also observed, which indicates an upregulation of catabolism. A trend for a reduction in phosphorylation of HSL (pHSL), demonstrating altered lipolysis activity was identified.

Subcutaneous white adipose tissue was also assessed. It was important to determine the effects of the ASO in two different fat depots to understand if the effects were consistent. Moreover, the epididymal fat pad is exposed to a high level of ASO given its location in the peritoneum, where the drug was injected. The subcutaneous adipose depot would only receive drug from exposure via the circulation, which is better measure of the difference in targeting of the Native vs the GalNAc ASOs.

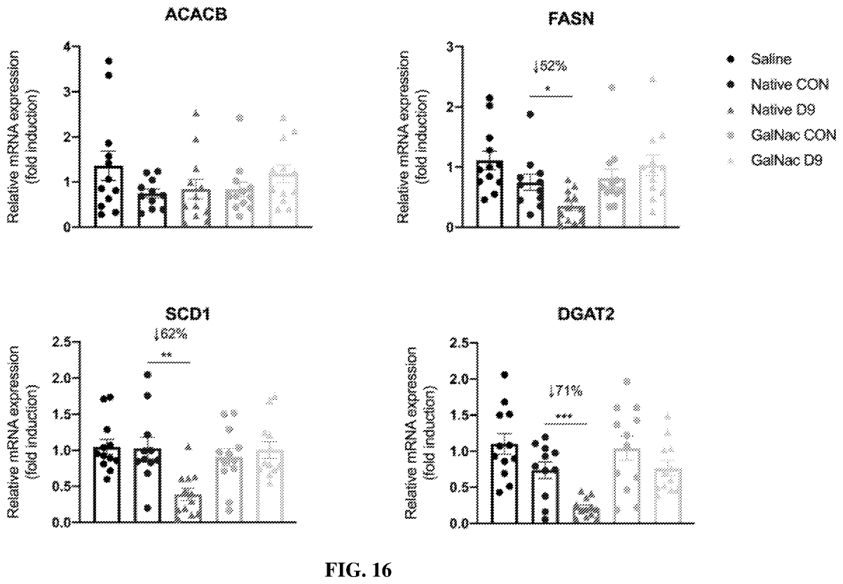

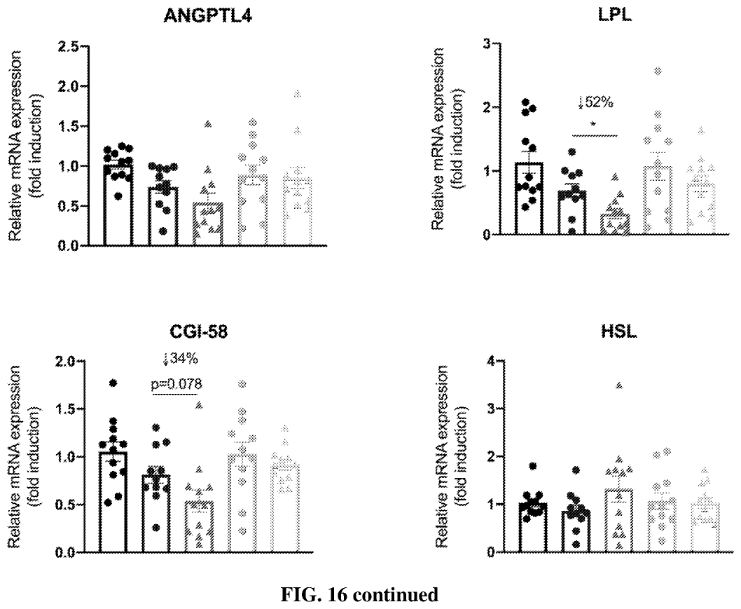

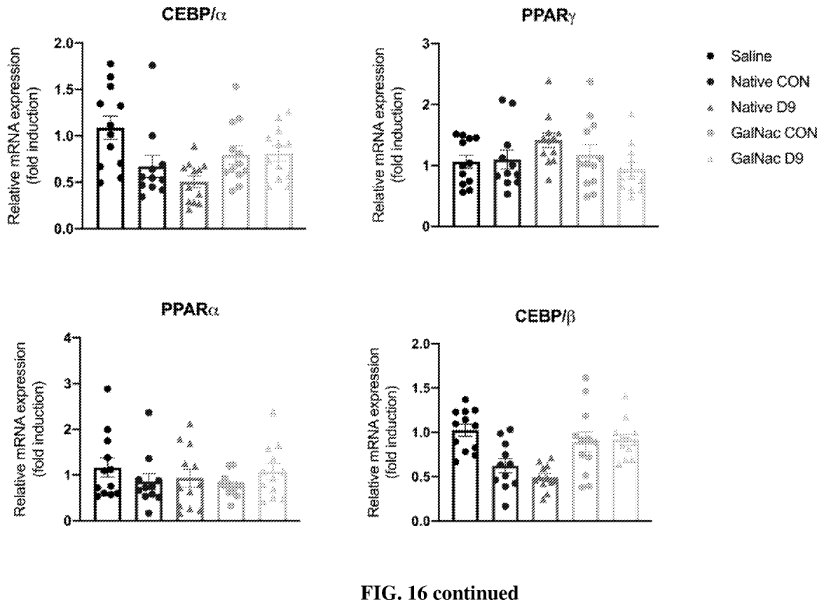

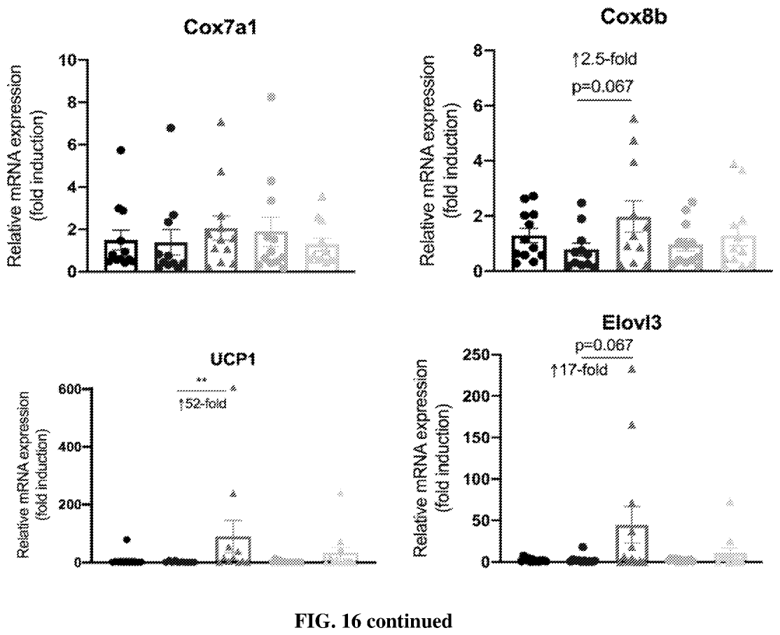

Similar effects to those seen in epididymal WAT (FIG. 14) were observed in subcutaneous WAT, although not always to the same degree. Indeed, administration of Native PSMD9 ASO led to significant reductions in the expression of genes associated with lipid synthesis (FASN, SCD1) and storage (DGAT2; FIG. 16). Furthermore, reductions were observed in LPL and a trend for a reduction in CGI-58, involved in the hydrolysis of circulating lipids and lipid stores respectively.

Of particular interest was the effect of Native PSMD9 ASO on genes associated with adipocyte browning (FIG. 16) a phenomenon associated with improved metabolic activity. Indeed, although variable, increases in Cox8b, UCP1 and Elov13, all classical genes associated with browning were observed. These data suggest that silencing of PSMD9 in WAT drives the conversion to brown adipose tissue, which would result in favourable changes in metabolism and energy expenditure. The data provided herein provides evidence that silencing of PSMD9 in adipose tissue via ASO is associated with beneficial changes to whole body metabolism that lead to improvements in obesity and its complications.

Specifically, silencing of PSMD9 in adipose tissue via a native ASO led to reduced weight gain on a diet high in fat, as well as reduced fasting blood glucose levels and improved glucose tolerance. Furthermore, molecular changes in adipose tissue consistent with reduced lipid synthesis, the promotion of catabolic processes to generate energy and enhanced energy expenditure were observed. For example, there was a 2 to 50 fold increase in the expression of enzymes associated with WAT browning and increased metabolic activity.

The observed molecular changes support and enable the practise of the herein described methods and uses to prevent or reduce adiposity in a subject in need thereof.

The observed pathway activation, for example, would promote weight loss as follows:

Increased phosphorylation of AMPK in adipose tissue alters lipolysis and the burning of fat for energy, leading to reductions in fat mass (Daval et al, J Physiol, 2006)

Increased UCP1 expression and “browning” in white adipose tissue is associated with increased energy expenditure and weight loss in humans (Bettini et al, Frontiers in Endocrinology 2019; 10:548, Finlin et al, JCI, 2020 PMID: 31961829)

Reduced de novo lipogenesis (fat synthesis) in adipose tissue is associated with weight loss and improved glucose control (Hyun et al, BBRC, 2010; Abu-Elheiga et al, JBC, 2012; Harriman et al, PNAS, 2016).

Thus, the data described herein support and enable a role for reduction or silencing of PSMD9 in adipose tissue to:
Prevent weight gain and the accumulation of fat tissue in the setting of excess caloric intake
Prevent weight gain and the accumulation of fat tissue in the setting of other forms of obesity such as that caused by sedentary behaviour or genetic predisposition.
Reduce excess weight and fat mass in the setting of pre-existing obesity induced by excess caloric intake, sedentary behaviour or genetic predisposition
Improve blood glucose levels and reduce adiposity in individuals who are obese and have glucose intolerance, insulin resistance or type 2 diabetes
Convert white adipose tissue from a storage unit for fat, to a tissue that burns fat for energy
Activate cellular pathways in adipose tissue that liberate fat from intracellular stores (lipolysis) for the purposes of energy production
Reduce in the risk of other complications associated with obesity such as glucose intolerance, and insulin resistance, and fatty liver disease and cardiovascular disease.

TABLE 7

| SEQ ID NO. | Description |
|---|---|
| SEQ ID NO: 1 | human PSMD9 nucleic acid sequence GenBank NM-002813 2368 nucleotides |
| SEQ ID NO: 2 | human PSMD9 amino acid sequence encoded by SEQ ID NO: 1 |
| SEQ ID NO: 3 | mouse PSMD9 nucleic acid sequence GenBank NM-026000 |
| SEQ ID NO: 4 | mouse PSMD9 amino acid sequence |
| SEQ ID NO: 5 | polynucleotide sequence of mouse PSMD9 CDS included in adenovirus for |
| | overexpression studies |
| SEQ ID NO: 6 | nucleotide sequence of ASO 3 directed against mouse PSMD9 |
| SEQ ID NO: 7 | nucleotide sequence of ASO 5 directed against mouse PSMD9 |
| SEQ ID NO: 8 | nucleotide sequence of ASO 6 directed against mouse PSMD9 |
| SEQ ID NO: 9 | nucleotide sequence of scrambled ASO control |
| SEQ ID NO: 10 | nucleotide sequence of mouse PSMD9 mRNA GenBank NM-026000.2 |
| SEQ ID NO: 11 | nucleotide sequence of mouse PSMD9 genomic sequence GenBank no. NC- |
| | 000071.6 truncated sequence of target region nucleotides 123225001 to 123253000 |
| SEQ ID NO: 12 | forward primer for probeset RTS37638 PSMD9 |
| SEQ ID NO: 13 | reverse primer for probeset PSMD9 |
| SEQ ID NO: 14 | probe for probeset PSMD9 |

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety

Those of skill in the art will appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

Abu-Elheiga et al, JBC, 2012
Alshehry Z. H. et al. (2015) Metabolites 5, 389-403
Alshehry Z. H. et al. (2016) Circulation 134, 1637-1650
Andreux P. A et al. (2012) Cell 150, 1287-1299.
Azimifar S. B. et al. (2014) Cell metabolism 20, 1076-1087
Bennett B. J et al. (2010) Genome research 20, 281-290
Bettini et al, Frontiers in Endocrinology 2019; 10:548
Chick J. M. et al. (2016) Nature 534:500-505
Churchill, G. A et al. (2004) Nature Genetics 36, 1133-1137
Churchill G. A. et al. (2012) Mammalian genome: official journal of the International Mammalian Genome Society 23, 713-718
Daval et al, J Physiol, 2006
Cox J. and Mann M. (2008) Nature Biotechnology 26, 1367-1372
Drew, B. G. et al. (2015) The Journal of biological chemistry 290, 5566-5581
Eng J. K. et al. (1994) Journal of the American Society for Mass Spectrometry 5, 976-989
Finlin et al, JCI, 2020 PMID: 31961829
Ghazalpour et al. (2012) Mammalian genome: official journal of the International Mammalian Genome Society 23, 680-692
Garzon J. I., et al. (2016) eLife 5
Harriman et al, PNAS, 2016
Harris R., et al. (2017) The Lancet Gastroenterology & hepatology 2, 288-297
Hvam et al. Molecular Therapy 25(7) July 2017.
Jiang et al. J Clin Invest. 2005 April; 115(4):1030-8. Epub 2005 Mar. 10
Hyun et al, BBRC, 2010
Langfelder P. and Horvath, S. (2008) BMC bioinformatics 9, 559
Luck, S., et al. (2014) Cell reports 9, 741-751
Mota et al. Metabolism 65(8):1049-1061, 2016
Musso et al. Nature Reviews, Drug Discovery 15: 249-274, 2016
Parker et al, Nature 567(7747):187-193, 2019 published Feb. 27 2019
Parks B. W et al. (2013) Cell metabolism 17, 141-152
Parks B. W et al. (2015) Cell metabolism 21, 334-346
Prakash et al. J. Med. Chem 2016, 59, 2718-2733
Singh et al PloS One (2016) 0164133
Tacer and Rozman J. Lipids (2011) 783976
Watanabe T. K. et al. (1998) Genomics 50, 241-250
Williams E. G et al. (2016) Science 352, aad0189
Wu Y et al. (2014) Cell 158, 1415-1430
Xing Xian Yu et al. Hepatology; 42:362-371, 2005.

1. A method of reducing adiposity, reducing adipose weight gain or promoting adipose weight loss in a mammalian subject, comprising administering a PSMD9 inhibitor to the subject.

2. The method of claim 1, wherein the PSMD9 inhibitor is or comprises a peptide, a peptidomimetic, a small molecule, a polynucleotide, or a polypeptide.

3. The method of claim 2 wherein the peptide is a phosphopeptide or phosphomimetic.

4. The method of claim 2, wherein the polypeptide comprises an anti-PSMD9 antibody or an antigen binding fragment thereof.

5. The method of claim 2, wherein the PSMD9 inhibitor is a polynucleotide.

6. The method of claim 5, wherein the polynucleotide is a modified oligonucleotide targeting PSMD9.

7. The method of claim 6, wherein the compound is single-stranded or double stranded.

8. (canceled)

9. The method of claim 6, wherein the modified oligonucleotide comprises at least one modification selected from at least one modified internucleoside linkage, at least one modified sugar moiety, and at least one modified nucleobase.

10. The method of claim 6, wherein the modified oligonucleotide comprises:
A gap segment consisting of linked deoxynucleotides;
A 5′ wing segment consisting of linked nucleosides;
A 3′ wing segment consisting of linked nucleosides;
wherein the gap segment is positioned immediately adjacent to and between the 5′ wing segment and the 3′ wing segment and wherein each nucleoside of each wing segment comprises a modified sugar.

11. The method of claim 2, wherein the PSMD9 inhibitor is an iRNA, such as an shRNA, siRNA, miRNA.

12. The method of claim 5 wherein the polynucleotide is a vector for the expression of the PSMD9 inhibitor.

13. The method of claim 12, wherein the vector is a viral vector.

14. The method of claim 13, wherein the viral vector is an adenoviral vector.

15. The method of claim 1, wherein the PSMD9 inhibitor is administered in an amount and over a time effective to reduce adipose tissue weight gain or promoting adipose tissue weight loss in the subject.

16. The method of claim 1, wherein the PSMD9 inhibitor is administered in an amount effective increase one or more of lipolysis, fatty acid oxidation, lipid metabolism or decrease lipogenesis in adipose tissue in the subject.

17. The method of claim 15, wherein the adipose tissue is visceral adipose tissue.

18. The method of claim 1, further comprising measuring weight loss or reduced weight gain over a period of time.

19. A PSMD9 inhibitor for use, or for use in the manufacture of a medicament for use, in reducing adipose tissue in a subject in need thereof.

20. A pharmaceutical composition comprising a PSMD9 inhibitor and a pharmaceutically acceptable carrier and/or diluent for use in reducing adiposity in adipose tissue in a subject.

21. Use of a PSMD9 inhibitor in the manufacture of a medicament for use in reducing adipose tissue weight gain or promoting adipose tissue weight loss.

22. The method of claim 16, wherein the adipose tissue is visceral adipose tissue.
